SEQUENCE LISTING (48 sequences)

SEQ ID NO: 1 - protein, 128 aa, Artificial Sequence
  mAb13 HCVD
EVQLVESGGGLVQPGGSLRLSCAASGFTFSSYWMHWVRQAPGKGLVWVSRINSDGSTTRYADSVKGRFTISRDNAKNTLYLQMNSLRAEDTAVYYCARDDRGITMVRGIILFGFLDLWGRGTLVTVSS

SEQ ID NO: 2 - protein, 8 aa, Artificial Sequence
  mAb13 HCDR1
GFTFSSYW

SEQ ID NO: 3 - protein, 8 aa, Artificial Sequence
  mAb13 HCDR2
INSDGSTT

SEQ ID NO: 4 - protein, 21 aa, Artificial Sequence
  mAb13 HCDR3
ARDDRGITMVRGIILFGFLDL

SEQ ID NO: 5 - protein, 107 aa, Artificial Sequence
  mAb13 LCVD
EIVLTQSPAILSLCPGERATLSCRASQNVSSYLAWYQQKPGQAPRLLISDASNRATGIPARFSGSGSGTDFTLTINNLEPEDFAVYYCQQRSNWPQTFGQGTKVEIK

SEQ ID NO: 6 - protein, 6 aa, Artificial Sequence
  mAb13 LCDR1
QNVSSY

SEQ ID NO: 7 - protein, 9 aa, Artificial Sequence
  mAb13 LCDR3
QQRSNWPQT

SEQ ID NO: 8 - protein, 129 aa, Artificial Sequence
  mAb48 HCVD
QVQLVESGGGVVQPGRSLRLSCAASGFTFSNYGMHWVRQAPGKGLEWVAVIWYDGNNKYYTDSVKGRFTISRDNSKNTLYLQMNSLRAEDTAVYYCARDSKFTVVVARRRYYYYDMDVWGQGTTVTVSS

SEQ ID NO: 9 - protein, 8 aa, Artificial Sequence
  mAb48 HCDR1
GFTFSNYG

SEQ ID NO: 10 - protein, 8 aa, Artificial Sequence
  mAb48 HCDR2
IWYDGNNK

SEQ ID NO: 11 - protein, 22 aa, Artificial Sequence
  mAb48 HCDR3
ARDSKFTVVVARRRYYYYDMDV

SEQ ID NO: 12 - protein, 108 aa, Artificial Sequence
  mAb48 LCVD
EIVMTQSPATLSVSPGERATLSCRASQTISSNLAWYQQKPGQAPRLLIYGASTRATGIPARFSGSGSGTEFTLTISSLESEDFAVYYCQHYNRLPPWTFGQGTRVEIK

SEQ ID NO: 13 - protein, 6 aa, Artificial Sequence
  mAb48 LCDR1
QTISSN

SEQ ID NO: 14 - protein, 10 aa, Artificial Sequence
  mAb48 LCDR3
QHYNRLPPWT

SEQ ID NO: 15 - protein, 120 aa, Artificial Sequence
  mAb49 HCVD
QVQLVQSGAEVKKPGSSVKVSCKASGGTFSSYTISWVQQAPGQGLEWMVGIIPIFGTANYAQKFQSRVTITADESTSTAYMELSSLRSEDTAVYYCARSKVNFSGWYYYWGQGTLVTVSS

SEQ ID NO: 16 - protein, 8 aa, Artificial Sequence
  mAb49 HCDR1
GGTFSSYT

SEQ ID NO: 17 - protein, 8 aa, Artificial Sequence
  mAb49 HCDR2
IIPIFGTA

SEQ ID NO: 18 - protein, 13 aa, Artificial Sequence
  mAb49 HCDR3
ARSKVNFSGWYYY

SEQ ID NO: 19 - protein, 107 aa, Artificial Sequence
  mAb49 LCVD
EIVLTQSPATLSLSPGERATLSCRASQSVSSYLAWYQQKAGQAPRLLIYDASNRATGIPARFSGSGSGTDFTLTISRLEPEDFAVYYCQQRSTWPWTFGQGTKVEIK

SEQ ID NO: 20 - protein, 6 aa, Artificial Sequence
  mAb49 LCDR1
QSVSSY

SEQ ID NO: 21 - protein, 9 aa, Artificial Sequence
  mAb49 LCDR3
QQRSTWPWT

SEQ ID NO: 22 - DNA, 384 bp, Artificial Sequence
  mAb13 HCVD
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtaccacaaggtacgcggactccgtgaagggccgattcaccatctccagagacaacgccaagaatacgctgtatctgcaaatgaacagtctgagagccgaggacacggctgtgtattactgtgcaagagatgatagggggattactatggttcggggaattattctattcgggttcctcgatctctggggccgtggcaccctggtcactgtctcctca

SEQ ID NO: 23 - DNA, 24 bp, Artificial Sequence
  mAb13 HCDR1
ggattcaccttcagtagctactgg

SEQ ID NO: 24 - DNA, 24 bp, Artificial Sequence
  mAb13 HCDR2
attaatagtgatgggagtaccaca

SEQ ID NO: 25 - DNA, 63 bp, Artificial Sequence
  mAb13 HCDR3
gcaagagatgatagggggattactatggttcggggaattattctattcgggttcctcgatctc

SEQ ID NO: 26 - DNA, 322 bp, Artificial Sequence
  mAb13 LCVD
gaaattgtgttgacacagtctccagccatcctgtctttgtgtccaggggaaagagccaccctctcctgcagggccagtcagaatgttagcagctacttagcctggtaccaacagaaacctggccaggctcccaggctcctcatctctgatgcatccaacagggccactggcatcccagccaggttcagtggcagtgggtctgggacagacttcactctcaccatcaacaacctggagcctgaagattttgcagtttattactgtcagcagcgtagcaactggcctcagacgttcggccaagggaccaaggtggagatcaaac

SEQ ID NO: 27 - DNA, 18 bp, Artificial Sequence
  mAb13 LCDR1
cagaatgttagcagctac

SEQ ID NO: 28 - DNA, 27 bp, Artificial Sequence
  mAb13 LCDR3
cagcagcgtagcaactggcctcagacg

SEQ ID NO: 29 - DNA, 387 bp, Artificial Sequence
  mAb48 HCVD
caggtgcagctggtggagtctgggggaggcgtggtccagcctgggaggtccctgagactctcctgtgcagcgtctggattcaccttcagtaactatggtatgcactgggtccgccaggctccaggcaaggggctggagtgggtggcagttatatggtatgatggaaataataaatactatacagactccgtgaagggccgattcaccatctccagagacaattccaagaacaccctatatctgcaaatgaacagcctgagagccgaggacacggctgtgtattactgtgcgagagattccaaatttacagtagtggttgcacggcgacggtactactactacgatatggacgtctggggccaagggaccacggtcaccgtctcctca

SEQ ID NO: 30 - DNA, 24 bp, Artificial Sequence
  mAb48 HCDR1
ggattcaccttcagtaactatggt

SEQ ID NO: 31 - DNA, 24 bp, Artificial Sequence
  mAb48 HCDR2
atatggtatgatggaaataataaa

SEQ ID NO: 32 - DNA, 66 bp, Artificial Sequence
  mAb48 HCDR3
gcgagagattccaaatttacagtagtggttgcacggcgacggtactactactacgatatggacgtc

SEQ ID NO: 33 - DNA, 325 bp, Artificial Sequence
  mAb48 LCVD
gaaatagtgatgacgcagtctccagccaccctgtctgtgtctccaggggaaagagccaccctctcctgcagggccagtcagactattagcagcaacttagcctggtaccagcagaaacctggccaggctcccaggctcctcatctatggtgcatccaccagggccactggtatcccagctaggttcagtggcagtgggtctgggacagagttcactctcaccatcagcagcctggagtctgaagattttgcagtttattactgtcaacactataataggctgcctccgtggacgttcggccaagggaccagggtggaaatcaaac

SEQ ID NO: 34 - DNA, 18 bp, Artificial Sequence
  mAb48 LCDR1
cagactattagcagcaac

SEQ ID NO: 35 - DNA, 30 bp, Artificial Sequence
  mAb48 LCDR3
caacactataataggctgcctccgtggacg

SEQ ID NO: 36 - DNA, 360 bp, Artificial Sequence
  mAb49 HCVD
caggtgcagctggtgcagtctggggctgaggtgaagaagcctgggtcctcggtgaaggtctcctgcaaggcttctggaggcaccttcagcagctatactatcagctgggtgcaacaggcccctggacaagggcttgagtggatggtagggatcatccctatctttggtacagcaaactacgcacagaagttccagagcagagtcacgattaccgcggacgaatccacgagcacagcctacatggagctgagcagcctgagatctgaggacacggccgtgtattactgtgcgagatcgaaagttaatttcagtggctggtactactactggggccagggaaccctggtcaccgtctcctca

SEQ ID NO: 37 - DNA, 24 bp, Artificial Sequence
  mAb49 HCDR1
ggaggcaccttcagcagctatact

SEQ ID NO: 38 - DNA, 24 bp, Artificial Sequence
  mAb49 HCDR2
atcatccctatctttggtacagca

SEQ ID NO: 39 - DNA, 39 bp, Artificial Sequence
  mAb49 HCDR3
gcgagatcgaaagttaatttcagtggctggtactactac

SEQ ID NO: 40 - DNA, 322 bp, Artificial Sequence
  mAb49 LCVD
gaaattgtgttgacacagtctccagccaccctgtctttgtctccaggggaaagagccaccctctcctgcagggccagtcagagtgttagcagctacttagcctggtaccaacagaaagctggccaggctcccaggctcctcatctatgatgcatccaacagggccactggcatcccagccaggttcagtggcagtgggtctgggacagacttcactctcaccatcagcaggctagagcctgaagattttgcagtttattactgtcagcagcgtagcacctggccgtggacgttcggccaagggaccaaggtggaaatcaaac

SEQ ID NO: 41 - DNA, 18 bp, Artificial Sequence
  mAb49 LCDR1
cagagtgttagcagctac

SEQ ID NO: 42 - DNA, 27 bp, Artificial Sequence
  mAb49 LCDR3
cagcagcgtagcacctggccgtggacg

SEQ ID NO: 43 - protein, 26 aa, Artificial sequence
  virus-derived peptides
VEGFNCYFPLQSYGFQPTNGVGYQPY

SEQ ID NO: 44 - protein, 30 aa, Artificial sequence
  virus-derived peptides
KVGGNYNYLYRLFRKSNLKPFERDISTEIY

SEQ ID NO: 45 - protein, 31 aa, Homo sapiens
  C-Peptide
EAEDLQVGQVELGGGPGAGSLQPLALEGSLQ

SEQ ID NO: 46 - protein, 29 aa, Mus musculus
  C-Peptide
EVEDPQVEQLELGGSPGDLQTLALEVARQ

SEQ ID NO: 47 - protein, 21 aa, Homo sapiens
  Insulin-A chain
GIVEQCCTSICSLYQLENYCN

SEQ ID NO: 48 - protein, 21 aa, Mus musculus
  Insulin-A chain
GIVDQCCTSICSLYQLENYCN

(19) United States
(12) Patent Application Publication
(10) Pub. No.: US 2025/0270311 A1
(43) Pub. Date: Aug. 28, 2025

(54) METHOD AND MEANS FOR MODULATING B-CELL MEDIATED IMMUNE RESPONSES

(71) Applicants: VACCINVENT GMBH, Freiburg im Breisgau (DE); UNIVERSITÄT ULM, Ulm (DE)

(72) Inventors: Hassan JUMAA-WEINACHT, Ulm (DE); Timm AMENDT, London (GB); Marc YOUNG, Weilheim Teck (DE)

(21) Appl. No.: 18/274,624

(22) PCT Filed: Jan. 28, 2022
(86) PCT No.: PCT/EP2022/052146

§ 371 Date: Jul. 27, 2023

(30) Foreign Application Priority Data
Jan. 28, 2021 (WO) PCT/EP2021/052000
Aug. 5, 2021 (EP) 21189995.0

(51) Int. Cl. C07K 16/26; A61P 3/10

(52) U.S. Cl. CPC C07K 16/26; A61P 3/10; C07K 2317/24

(57) ABSTRACT

The invention pertains to methods and means for the targeted modulation of B-cell mediated immune responses by bringing into contact a B-cell with a specific ratio of soluble single monovalent antigens and complexed multivalent antigens. The targeted modulation of B-cell immunity can be used in mammals for the diagnosis and therapy of various conditions associated with antibody-mediated immunity. Such conditions include proliferative disorders such as cancer, autoimmune disorders, pathogenic infections, inflammatory diseases, allergies and food intolerances. The invention is predicated on the observation that complexed multivalent antigenic structures induce a strong IgG type antibody B-cell response while surprisingly monovalent antigenic structures harbour the ability to suppress such IgG responses, or even induce in the case of autoantigens protective IgM responses, in particular protective oligomeric anti-insulin antibodies. The invention in this regard offers methods, compositions, therapeutics, diagnostics and food additives.

### RELATED APPLICATIONS

The instant application is a 35 U.S.C. § 371 filing of International Patent Application No. PCT/EP2022/052146, filed Jan. 28, 2022, which claims priority to European Patent Application No. 21189995.0, filed Aug. 5, 2021, and International Patent Application No. PCT/EP2021/052000, filed Jan. 28, 2021, the entire contents of which are incorporated herein by reference for all purposes.

### SEQUENCE LISTING

The instant application contains a Sequence Listing which has been submitted electronically in TXT format and is hereby incorporated by reference in its entirety. Said ASCII file, created on Jan. 30, 2024, is named 745077-VOS9-014US_ST25.txt and is 17,868 bytes in size.

### FIELD OF THE INVENTION

The invention pertains to methods and means for the targeted modulation of B-cell mediated immune responses by bringing into contact a B-cell with a specific ratio of soluble single monovalent antigens and complexed multivalent antigens. The targeted modulation of B-cell immunity can be used in mammals for the diagnosis and therapy of various conditions associated with antibody-mediated immunity. Such conditions include proliferative disorders such as cancer, autoimmune disorders, pathogenic infections, inflammatory diseases, allergies and food intolerances. The invention is predicated on the observation that complexed multivalent antigenic structures induce a strong IgG type antibody B-cell response while surprisingly monovalent antigenic structures harbour the ability to suppress such IgG responses, or even induce in the case of autoantigens protective IgM responses, in particular protective oligomeric anti-insulin antibodies. The invention in this regard offers methods, compositions, therapeutics, diagnostics and food additives.

### DESCRIPTION

Self-tolerance is crucial for maintaining physiological integrity by avoiding autoimmune reactions. Currently, absolute central and peripheral tolerance are believed to control the B cell receptor (BCR) repertoire during B cell development thereby preventing positive selection of self-reactive B cells [1,2,4]. It is assumed that central tolerance forces deletion of autoreactive B cells during early B cell development in the bone marrow [2,5-7]. Furthermore, autoreactive B cells escaping clonal deletion are subjected to receptor editing resulting in non-autoreactive BCR specificities [8-10]. Self-reactive B cells that circumvent central tolerance and migrate to the periphery are counteracted by clonal anergy (peripheral tolerance) leading to unresponsiveness mainly by downmodulation of IgM BCR expression [1,11-13]. However, the finding that the vast majority of serum IgM is autoreactive seems to contrast the concept of general elimination of autoreactivity [14]. In fact, the so-called natural polyreactive IgM plays important roles in homeostasis [15] arguing against the absolute elimination of autoreactive antibodies.

Interestingly, it has been shown that disease-specific autoreactive B cells are present within the pre-immune repertoire and that germinal centers (GC) specific for insulin, a common autoantigen, can be formed in wildtype mice contradicting the concept of central B cell tolerance [16,17].

In the past decades, B cell autoimmunity research focused largely on transgenic mouse models [1,2,5,18,19]. The usefulness of these models for studying autoimmunity has been heavily debated for several reasons [20]. Replacement of the germline configuration by a high-affinity mutated autoreactive BCR not only leads to an atypical situation during B cell development, it also generates a monospecific repertoire [1,5,19]. Moreover, the characteristics of these antigens with regard to their availability, valency and form (soluble vs. membrane-bound) have not been adequately addressed [5, 18]. Furthermore, the antigens themselves do not have any relevance to known autoimmune diseases [21, 22].

Epidemiological studies show that up to 5% of the population in industrialized countries suffers from autoimmune diseases such as rheumatoid arthritis (RA), systemic lupus erythematosus (SLE), or type-1-diabetes (T1D) [21]. Notably, autoantibodies are present in the vast majority of autoimmune diseases and often are the driving force of pathogenesis [22]. Furthermore, anti-insulin antibodies play a critical role for insulin activity, development of diabetes and insulin treatment [57-59].

Hence, there is a continued need to develop approaches for a controllable modulation of immune responses in order to detect or treat or avoid conditions that are induced or characterized by the presence or activity of immune responses in a subject, in particular immune responses against insulin.

### BRIEF DESCRIPTION OF THE INVENTION

Generally, and by way of brief description, the main embodiments of the present invention can be described as follows:
1. An oligomeric anti-insulin antibody, wherein the antibody
   (i) has an affinity to insulin and/or proinsulin of Kd<5×10⁻⁷, preferably as measured by surface plasmon resonance; and/or
   (ii) is monospecific for insulin and/or proinsulin.
2. The oligomeric anti-insulin antibody of embodiment 1, wherein the oligomeric anti-insulin antibody is an anti-insulin antibody of the IgM isotype.
3. The oligomeric anti-insulin antibody of embodiment 1 or 2, wherein the oligomeric anti-insulin antibody is chimeric, humanized or human.
4. The oligomeric anti-insulin antibody of embodiments 1 to 3, wherein the immunoglobulin comprises
   a) a variable heavy (VH) chain comprising CDR1 as defined in SEQ ID NO: 2, CDR2 as defined in SEQ ID NO: 3 and CDR3 as defined in SEQ ID NO: 4 and a variable light (VL) chain comprising CDR1 as defined in SEQ ID NO: 6, CDR2 as defined by the sequence DAS and CDR3 as defined in SEQ ID NO: 7;
   b) a variable heavy (VH) chain comprising CDR1 as defined in SEQ ID NO: 9, CDR2 as defined in SEQ ID NO: 10 and CDR3 as defined in SEQ ID NO: 11 and a variable light (VL) chain comprising CDR1 as defined in SEQ ID NO: 13, CDR2 as defined by the sequence GAS and CDR3 as defined in SEQ ID NO: 14; or
   c) a variable heavy (VH) chain comprising CDR1 as defined in SEQ ID NO: 16, CDR2 as defined in SEQ ID NO: 17 and CDR3 as defined in SEQ ID NO: 18 and a variable light (VL) chain comprising CDR1 as defined in SEQ ID NO: 20, CDR2 as defined by the sequence DAS and CDR3 as defined in SEQ ID NO: 21.
5. The oligomeric anti-insulin antibody of embodiment 4, wherein the oligomeric anti-insulin antibody comprises
   a) comprises a variable heavy (VH) chain sequence comprising the amino acid sequence of SEQ ID NO: 1 or a sequence having at least 90%, preferably at least 95% sequence identity to SEQ ID NO: 1 and a variable light (VL) chain sequence comprising the amino acid sequence of SEQ ID NO: 4 or a sequence having at least 90%, preferably at least 95% sequence identity to SEQ ID NO: 4;
   b) comprises a variable heavy (VH) chain sequence comprising the amino acid sequence of SEQ ID NO: 8 or a sequence having at least 90%, preferably at least 95% sequence identity to SEQ ID NO: 8 and a variable light (VL) chain sequence comprising the amino acid sequence of SEQ ID NO: 12 or a sequence having at least 90%, preferably at least 95% sequence identity to SEQ ID NO: 12; or
   c) comprises a variable heavy (VH) chain sequence comprising the amino acid sequence of SEQ ID NO: 15 or a sequence having at least 90%, preferably at least 95% sequence identity to SEQ ID NO: 15 and a variable light (VL) chain sequence comprising the amino acid sequence of SEQ ID NO: 19 or a sequence having at least 90%, preferably at least 95% sequence identity to SEQ ID NO: 19.
6. A polynucleotide that encodes an oligomeric anti-insulin antibody of any one of embodiments 1 to 5.
7. A host cell comprising the polynucleotide of embodiment 6.
8. A method for producing an oligomeric anti-insulin antibody comprising culturing the host cell of embodiment 7.
9. A pharmaceutical composition comprising the oligomeric anti-insulin antibody of any one of embodiments 1 to 5, the polynucleotide of embodiment 6, the host cell of embodiment 7, and a pharmaceutically acceptable carrier.
10. The pharmaceutical composition of embodiment 9 comprising a further therapeutic agent.
11. The oligomeric anti-insulin antibody of any one of embodiments 1 to 5, the polynucleotide of embodiment 6, the host cell of embodiment 7, or the pharmaceutical composition of embodiments 9 to 10 for use in treatment.
12. The oligomeric anti-insulin antibody of any one of embodiments 1 to 5, the polynucleotide of embodiment 6, the host cell of embodiment 7, or the pharmaceutical composition of embodiments 9 to 10 for use in the treatment of an insulin-associated disease or disorder.
13. A method of diagnosing and/or predicting an insulin-associated disease or disorder, the method comprising the steps of:
   (i) determining the affinity of the binding of anti-insulin IgM antibodies to proinsulin and/or insulin from a sample,
      wherein the sample has been obtained from a subject, wherein the subject is diagnosed with an insulin-associated disease or disorder or is at risk thereof;
   (ii) comparing the level(s) determined in step (i) to a reference value; and
   (iii) diagnosing and/or predicting an insulin-associated disease or disorder in said subject based on the comparison made in step (ii), preferably wherein a lower affinity of the binding of anti-insulin IgM antibodies to proinsulin and/or insulin indicates a higher risk for an insulin-associated disease or disorder.
14. A method for determining whether a subject is susceptible to a treatment of insulin-associated disease or disorder, the method comprising the steps of:
   (i) determining the affinity of the binding of anti-insulin IgM antibodies to proinsulin and/or insulin from a sample,
      wherein the sample has been obtained from a subject, wherein the subject is diagnosed with an insulin-associated disease or disorder or is at risk thereof;
   (ii) comparing the level(s) determined in step (i) to a reference value; and
   (iii) determining whether said subject is susceptible to the treatment of insulin-associated disease or disorder, preferably wherein a lower affinity of the binding of anti-insulin IgM antibodies to proinsulin and/or insulin indicates a higher susceptibility to the treatment of insulin-associated disease or disorder.
15. The oligomeric anti-insulin antibody for use of embodiments 12, the polynucleotide for use of embodiment 12 or the host cell for use of embodiment 12, or the pharmaceutical composition for use of embodiment 12, the method of embodiment 13 or 14, wherein the insulin-associated disease or disorder is selected from the group of pancreatic damage, type 1 diabetes, type 2 diabetes, exogenous insulin antibody syndrome, gestational diabetes, and dysglycemia.
16. The oligomeric anti-insulin antibody for use of embodiments 15, the polynucleotide for use of embodiment 15 or the host cell for use of embodiment 15, the pharmaceutical composition for use of embodiment 15 or the method of embodiment 15, wherein the dysglycemia is dysglycemia in a patient with an insulin-associated disease or disorder is selected from the group of pancreatic damage, type 1 diabetes, type 2 diabetes, exogenous insulin antibody syndrome and gestational diabetes.
17. A method for producing an oligomeric anti-insulin and/or anti-proinsulin antibody, preferably of the IgM isotype, comprising immunizing a mammal with a mixture of at least one monovalent insulin particle and at least one polyvalent insulin particle.
18. A method for treatment and/or prevention of an insulin-associated disease or disorder, the method comprising a step of administering a therapeutically effective amount, of the oligomeric anti-insulin antibody of any one of embodiments 1 to 5, the polynucleotide of embodiment 6, the host cell of embodiment 7, or the pharmaceutical composition of embodiments 9 to 10.

### DETAILED DESCRIPTION OF THE INVENTION

In the following, the elements of the invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

Accordingly, the invention relates to an oligomeric anti-insulin antibody, wherein the antibody (i) has an affinity to insulin and/or proinsulin of Kd<5×10⁻⁷.

In some embodiments, the invention relates to an oligomeric anti-insulin antibody, wherein the antibody (i) has an affinity to insulin and/or proinsulin of Kd<5×10⁻⁷, preferably as measured by surface plasmon resonance; and/or (ii) is monospecific for insulin and/or proinsulin.

In some embodiments, the invention relates to an oligomeric anti-insulin antibody, wherein the antibody (i) has an affinity to insulin and/or proinsulin of Kd<5×10⁻⁷; and/or (ii) is monospecific for insulin and/or proinsulin.

The term “monospecific” in context of antibodies as used herein denotes an antibody that has one or more binding sites each of which bind to the same epitope of the same antigen. More importantly, the term “monospecific” in context of the present invention pertains to such an antibody which has a high affinity to one antigen such as insulin and which does not bind specifically to any other antigen. In this embodiment a monospecific antibody binds to the antigen associated with the autoimmune disorder such as insulin with a K_{D} of less than 10⁻⁷ nM, preferably of less than 10⁻⁸ nM, more preferably of less than 10⁻⁹ nM and most preferably of about 10⁻¹⁰ nM. Hence, such monoclonal IgM does not bind to an unrelated antigen, which is an antigen other than the antigen associated with the autoimmune disorder, and preferably the treatment if the invention therefore does not comprise the use of a polyspecific antibody specific for an unrelated antigen which is an antigen other than the antigen associated with the autoimmune disorder. In some embodiments, monospecificity of an antibody is defined in that it does not recognize dsDNA in ELISA and shows no binding in Hep-2 slides (see e.g. Example 4, FIG. 16C, 16D and Material and Methods).

The term “K_{D}”, as used herein, is intended to refer to the dissociation constant, which is obtained from the ratio of Kd to Ka (i. e., Kd/Ka) and is expressed as a molar concentration (M). KD values for antibodies can be determined using methods well established in the art such as plasmon resonance (BIAcore®), Bio-Layer Interferometry (BLI), ELISA and KINEXA. A preferred method for determining the KD of an antibody is by using surface plasmon resonance, preferably using a biosensor system such as a BIAcore® system or by ELISA. “Ka” (or “K-assoc”), as used herein, refers broadly to the association rate of a particular antibody-antigen interaction, whereas the term “Kd” (or “K-diss”), as used herein, refers to the dissociation rate of a particular antibody-antigen interaction. Another preferred method is the use of BLI. The term “bio-layer interferometry” or “BLI” refers to an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. In some embodiments, the Kd is measured by surface plasmon resonance.

The insulin described herein can of any source. In some embodiments the insulin described herein is a mammalian insulin, a partially or fully synthetic insulin, preferably human insulin. In some embodiments, the insulin described herein is an insulin variant or an insulin analogue such as an insulin analogue selected from the group of aspart, lispro, glulisine, glargine, detemir, degludec.

The anti-insulin antibody described herein can also be a anti-proinsulin antibody or an anti-proinsulin and anti-insulin antibody.

The term “proinsulin”, as used herein, refers to an insulin polypeptide which includes the connecting peptide or “C-peptide” linking the B and A insulin polypeptide chains.

The inventors demonstrate that insulin activity is regulated by different anti-insulin antibodies in healthy and diabetic subjects (see e.g. Example 6, 7). Herein provided are the means and methods to use and/or influence this regulatory system. In particular the inventors demonstrate that an oligomeric anti-insulin antibody binding with a monospecific and/or high affinity binding to insulin has a protective effect on insulin function. Without being bound to theory the oligomeric anti-insulin antibody described herein protects insulin from degradation upon binding of less selective and/or specific antibodies (Example 7).

Accordingly, the invention is at least in part based on the protective/regulative effect of the oligomeric anti-insulin antibody on insulin activity.

In some embodiments, the invention pertains to a method of eliciting and/or modulating a cell-mediated target antigen-specific immune response in a subject, the method comprising contacting one or more immune-cells (such as B-cells) of the subject with a combination comprising:
(i) a monovalent antigen particle which is composed of an antigenic portion comprising not more than one of an antigenic structure capable of inducing an antibody mediated immune response against the disease-associated antigen, and
(ii) a polyvalent antigen particle which is composed of an antigenic portion comprising more than one of an antigenic structure capable of inducing an antibody mediated immune response against the disease-associated antigen and wherein the more than one of an antigenic structure are covalently or non-covalently cross-linked.

In some embodiments, which is an alternative to the first aspect, the invention pertains a combination for use in eliciting and/or modulating a cell-mediated target antigen-specific immune response in a subject, the combination comprising
(i) a monovalent antigen particle which is composed of an antigenic portion comprising not more than one of an antigenic structure capable of inducing an antibody mediated immune response against the disease-associated antigen, and
(ii) a polyvalent antigen particle which is composed of an antigenic portion comprising more than one of an antigenic structure capable of inducing an antibody mediated immune response against the disease-associated antigen and wherein the more than one of an antigenic structure are covalently or non-covalently cross-linked;
   wherein the combination is used by contacting one or more immune-cells of the subject with the combination.

In a further alternative of the first and the second aspect, the combination is for use in the treatment or prevention (vaccination) of a disease in a subject or patient comprises the administration of the combination or of at least (i) or (ii) of the combination to the subject or patient in a therapeutically or preventively effective amount. A therapeutically effective amount in context of the present invention is an amount that induces or suppresses a certain B-cell mediated immune response such as an IgG- or IgM-type (or an IgA) immune response.

The present invention is predicated upon the surprising finding that antigens may induce different immune responses depending on whether they are presented to immune cells as soluble antigens or as complexed multivalent antigens. The latter in particular lead to strong and memory IgG antibody responses, whereas the former may repress such IgG response and induce a protective IgM (or an IgA) antibody response. Hence, the invention suggests to modulate the ratio soluble to complexed immune responses in order control the focus of B-cell immunity. The approach may be used in novel controlled vaccination treatments or for tackling autoimmune diseases such as diabetes.

The method described herein is in some embodiments a non-therapeutic and non-surgical method. In this embodiment, the method of the invention is not for treating a subject but for inducing an immune response for, for example, the production and isolation of novel antibodies which are isolated in a subsequent step. In this embodiment, the subject is a generally healthy subject not suffering from any disease which is treated by performing the method. In this embodiment the subject is preferably a non-human vertebrate.

A “cell-mediated target antigen-specific immune response” in context of the present invention shall refer to an immune response involving one or more B lymphocytes (B-cell), and preferably, a B-cell-mediated immune response. The term “B lymphocyte” or “B cell”, as used herein, refers to a lymphocyte that plays a role in humoral immunity of the adaptive immune system, and which is characterised by the presence of the B cell receptor (BCR) on the cell surface. B cell types include plasma cells, memory B cells, B-1 cells, B-2 cells, marginal-zone B cells, follicular B cells, and regulatory B cells (B_{reg}).

The term “valent” as used within the current application denotes the presence of a specified number of binding sites in an antibody or antigen, respectively, molecule. As such a binding site of an antibody is a paratope, whereas a binding site in the antigen is generally referred to as epitope. A natural antibody for example or a full length antibody according to the invention has two binding sites and is bivalent. Antigen proteins are monovalent (when present as monomers), however, if such antigen proteins are provided as multimers they may comprise more than one identical epitope and therefore are polyvalent, which may be bivalent, trivalent, tetravalent etc. As such, the terms “trivalent”, denote the presence of three binding sites in an antibody molecule. As such, the terms “tetravalent”, denote the presence of four binding sites in an antibody molecule.

The term “monovalent antigen particle” shall in context of the herein disclosed invention refer to a molecule or molecule-complex, such as a protein, or protein complexes, which are antigenic, and therefore capable of stimulating an immune response in a vertebrate. Typically, a monovalent antigen particle is composed of an antigenic portion comprising not more than one of an antigenic structure capable of inducing an antibody mediated immune response against such antigenic structure. As used herein, the term “antigenic structure” refers to fragment of an antigenic protein that retains the capacity of stimulating an antibody mediated immune response. Such an antigenic structure is understood to provide the antigenic determinant or “epitope” which refers to the region of a molecule that specifically reacts with an antibody, more specifically that reacts with a paratope of an antibody. In preferred embodiments of the invention a monovalent antigen particle of the invention comprises not more than one copy of one specific epitope of the antigenic structure. Hence, preferably only one antibody molecule of a certain antibody species having a specific paratope may bind to a monovalent antigen particle according to the invention.

The term “polyvalent antigen particle” shall in context of the herein disclosed invention refer to a molecule or molecule-complex, such as a protein, or protein complexes, which are antigenic, and therefore capable of stimulating an immune response in a vertebrate. In the invention, unlike monovalent antigenic particles, a polyvalent antigenic particle is composed of an antigenic portion comprising more than one of an antigenic structure capable of inducing an antibody mediated immune response. In preferred embodiments of the invention a polyvalent antigen particle of the invention comprises more than one copy of one specific epitope of the antigenic structure. Hence, preferably more than one antibody molecule of a certain antibody species having a specific paratope may bind to a monovalent antigen particle according to the invention. Such polyvalent antigen particle may have a structure that the more than one of an antigenic structure are covalently or non-covalently cross-linked with each other. A polyvalent antigen particle therefore, in preferred embodiments comprises complex comprising at least two, at least three or at least four identical epitopes, which allow for a binding of two antibodies to the polyvalent antigen particle at the same time. Preferably, the more than one of an antigenic structure comprised in the antigenic portion of the polyvalent antigen particle comprises multiple identical antigenic structures.

A polyvalent-antigen particle of the invention preferably comprises the at least two copies of the antigenic structure in spatial proximity to each other, preferably within a nanometer range selected from the ranges 1 nm to 10 μm. more preferably 1 nm to 5 μm, 1 nm to 1000 nm, 1 nm to 500 nm, 1 nm to 100 nm, 1 nm to 50 nm and 1 nm to 10 nm.

In context of the invention the monovalent antigen particle of the invention is often referred to as “soluble” particle or antigen whereas the polyvalent antigen particle is referred to as “complexed” particle or antigen.

In another embodiment of the invention, the monovalent-antigen particle further comprises a carrier portion which is coupled to the antigenic portion, optionally via a linker, and wherein the carrier, and optionally the linker, does not comprise another copy of the antigenic structure, and wherein the carrier portion, and optionally the linker, is not capable of eliciting a cell-mediated immune response against the target antigen. In another alternative or additional embodiment of the invention, the polyvalent-antigen particle further comprises a carrier portion which is coupled to the antigenic portion, optionally via a linker. A “linker” in context of the present invention may comprise any molecule, or molecules, proteins or peptides which may be used to covalently or non-covalently connect two portions of the compounds of the invention with each other.

The term “carrier portion” in context of the herein disclosed invention preferably relates to a substance or structure that presents or comprises the antigenic structures of the particles of the invention. A carrier portion is preferably a substance or structure selected from immunogenic or non-immunogenic polypeptides, immune CpG islands, limpet hemocyanin (KLH), tetanus toxoid (TT), cholera toxin subunit B (CTB), bacteria or bacterial ghosts, liposome, chitosome, virosomes, microspheres, dendritic cells, particles, microparticles, nanoparticles, or beads.

Preferably, neither the carrier portion, and optionally also not the linker, is (are) capable of eliciting a cell-mediated immune response against the target antigen, such as the antigen associated with an autoimmune disorder.

A “linker” in context of the invention is preferably peptide linker which may have any size and length suitable for a given application in context of the invention. Linkers may have a length or 1-100 amino acids, preferably of 2 to 50 amino acids. A linker could be a typical 4GS linker in 2, 3, 4, 5, 6 or more repeats.

In preferred embodiments of the invention the contacting one or more immune-cells of the subject or patient with a combination comprising a monovalent-antigen particle and a polyvalent-antigen particle involves (i) administration of the monovalent-antigen particle to the subject, (ii) administration of the polyvalent-antigen particle to the subject, or (iii) administration of the monovalent-antigen particle and the polyvalent-antigen particle to the subject, wherein in (i), (ii) and (iii), the immune cells of the subject are as a result of the administration in contact with the combination the monovalent-antigen particle and the polyvalent-antigen particle. In general, the term “contacting” shall be understood to present such antigen particles to the immune system of the subject in order to induce preferably a B-cell mediated immune response. Preferably, in (i) the subject is characterized by the presence of the polyvalent-antigen particle before administration of the monovalent-antigen particle, and in (ii) the subject is characterized by the presence of the monovalent-antigen particle before administration of the polyvalent-antigen particle.

In context of the present invention, it was found that a specific ratio of monovalent and polyvalent antigen can modulate antibody immune responses mediated by B-cells. Hence, it is a preferred embodiment of the invention the combination comprising the monovalent-antigen particle and the polyvalent-antigen particle comprises a specific antigen-ratio, which is preferably a ratio of monovalent-antigen particle to polyvalent-antigen particle. In particular of such preferred embodiments modulating the cell-mediated target antigen-specific immune response in the subject constitutes a control of an IgG-type (or IgM) target antigen-specific B-cell response in the subject by contacting one or more of the B-cells of the subject with a combination comprising a specific antigen-ratio which is greater than 1, preferably greater than 10¹, 10², 10³, 10⁴ or more. In other embodiments of the invention the contacting one or more of the B-cells of the subject with the combination involves administering to the subject an amount of monovalent-antigen particle which is effective to generate in the subject a specific antigen-ratio which is greater than 1, preferably greater than 10¹, 10², 10³, 10⁴ or more.

In further particular embodiments of the invention, the method is preferred wherein the contacting one or more of the B-cells of the subject with the amount of monovalent-antigen particle is administered either with or without a direct combination of administering polyvalent-antigen particle to the subject.

In context of the present invention modulating the cell-mediated target antigen-specific immune response in the subject constitutes preferably an increasing of an IgG-type target antigen-specific B-cell response in the subject by contacting one or more of the B-cells of the subject with a combination comprising a specific antigen-ratio which is less than 1, preferably less than 10⁻¹, 10⁻², 10⁻³, 10⁻⁴ or less. Preferably wherein the contacting one or more of the B-cells of the subject with the combination involves administering to the subject an amount of polyvalent-antigen particle which is effective to generate in the subject a specific antigen-ratio which is less than 1, preferably less than 10⁻¹, 10⁻², 10⁻³, 10⁻⁴ or less.

It is preferred that the contacting one or more of the B-cells of the subject with the amount of polyvalent-antigen particle is administered either with or without a direct combination of administering monovalent-antigen particle to the subject.

The term “antigen” may refer to any, preferably disease associated, molecule or structure that comprises an antigenic structure. Preferably an antigen of the invention is an autoantigen, a cancer associated antigen, or a pathogen associated antigen. In one very specific exemplary embodiment of the invention the antigen is insulin and the associated disease is diabetes. Human insulin protein is produced as proinsulin comprising a c-peptide, insulin B chain and and the active insulin peptide. The amino acid sequence and further characteristics is well known to the skilled artisan and can be derived under accession no. P01308 in the UniProt database in the Version of Jan. 27, 2020 (https://www.uniprot.org/uniprot/P01308).

A pathogen associated antigen of the invention may be any antigen that is expressed in, on or by a pathogen, such as a pathogenic virus or microorganism, preferably wherein the pathogen is selected from a parasite, a monocellular eukaryote, a bacterium, a virus or virion.

The antigen of the invention is preferably an antigen which is associated with a disease or condition, preferably a disease or condition the subject suffers or is suspected to suffer from. Such disease, as mentioned, may be pathogen associated, autoimmune associated, might by associated with a treatment, for example when using an antigenic protein as therapeutic such as a therapeutic antibody, or cancer associated or the like. An antigen of the invention can be a natural or synthetic immunogenic substance, such as a complete, fragment or portion of an immunogenic substance, and wherein the immunogenic substance may be selected from a nucleic acid, a carbohydrate, a peptide, a hapten, or any combination thereof.

In context of the invention the disease or condition is selected from a disease or condition which is characterized in that an increased or reduced cell-mediated immune response is beneficial for a treatment. Hence, the invention offers the herein described modulation of the immune system according to the herein described methods as a treatment of diseases such as a disease or condition selected from an inflammatory disorder, an autoimmune disease, a proliferative disorder, or an infectious disease.

The term “B cell” (also known as a “B lymphocyte”) refers to immune cells which express a cell surface immunoglobulin molecule and which, upon activation, terminally differentiate into cells, which secrete antibody. Accordingly, this includes, for example, convention B cells, CD5 B cells (also known as B-1 cells and transitional CD5 B cells). “B cell” should also be understood to encompass reference to B cell mutants. “Mutants” include, but are not limited to, B cells which have been naturally or non-naturally modified, such as cells which are genetically modified. Reference to “B cells” should also be understood to extend to B cells which exhibit commitment to the B cell image. These cells may be at any differentiative stage of development and therefore may not necessarily express a surface immunoglobulin molecule. B cell commitment may be characterized by the onset of immunoglobulin gene re-arrangement or it may correspond to an earlier stage of commitment which is characterized by some other phenotypic or functional characteristic such as the cell surface expression of CD45R, MHCII, CD10, CD19 and CD38. Examples of B cells at various stages of differentiation include early B cell progenitors, early pro-B cells, late pro-B cells, pre-B cells, immature B cells, mature B cells, plasma cells, and memory (B) cells. In context of the present invention a B-cell can be seen as a non-maturated B-cell expressing mainly IgM type B-cell receptor, a maturated B-cell expressing mainly IgD type B-cell receptor or memory B-cell expressing IgG type B-cell receptor. The difference between the IgM type and IgD type B-cell receptor is the type of heavy chain sequence which either is of the μ or δ type.

In context of the invention the term “cell-mediated target antigen-specific immune response” preferably pertains to a cellular immune type response involving an immune cell such as a lymphocyte, preferably a B lymphocyte (B-cell mediated immune response), preferably which comprises and/or expresses one or more antibody, or variants thereof, and/or B cell receptors, and/or variants thereof, which are specific for the target antigen. Preferably a cell-mediated target antigen-specific immune response involves a B cell expressing a Immunoglobulin (Ig) M, IgD, IgA or IgG type antibody and/or B-cell receptor.

As used herein, the term “antibody” may be understood in the broadest sense as any immunoglobulin (Ig) that enables binding to its epitope. An antibody as such is a species of an ABP. Full length “antibodies” or “immunoglobulins” are generally heterotetrameric glycoproteins of about 150 kDa, composed of two identical light and two identical heavy chains. Each light chain is linked to a heavy chain by one covalent disulphide bond, while the number of disulphide linkages varies between the heavy chain of different immunoglobulin isotypes. Each heavy and light chain also has regularly spaced intrachain disulphide bridges. Each heavy chain has an amino terminal variable domain (VH) followed by three carboxy terminal constant domains (CH). Each light chain has a variable N-terminal domain (VL) and a single C-terminal constant domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to cells or factors, including various cells of the immune system (e.g., effector cells) and the first component (C1q) of the classical complement system. Other forms of antibodies include heavy-chain antibodies, being those which consist only of two heavy chains and lack the two light chains usually found in antibodies. Heavy-chain antibodies include the hcIgG (IgG-like) antibodies of camelids such as dromedaries, camels, llamas and alpacas, and the IgNAR antibodies of cartilaginous fishes (for example sharks). And yet other forms of antibodies include single-domain antibodies (sdAb, called Nanobody by Ablynx, the developer) being an antibody fragment consisting of a single monomeric variable antibody domain. Single-domain antibodies are typically produced from heavy-chain antibodies, but may also be derived from conventional antibodies.

Typical antibody Ig variants discussed in context of the invention comprise IgG, IgM, IgE, IgA, or IgD antibodies.

As used herein, the term “IgG” has its general meaning in the art and refers to an immunoglobulin that possesses heavy g-chains. Produced as part of the secondary immune response to an antigen, this class of immunoglobulin constitutes approximately 75% of total serum Ig. IgG is the only class of Ig that can cross the placenta in humans, and it is largely responsible for protection of the newborn during the first months of life. IgG is the major immunoglobulin in blood, lymph fluid, cerebrospinal fluid and peritoneal fluid and a key player in the humoral immune response. Serum IgG in healthy humans presents approximately 15% of total protein beside albumins, enzymes, other globulins and many more. There are four IgG subclasses described in human, mouse and rat (e.g. IgG1, IgG2, IgG3, and IgG4 in humans). The subclasses differ in the number of disulfide bonds and the length and flexibility of the hinge region. Except for their variable regions, all immunoglobulins within one class share about 90% homology, but only 60% among classes. IgG1 comprises 60 to 65% of the total main subclass IgG, and is predominantly responsible for the thymus-mediated immune response against proteins and polypeptide antigens. IgG1 binds to the Fc-receptor of phagocytic cells and can activate the complement cascade via binding to C1 complex. IgG1 immune response can already be measured in newborns and reaches its typical concentration in infancy. IgG2, the second largest of IgG isotypes, comprises 20 to 25% of the main subclass and is the prevalent immune response against carbohydrate/polysaccharide antigens. “Adult” concentrations are usually reached by 6 or 7 years old. IgG3 comprises around 5 to 10% of total IgG and plays a major role in the immune responses against protein or polypeptide antigens. The affinity of IgG3 can be higher than that of IgG1. Comprising usually less than 4% of total IgG, IgG4 does not bind to polysaccharides. In the past, testing for IgG4 has been associated with food allergies, and recent studies have shown that elevated serum levels of IgG4 are found in patients suffering from sclerosing pancreatitis, cholangitis and interstitial pneumonia caused by infiltrating IgG4 positive plasma cells.

In certain embodiments, the invention relates to the oligomeric anti-insulin antibody of the invention, wherein the oligomeric anti-insulin antibody is an anti-insulin antibody of the IgM isotype.

As used herein, the term “IgM” has its general meaning in the art and refers to an immunoglobulin that possesses heavy m-chains. Serum IgM exists as a pentamer (or hexamer) in mammals and comprises approximately 10% of normal human serum Ig content. It predominates in primary immune responses to most antigens and is the most efficient complement-fixing immunoglobulin. IgM is also expressed on the plasma membrane of B lymphocytes as membrane-associated immunoglobulin (which can be organized as multiprotein cluster in the membrane). In this form, it is a B-cell antigen receptor, with the H chains each containing an additional hydrophobic domain for anchoring in the membrane. Monomers of serum IgM are bound together by disulfide bonds and a joining (J) chain. Each of the five monomers within the pentamer structure is composed of two light chains (either kappa or lambda) and two heavy chains. Unlike in IgG (and the generalized structure shown above), the heavy chain in IgM monomers is composed of one variable and four constant regions, with the additional constant domain replacing the hinge region. IgM can recognize epitopes on invading microorganisms, leading to cell agglutination. This antibody-antigen immune complex is then destroyed by complement fixation or receptor-mediated endocytosis by macrophages. IgM is the first immunoglobulin class to be synthesized by the neonate and plays a role in the pathogenesis of some autoimmune diseases. Immunoglobulin M is the third most common serum Ig and takes one of two forms: a pentamer (or hexamer under some circumstances) where all heavy chains are identical and all light chains are identical. The membrane-associated form is a monomer (e.g., found on B lymphocytes as B cell receptors) that can form multimeric clusters on the membrane.

IgM is the first antibody built during an immune response. It is responsible for agglutination and cytolytic reactions since in theory, its pentameric structure gives it 10 free antigen-binding sites as well as it possesses a high avidity. Due to conformational constraints among the 10 Fab portions, IgM only has a valence of 5. Additionally, IgM is not as versatile as IgG. However, it is of vital importance in complement activation and agglutination. IgM is predominantly found in the lymph fluid and blood and is a very effective neutralizing agent in the early stages of disease. Elevated levels can be a sign of recent infection or exposure to antigen.

As used herein, the term “IgA” has its general meaning in the art and refers to an immunoglobulin that possesses heavy a-chains. IgA comprises approximately 15% of all immunoglobulins in healthy serum. IgA in serum is mainly monomeric, but in secretions, such as saliva, tears, colostrums, mucus, sweat, and gastric fluid, IgA is found as a dimer connected by a joining peptide. Most IgA is present in secreted form. This is believed to be due to its properties in preventing invading pathogens by attaching and penetrating epithelial surfaces. IgA is a very weak complement-activating antibody; hence, it does not induce bacterial cell lysis via the complement system. However, secretory IgA works together with lysozymes (also present in many secreted fluids), which can hydrolyse carbohydrates in bacterial cell walls thereby enabling the immune system to clear the infection. IgA is predominantly found on epithelial cell surfaces where it acts as a neutralizing antibody. Two IgA subtypes exist in humans, IgA1 und IgA2, while mice have only one subclass. They differ in the molecular mass of the heavy chains and in their concentration in serum. IgA1 comprises approximately 85% of total IgA concentration in serum. Although IgA1 shows a broad resistance against several proteases, there are some that can affect/splice on the hinge region. IgA1 shows a good immune response to protein antigens and, to a lesser degree, polysaccharides and lipopolysaccharides. IgA2, representing only up to 15% of total IgA in serum, plays a crucial role in the mucosa of the airways, eyes and the gastrointestinal tract to fight against polysaccharide and lipopolysaccharide antigens. It also shows good resistance to proteolysis and many bacterial proteases, supporting the importance of IgA2 in fighting bacterial infections.

As used herein, the term “IgD” has its general meaning in the art and refers to an immunoglobulin that possesses heavy d-chains. IgD is an immunoglobulin which makes up about 1% of proteins in the plasma membranes of immature B-lymphocytes where it is usually co-expressed with another cell surface antibody IgM. IgD is also produced in a secreted form that is found in very small amounts in blood serum, representing 0.25% of immunoglobulins in serum. Secreted IgD is produced as a monomeric antibody with two heavy chains of the delta (δ) class, and two Ig light chains.

The term “patient” (or “subject”) as used herein refers to all animals classified as mammals and includes, without limitation, domestic and farm animals, primates and humans, e.g., human beings, non-human primates, cows, horses, pigs, sheep, goats, dogs, cats, or rodents suffering from a disorder or disease. Preferably, the patient is a male or female human of any age or race.

The term “immune-mediated inflammatory disease” or “IMID”, as used herein, refers to any of a group of conditions or diseases that lack a definitive etiology, but which are characterised by common inflammatory pathways leading to inflammation, and which may result from, or be triggered by, a dysregulation of the normal immune response. Because inflammation mediates and is the primary driver of many medical and autoimmune disorders, within the context of the present invention, the term immune-mediated inflammatory disease is also meant to encompass autoimmune disorders and inflammatory diseases.

The term “autoimmune disorder” or “autoimmune disease” refers to a condition in a subject characterised by cellular, tissue and/or organ injury, caused by an immunological reaction of the subject to its own cells, tissues and/or organs. Illustrative, non-limiting examples of autoimmune diseases which can be treated with the methods or pharmaceutical compositions of the invention include alopecia areata, rheumatoid arthritis (RA), ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, sarcoidosis, scleroderma, progressive systemic sclerosis, Sjogren's syndrome, Good pasture's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, Wegener's granulomatosis, anti-glomerular gasement membrane disease, antiphospholipid syndrome, autoimmune diseases of the nervous system, familial mediterranean fever, Lambert-Eaton myasthenic syndrome, sympathetic ophthalmia, polyendocrinopathies, psoriasis, etc.

The term “inflammatory disease” refers to a condition in a subject characterised by inflammation, e.g. chronic inflammation. Illustrative, non-limiting examples of inflammatory disorders include, but are not limited to, Celiac Disease, rheumatoid arthritis (RA), Inflammatory Bowel Disease (IBD), asthma, encephalitis, chronic obstructive pulmonary disease (COPD), inflammatory osteolysis, Crohn's disease, ulcerative colitis, allergic disorders, septic shock, pulmonary fibrosis (e.g., idiopathic pulmonary fibrosis), inflammatory vasculitides (e.g., polyarteritis nodosa, Wegner's granulomatosis, Takayasu's arteritis, temporal arteritis, and lymphomatoid granulomatosis), post-traumatic vascular angioplasty (e.g. restenosis after angioplasty), undifferentiated spondyloarthropathy, undifferentiated arthropathy, arthritis, inflammatory osteolysis, chronic hepatitis, chronic inflammation resulting from chronic viral or bacterial infections, and acute inflammation, such as sepsis.

The term “treat” or “treatment” or “treating”, as used herein, when used directly in reference to a patient or subject shall be taken to mean the administration of a therapy to a patient subject in need of said treatment for the amelioration of one or more symptoms associated with a disease or disorder. Those in need of treatment include those already with the condition or disorder as well as those prone to have the condition or disorder or those in which the condition or disorder is to be prevented. The terms “treat” or “treatment” or “treating” when used directly in reference to damaged tissues shall be taken to mean the amelioration of such damage by both direct mechanisms such as the regeneration of damaged tissues, repair or replacement of damaged tissues (e.g. by scar tissue) as well as through indirect mechanisms e.g., reducing inflammation thereby enabling tissue formation.

In context of the present invention, it is distinguished between monovalent antigenic particles opposed to multivalent antigenic particles. Each particle is considered as a single molecular entity, which may comprise covalently or non-covalently connected portions. However, according to the present invention each particle has an immunogenic activity towards a certain antigen. The monovalent antigen particle is therefore understood to comprise only a single antigenic structure that is able to elicit an immune response to the antigen whereas the multivalent antigen particle comprises multiple copies of such antigenic structure. In context of the present invention sometimes also the terms “soluble” antigen is used for the monovalent antigen particle opposed to “complex” antigen for the polyvalent antigen particle. It is understood that in most instances the antigenic structure comprises or consists of an epitope that elicits an antibody immune response, and in turn is a binding site for an antibody produced upon a cell-mediated immune response as defined herein elsewhere. In other words, the invention distinguishes between a presentation of immune eliciting epitopes as soluble single epitope or in a complexed array identical epitope.

In some embodiments, the invention pertains to a method for treating or preventing a disease which is characterized by the presence of Immunoglobulin G (IgG) type antibodies specific for a disease-associated antigen in a subject, the method comprising administering a therapeutically effective amount of a monovalent antigen particle to the subject, wherein the monovalent antigen particle is composed of an antigenic portion comprising not more than one of an antigenic structure capable of inducing an antibody mediated immune response against the disease-associated antigen.

In an alternative aspect of the invention there is provided a method for treating or preventing a disease which is characterized by the presence of antibodies other than IgG which specific for a disease-associated antigen in a subject, the method comprising administering a therapeutically effective amount of a monovalent antigen particle to the subject, wherein the monovalent antigen particle is composed of an antigenic portion comprising not more than one of an antigenic structure capable of inducing an antibody mediated immune response against the disease-associated antigen. Such disorders of the alternative third aspect can be for example IgE mediated allergies.

A disease which is characterized by the presence of Immunoglobulin G (IgG) type antibodies specific for a disease-associated antigen is preferably a disease characterized by the presence in a subject's serum of pathological IgG molecules, such as autoimmune and alloimmune IgG antibodies. The term “IgG mediated disease” thus includes autoimmune and alloimmune diseases. As used herein, the term “alloimmune disease” refers to when there is a host immune response to foreign antigens of another individual (for example, major or minor histocompatibility alloantigens), for example when there is a host-versus-graft rejection, or alternatively when there is graft-versus-host disease, wherein engrafted immune cells mediate deleterious effects against the host receiving the graft.

In some embodiments, the invention pertains to a monovalent antigen particle for use in treating or preventing a disease which is characterized by the presence of Immunoglobulin G (IgG) type antibodies specific for a disease-associated antigen in a subject, wherein the monovalent antigen particle is composed of an antigenic portion comprising not more than one of an antigenic structure capable of inducing an antibody mediated immune response against the disease-associated antigen.

In this embodiment the above disclosed specific embodiments equally apply here.

In some embodiments, the invention pertains a method for treating or preventing a disease by vaccination in a subject, the method comprising administering an effective amount of a vaccination composition comprising:
(i) a monovalent antigen particle which is composed of an antigenic portion comprising not more than one of an antigenic structure capable of inducing an antibody mediated immune response against a disease-associated antigen, and
(ii) a polyvalent antigen particle which is composed of an antigenic portion comprising more than one of an antigenic structure capable of inducing an antibody mediated immune response against the disease-associated antigen and wherein the more than one of an antigenic structure are covalently or non-covalently cross-linked.

In this embodiment it may be preferred to administer the treatment to the subject in a vaccination scheme that comprises a priming/boosting scheme as disclosed herein elsewhere.

In some embodiments, the invention pertains to vaccination composition for use in treating or preventing a disease in a subject, the vaccination composition comprising:
(i) a monovalent antigen particle which is composed of an antigenic portion comprising not more than one of an antigenic structure capable of inducing an antibody mediated immune response against a disease-associated antigen, and
(ii) a polyvalent antigen particle which is composed of an antigenic portion comprising more than one of an antigenic structure capable of inducing an antibody mediated immune response against the disease-associated antigen and wherein the more than one of an antigenic structure are covalently or non-covalently cross-linked.

In some embodiments, the invention pertains to an immunogenic composition, comprising:
(i) a monovalent antigen particle which is composed of an antigenic portion comprising not more than one of an antigenic structure capable of inducing an antibody mediated immune response against an antigen, and
(ii) a polyvalent antigen particle which is composed of an antigenic portion comprising more than one of an antigenic structure capable of inducing an antibody mediated immune response against the antigen and wherein the more than one of an antigenic structure are covalently or non-covalently cross-linked.

The terms “of the [present] invention”, “in accordance with the invention”, “according to the invention” and the like, as used herein are intended to refer to all aspects and embodiments of the invention described and/or claimed herein.

The methods of the various aspects of the present invention in certain embodiments can be viewed as immunization methods for the generation of certain desired antibody responses in a vertebrate. In this context, preferred embodiments of the inventive methods comprise a priming/boosting immunization scheme of the subject.

The term “priming” an immune response to an antigen refers to the administration to a subject with an immunogenic composition which induces a higher level of an immune response to the antigen upon subsequent administration with the same or a second composition, than the immune response obtained by administration with a single immunogenic composition.

The term “boosting” an immune response to an antigen refers to the administration to a subject with a second, boosting immunogenic composition after the administration of the priming immunogenic composition. In one embodiment, the boosting administration of the immunogenic composition is given about 2 to 27 weeks, preferably 1 to 10 weeks, more preferably 1 to 5 weeks, and most preferably about 3 weeks, after administration of the priming dose.

In a preferred embodiment of the invention the step of priming is performed with the monovalent antigen particle which is composed of an antigenic portion comprising not more than one of an antigenic structure capable of inducing an antibody mediated immune response against the disease-associated antigen, whereas the step of boosting comprises the administration of the polyvalent antigen particle which is composed of an antigenic portion comprising more than one of an antigenic structure capable of inducing an antibody mediated immune response against the disease-associated antigen and wherein the more than one of an antigenic structure are covalently or non-covalently cross-linked. In such priming/boosting embodiment of the invention, the antigenic structure used for inducing the immune response in the priming and the boosting step is the same antigenic structure.

In some embodiments of the invention, the step of boosting may be performed with a combination of monovalent and polyvalent antigen particles as it is described herein.

In some embodiments, the invention pertains to a monospecific IgM-type antibody, or a variant thereof, for use in the treatment of an autoimmune disorder, wherein the monoclonal IgM-type antibody is specific and has a high affinity for an antigen associated with the autoimmune disorder.

In another embodiment, a monospecific IgM-type antibody, or variant thereof, of the invention is not a polyclonal antibody, or the antigen binding fragment is not a fragment of a polyclonal antibody. In more specific embodiments, a monospecific IgM-type antibody, or variant thereof, of the invention is not a primary (polyspecific) IgM-type antibody.

In an alternative, and preferred, embodiment of all monospecific IgM-type antibodies, or variants thereof, of the invention, the monospecific IgM-type antibody, or variant thereof, is an antibody or an antigen binding fragment thereof, and the antibody is a monoclonal antibody, or wherein the antigen binding fragment is a fragment of a monoclonal antibody.

The term “monoclonal antibody” or “mAb” as used herein refers to an antibody obtained from a population of substantially identical antibodies based on their amino acid sequence. Monoclonal antibodies are typically highly specific. Furthermore, in contrast to conventional (polyclonal) antibody preparations which typically include different antibodies directed against different determinants (e.g. epitopes) of an antigen, each mAb is typically directed against a single determinant on the antigen. In addition to their specificity, mAbs are advantageous in that they can be synthesized by cell culture (hybridomas, recombinant cells or the like) uncontaminated by other immunoglobulins. The mAbs herein include for example chimeric, humanized or human antibodies or antibody fragments. In certain embodiments, the invention relates to the oligomeric anti-insulin antibody of the invention, wherein the oligomeric anti-insulin antibody is chimeric, humanized or human.

Monoclonal IgM antibodies in accordance with the present invention may be prepared by methods well known to those skilled in the art. For example, mice, rats, goats, camels, alpacas, llamas or rabbits may be immunized with an antigen of interest (or a nucleic acid encoding an antigen of interest) together with adjuvant. Splenocytes are harvested as a pool from the animals that are administered several immunisations at certain intervals with test bleeds performed to assess for serum antibody titers. Splenocytes are prepared that are either used immediately in fusion experiments or stored in liquid nitrogen for use in future fusions. Fusion experiments are then performed according to the procedure of Stewart & Fuller, J. Immunol. Methods 1989, 123:45-53. Supernatants from wells with growing hybrids are screened by eg enzyme-linked immunosorbent assay (ELISA) for mAb secretors. ELISA-positive cultures are cloned either by limiting dilutions or fluorescence-activated cell sorting, typically resulting in hybridomas established from single colonies. The ability of an antibody, including an antibody fragment or sub-fragment, to bind to a specific antigen can be determined by binding assays known in the art, for example, using the antigen of interest as the binding partner. Alternatively, splenic B cells that bind to the immunizing antigen are sorted as single cells and subsequently the cDNA encoding the heavy and light chain is cloned from single cells. The cloned cDNA is then used for in vitro production of monoclonal recombinant antibodies which are further characterized based on their specificity and affinity to the immunizing antigen.

A monospecific IgM-type antibody, or variant thereof, in accordance with the present invention may be prepared by genetic immunisation methods in which native proteins are expressed in vivo with normal post-transcriptional modifications, avoiding antigen isolation or synthesis. For example, hydrodynamic tail or limb vein delivery of naked plasmid DNA expression vectors can be used to produce the antigen of interest in vivo in mice, rats, and rabbits and thereby induce antigen-specific antibodies (Tang et al, Nature 356: 152 (1992); Tighe et al, Immunol. Today 19: 89 (1998); Bates et al, Biotechniques, 40:199 (2006); Aldevron-Genovac, Freiburg DE). This allows the efficient generation of high-titre, antigen-specific antibodies which may be particularly useful for diagnostic and/or research purposes. For such genetic immunisation, a variety of gene delivery methods can be used, including direct injection of naked plasmid DNA into skeletal muscle, lymph nodes, or the dermis, electroporation, ballistic (gene gun) delivery, and viral vector delivery.

In a further preferred embodiment, a monospecific IgM-type antibody, or variant thereof, of the invention is an antibody or an antigen binding fragment thereof, wherein the antibody is a human antibody a humanised antibody or a chimeric-human antibody, or wherein the antigen binding fragment is a fragment of a human antibody a humanised antibody or a chimeric-human antibody.

Human antibodies can also be derived by in vitro methods. Suitable examples include but are not limited to phage display (CAT, Morphosys, Dyax, Biosite/Medarex, Xoma, Yumab, Symphogen, Alexion, Affimed) and the like. In phage display, a polynucleotide encoding a single Fab or Fv antibody fragment is expressed on the surface of a phage particle (see e.g., Hoogenboom et al., J. Mol. Biol., 227: 381 (1991); Marks et al., J Mol Biol 222: 581 (1991); U.S. Pat. No. 5,885,793). Phage are “screened” to identify those antibody fragments having affinity for target. Thus, certain such processes mimic immune selection through the display of antibody fragment repertoires on the surface of filamentous bacteriophage, and subsequent selection of phage by their binding to target. In certain such procedures, high affinity functional neutralizing antibody fragments are isolated. A complete repertoire of human antibody genes may thus be created by cloning naturally rearranged human V genes from peripheral blood lymphocytes (see, e.g., Mullinax et al., Proc Natl Acad Sci (USA), 87: 8095-8099 (1990)) or by generating fully synthetic or semi-synthetic phage display libraries with human antibody sequences (see Knappik et al 2000; J Mol Biol 296:57; de Kruif et al, 1995; J Mol Biol 248):97).

The antibodies described herein may alternatively be prepared through the utilization of the XenoMouse® technology. Such mice are capable of producing human immunoglobulin molecules and antibodies and are deficient in the production of murine immunoglobulin molecules and antibodies. In particular, a preferred embodiment of transgenic production of mice and antibodies is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000. See also Mendez et al., Nature Genetics, 15:146-156 (1997). Through the use of such technology, fully human monoclonal antibodies to a variety of antigens have been produced. Essentially, XenoMouse® lines of mice are immunized with an antigen of interest. e.g. IGSF11 (VSIG3), lymphatic cells (such as B-cells) are recovered from the hyper-immunized mice, and the recovered lymphocytes are fused with a myeloid-type cell line to prepare immortal hybridoma cell lines. These hybridoma cell lines are screened and selected to identify hybridoma cell lines that produce antibodies specific to the antigen of interest. Other “humanised” mice are also commercially available: eg, Medarex—HuMab mouse, Kymab—Kymouse, Regeneron—Velocimmune mouse, Kirin—TC mouse, Trianni—Trianni mouse, OmniAb—OmniMouse, Harbour Antibodies—H2L2 mouse, Merus—MeMo mouse. Also are available are “humanised” other species: rats: OmniAb—OmniRat, OMT—UniRat. Chicken: OmniAb—OmniChicken.

The term “humanised antibody” according to the present invention refers to immunoglobulin chains or fragments thereof (such as Fab, Fab′, F(ab′)2, Fv, or other antigen-binding sub-sequences of antibodies), which contain minimal sequence (but typically, still at least a portion) derived from non-human immunoglobulin. For the most part, humanised antibodies are human immunoglobulins (the recipient antibody) in which CDR residues of the recipient antibody are replaced by CDR residues from a non-human species immunoglobulin (the donor antibody) such as a mouse, rat or rabbit having the desired specificity, affinity and capacity. As such, at least a portion of the framework sequence of said antibody or fragment thereof may be a human consensus framework sequence. In some instances, Fv framework residues of the human immunoglobulin need to be replaced by the corresponding non-human residues to increase specificity or affinity. Furthermore, humanised antibodies can comprise residues which are found neither in the recipient antibody nor in the imported CDR or framework sequences. These modifications are made to further refine and maximise antibody performance. In general, the humanised antibody will comprise substantially all of at least one, and typically at least two, variable domains, in which all or substantially all of the CDR regions correspond to those of a non-human immunoglobulin and all or substantially all of the framework regions are those of a human immunoglobulin consensus sequence. The humanised antibody optimally also will comprise at least a portion of an immunoglobulin constant region, typically that of a human immunoglobulin, which (eg human) immunoglobulin constant region may be modified (eg by mutations or glycoengineering) to optimise one or more properties of such region and/or to improve the function of the (eg therapeutic) antibody, such as to increase or reduce Fc effector functions or to increase serum half-life. Exemplary such Fc modification (for example, Fc engineering or Fc enhancement) are described elsewhere herein.

The human constant region will most likely be derived from an mu chain sequence, however, any variant thereof, such as Fc region binding attenuated for example gamma chain constant sequences might be used as an IgM variant according to the present invention.

The term “chimeric antibody” according to the present invention refers to an antibody whose light and/or heavy chain genes have been constructed, typically by genetic engineering, from immunoglobulin variable and constant regions which are identical to, or homologous to, corresponding sequences of different species, such as mouse and human. Alternatively, variable region genes derive from a particular antibody class or subclass while the remainder of the chain derives from another antibody class or subclass of the same or a different species. It covers also fragments of such antibodies. For example, a typical therapeutic chimeric antibody is a hybrid protein composed of the variable or antigen-binding domain from a mouse antibody and the constant or effector domain from a human antibody, although other mammalian species may be used.

In particular of such embodiments, a monospecific IgM-type antibody, or variant thereof, of the invention comprises an antigen binding domain of an antibody wherein the antigen binding domain is of a human antibody. Preferably, a monospecific IgM-type antibody, or variant thereof, comprises an antigen binding domain of an antibody or an antigen binding fragment thereof, which is a human antigen binding domain; (ii) the antibody is a monoclonal antibody, or wherein the antigen binding fragment is a fragment of a monoclonal antibody; and (iii) the antibody is a human antibody or a humanised antibody, or wherein the antigen binding fragment is a fragment of a human antibody, a humanised antibody or a chimeric-human antibody.

Light chains of human antibodies generally are classified as kappa and lambda light chains, and each of these contains one variable region and one constant domain. Heavy chains are typically classified as mu, delta, gamma, alpha, or epsilon chains, and these define the antibody's isotype as IgM, IgD, IgG, IgA, and IgE, respectively, as described above. Human IgG has several subtypes, including, but not limited to, lgG1, lgG2, lgG3, and lgG4. Human IgM subtypes include IgM. Human IgA subtypes include lgA1 and lgA2. In humans, the IgA isotypes contain four heavy chains and four light chains; the IgG and IgE isotypes contain two heavy chains and two light chains; and the IgM isotype contains ten or twelve heavy chains and ten or twelve light chains. Antibodies according to the invention may be IgG, IgE, IgD, IgA, or IgM immunoglobulins.

In some embodiments, a monospecific IgM-type antibody, or variant thereof, of the invention is an IgM antibody or fragment thereof. Preferably the antibody of the invention is, comprises or is derived from an IgG immunoglobulin or fragment thereof; such as a human, human-derived IgM immunoglobulin, or a rabbit- or rat-derived IgM.

A monospecific IgM-type antibody, or variant thereof, of the invention, where comprising at least a portion of an immunoglobulin constant region (typically that of a human immunoglobulin) may have such (eg human) immunoglobulin constant region modified—for example eg by glycoengineering or mutations—to optimise one or more properties of such region and/or to improve the function of the (eg therapeutic) antibody, such as to increase or reduce Fc effector functions or to increase serum half-life.

Accordingly, any of the ABPs of the invention described above can be produced with different antibody isotypes or mutant isotypes to control the extent of binding to different Fc-gamma receptors. Antibodies lacking an Fc region (e.g., Fab fragments) lack binding to different Fc-gamma receptors. Selection of isotype also affects binding to different Fc-gamma receptors. The respective affinities of various human IgG isotypes for the three different Fc-gamma receptors, Fc-gamma-RI, Fc-gamma-RII, and Fc-gamma-RIII, have been determined. (See Ravetch & Kinet, Annu. Rev. Immunol. 9, 457 (1991)). Fc-gamma-RI is a high affinity receptor that binds to IgGs in monomeric form, and the latter two are low affinity receptors that bind IgGs only in multimeric form. In general, both IgG1 and IgG3 have significant binding activity to all three receptors, IgG4 to Fc-gamma-RI, and IgG2 to only one type of Fc-gamma-RII called IIaLR (see Parren et al., J. Immunol. 148, 695 (1992). Therefore, human isotype IgG1 is usually selected for stronger binding to Fc-gamma receptors, and IgG2 or IgG4 is usually selected for weaker binding. Preferred embodiments of the invention provide such antibodies where the Fc receptor binding is reduced or eliminated.

A correlation between increased Fc-gamma-R binding with mutated Fc has been demonstrated using targeted cytoxicity cell-based assays (Shields et ah, 2001, J. Biol. Chem. 276:6591-6604; Presta et ah, 2002, Biochem Soc. Trans. 30:487-490). Methods for increasing ADCC activity through specific Fc region mutations include the Fc variants comprising at least one amino acid substitution at a position selected from the group consisting of: 234, 235, 239, 240, 241, 243, 244, 245, 247, 262, 263, 264, 265, 266, 267, 269, 296, 297, 298, 299, 313, 325, 327, 328, 329, 330 and 332, wherein the numbering of the residues in the Fc region is that of the EU index as in Kabat (Kabat et ah, Sequences of Proteins of Immunological Interest (National Institute of Health, Bethesda, Md. 1987).

In certain specific embodiments, said Fc variants comprise at least one substitution selected from the group consisting of L234D, L234E, L234N, L234Q, L234T, L234H, L234Y, L234I, L234V, L234F, L235D, L235S, L235N, L235Q, L235T, L235H, L235Y, L235I, L235V, L235F, S239D, S239E, S239N, S239Q, S239F, S239T, S239H, S239Y, V240I, V240A, V240T, V240M, F241W, F241L, F241Y, F241E, F241R, F243W, F243L, F243Y, F243R, F243Q, P244H, P245A, P247V, P247G, V262I, V262A, V262T, V262E, V263I, V263A, V263T, V263M, V264L, V264I, V264W, V264T, V264R, V264F, V264M, V264Y, V264E, D265G, D265N, D265Q, D265Y, D265F, D265V, D265I, D265L, D265H, D265T, V266I, V266A, V266T, V266M, S267Q, S267L, E269H, E269Y, E269F, E269R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, W313F, N325Q, N325L, N325I, N325D, N325E, N325A, N325T, N325V, N325H, A327N, A327L, L328M, L328D, L328E, L328N, L328Q, L328F, L328I, L328V, L328T, L328H, L328A, P329F, A330L, A330Y, A330V, A330I, A330F, A330R, A330H, I332D, I332E, I332N, I332Q, I332T, I332H, I332Y and I332A, wherein the numbering of the residues in the Fc region is that of the EU index as in Kabat.

Fc variants can also be selected from the group consisting of V264L, V264I, F241W, F241L, F243W, F243L, F241L/F243L/V262I/V264I, F241W/F243W, F241W/F243W/V262A/V264A, F241L/V262I, F243L/V264I, F243L/V262I/V264W, F241Y/F243Y/V262T/V264T, F241E/F243R/V262E/V264R, F241E/F243Q/V262T/V264E, F241R/F243Q/V262T/V264R, F241E/F243Y/V262T/V264R, L328M, L328E, L328F, I332E, L3238M/I332E, P244H, P245A, P247V, W313F, P244H/P245A/P247V, P247G, V264I/I332E, F241E/F243R/V262E/V264R/I332E, F241E/F243Q/V262T/264E/I332E, F241R/F243Q/V262T/V264R/I332E, F241E/F243Y/V262T/V264R/I332E, S298A/I332E, S239E/I332E, S239Q/I332E, S239E, D265G, D265N, S239E/D265G, S239E/D265N, S239E/D265Q, Y296E, Y296Q, T299I, A327N, S267Q/A327S, S267L/A327S, A327L, P329F, A330L, A330Y, I332D, N297S, N297D, N297S/I332E, N297D/I332E, N297E/I332E, D265Y/N297D/I332E, D265Y/N297D/T299L/I332E, D265F/N297E/I332E, L328I/I332E, L328Q/I332E, I332N, I332Q, V264T, V264F, V240I, V263I, V266I, T299A, T299S, T299V, N325Q, N325L, N325I, S239D, S239N, S239F, S239D/I332D, S239D/I332E, S239D/I332N, S239D/I332Q, S239E/I332D, S239E/I332N, S239E/I332Q, S239N/I332D, S239N/I332E, S239N/I332N, S239N/I332Q, S239Q/I332D, S239Q/I332N, S239Q/I332Q, Y296D, Y296N, F241Y/F243Y/V262T/V264T/N297D/I332E, A330Y/I332E, V264I/A330Y/I332E, A330L/I332E, V264I/A330L/I332E, L234D, L234E, L234N, L234Q, L234T, L234H, L234Y, L234I, L234V, L234F, L235D, L235S, L235N, L235Q, L235T, L235H, L235Y, L235I, L235V, L235F, S239T, S239H, S239Y, V240A, V240T, V240M, V263A, V263T, V263M, V264M, V264Y, V266A, V266T, V266M, E269H, E269Y, E269F, E269R, Y296S, Y296T, Y296L, Y296I, A298H, T299H, A330V, A330I, A330F, A330R, A330H, N325D, N325E, N325A, N325T, N325V, N325H, L328D/I332E, L328E/I332E, L328N/I332E, L328Q/I332E, L328V/I332E, L328T/I332E, L328H/I332E, L328I/I332E, L328A, I332T, I332H, I332Y, I332A, S239E/V264I/I332E, S239Q/V264I/I332E, S239E/V264I/A330Y/I332E, S239E/V264I/S298A/A330Y/I332E, S239D/N297D/I332E, S239E/N297D/I332E, S239D/D265V/N297D/I332E, S239D/D265I/N297D/I332E, S239D/D265L/N297D/I332E, S239D/D265F/N297D/I332E, S239D/D265Y/N297D/I332E, S239D/D265H/N297D/I332E, S239D/D265T/N297D/I332E, V264E/N297D/I332E, Y296D/N297D/I332E, Y296E/N297D/I332E, Y296N/N297D/I332E, Y296Q/N297D/I332E, Y296H/N297D/I332E, Y296T/N297D/I332E, N297D/T299V/I332E, N297D/T299I/I332E, N297D/T299L/I332E, N297D/T299F/I332E, N297D/T299H/I332E, N297D/T299E/I332E, N297D/A330Y/I332E, N297D/S298A/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, and S239D/264I/A330L/I332E, wherein the numbering of the residues in the Fc region is that of the EU index as in Kabat. See also WO2004029207, incorporated by reference herein.

In particular embodiments, mutations on, adjacent, or close to sites in the hinge link region (e.g., replacing residues 234, 235, 236 and/or 237 with another residue) can be made, in all of the isotypes, to reduce affinity for Fc-gamma receptors, particularly Fc-gamma-RI receptor (see, eg U.S. Pat. No. 6,624,821). Optionally, positions 234, 236 and/or 237 are substituted with alanine and position 235 with glutamate. (See, eg U.S. Pat. No. 5,624,821.) Position 236 is missing in the human IgG2 isotype. Exemplary segments of amino acids for positions 234, 235 and 237 for human IgG2 are Ala Ala Gly, Val Ala Ala, Ala Ala Ala, Val Glu Ala, and Ala Glu Ala. A preferred combination of mutants is L234A, L235E and G237A, or is L234A, L235A, and G237A for human isotype IgG1. A particular preferred variant of a monospecific IgM-type antibody of the invention is an antibody having human isotype IgG1 and one of these three mutations of the Fc region. Other substitutions that decrease binding to Fc-gamma receptors are an E233P mutation (particularly in mouse IgG1) and D265A (particularly in mouse IgG2a). Other examples of mutations and combinations of mutations reducing Fc and/or C1q binding are E318A/K320A/R322A (particularly in mouse IgG1), L235A/E318A/K320A/K322A (particularly in mouse IgG2a). Similarly, residue 241 (Ser) in human IgG4 can be replaced, eg with proline to disrupt Fc binding.

Additional mutations can be made to a constant region to modulate effector activity. For example, mutations can be made to the IgG1 or IgG2 constant region at A330S, P331S, or both. For IgG4, mutations can be made at E233P, F234V and L235A, with G236 deleted, or any combination thereof. IgG4 can also have one or both of the following mutations S228P and L235E. The use of disrupted constant region sequences to modulate effector function is further described, eg in WO2006118,959 and WO2006036291.

Additional mutations can be made to the constant region of human IgG to modulate effector activity (see, e.g., WO200603291). These include the following substitutions: (i) A327G, A330S, P331S; (ii) E233P, L234V, L235A, G236 deleted; (iii) E233P, L234V, L235A; (iv) E233P, L234V, L235A, G236 deleted, A327G, A330S, P331S; and (v) E233P, L234V, L235A, A327G, A330S, P331S to human IgG1; or in particular, (vi) L234A, L235E, G237A, A330S and P331S (eg, to human IgG1), wherein the numbering of the residues in the Fc region is that of the EU index as in Kabat. See also WO2004029207, incorporated by reference herein.

The affinity of an antibody for the Fc-gamma-R can be altered by mutating certain residues of the heavy chain constant region. For example, disruption of the glycosylation site of human IgG1 can reduce Fc-gamma-R binding, and thus effector function, of the antibody (see, eg WO2006036291). The tripeptide sequences NXS and NXT, where X is any amino acid other than proline, are the enzymatic recognition sites for glycosylation of the N residue. Disruption of any of the tripeptide amino acids, particularly in the CH2 region of IgG, will prevent glycosylation at that site. For example, mutation of N297 of human IgG1 prevents glycosylation and reduces Fc-gamma-R binding to the antibody.

Although activation of ADCC and CDC is often desirable for therapeutic antibodies, there are circumstances in which a monospecific IgM-type antibody, or variant thereof, of the invention is unable to activate effector functions is preferential (eg, an antibodies of the invention that is an agnostic modulator). For these purposes IgG4 has commonly been used but this has fallen out of favour in recent years due the unique ability of this sub-class to undergo Fab-arm exchange, where heavy chains can be swapped between IgG4 in vivo as well as residual ADCC activity. Accordingly, Fc engineering approaches can also be used to determine the key interaction sites for the Fc domain with Fc-gamma receptors and C1q and then mutate these positions, such as in an Fc of a monospecific IgM-type antibody, or variant thereof, of the invention, to reduce or abolish binding. Through alanine scanning Duncan and Winter (1998; Nature 332:738) first isolated the binding site of C1q to a region covering the hinge and upper CH2 of the Fc domain. Researchers at Genmab identified mutants K322A, L234A and L235A, which in combination are sufficient to almost completely abolish Fc-gamma-R and C1q binding (Hezareh et al, 2001; J Virol 75:12161). In a similar manner MedImmune later identified a set of three mutations, L234F/L235E/P331S (dubbed TM), which have a very similar effect (Oganesyan et al, 2008; Acta Crystallographica 64:700). An alternative approach is modification of the glycosylation on asparagine 297 of the Fc domain, which is known to be required for optimal FcR interaction. A loss of binding to Fc-gammaRs has been observed in N297 point mutations (Tao et al, 1989; J Immunol 143:2595), enzymatically degylcosylated Fc domains (Mimura et al, 2001; J Biol Chem 276:45539), recombinantly expressed antibodies in the presence of a glycosylation inhibitor (Walker et al, 1989; Biochem J 259:347) and the expression of Fc domains in bacteria (Mazor et al 2007; Nat Biotechnol 25:563). Accordingly, the invention also includes embodiments of the monospecific IgM-type antibody, or variant thereof, in which such technologies or mutations have been used to reduce effector functions.

IgG naturally persists for a prolonged period in (eg human) serum due to FcRn-mediated recycling, giving it a typical half-life of approximately 21 days. Despite this there have been a number of efforts to engineer the pH dependent interaction of the Fc domain with FcRn to increase affinity at pH 6.0 while retaining minimal binding at pH 7.4. Researchers at PDL BioPharma identified the mutations T250Q/M428L, which resulted in an approximate 2-fold increase in IgG half-life in rhesus monkeys (Hinto et al, 2004; J Biol Chem 279:6213), and researchers at MedImmune have identified mutations M252Y/S254T/T256E (dubbed YTE), which resulted in an approximate 4-fold increase in IgG half-life in cynomolgus monkeys (Dall'Acqua, et al 2006; J Biol Chem 281:23514). A combination of the M252Y/S254T/T256E mutations with point mutations H433K/N434F lead to similar effects (Vaccaro et al., 2005, Nat Biotechnol. October; 23(10):1283-8). ABPs of the invention may also be PEGylated. PEGylation, ie chemical coupling with the synthetic polymer poly-ethylene glycol (PEG), has emerged as an accepted technology for the development of biologics that exercise prolonged action, with around 10 clinically approved protein and peptide drugs to date (Jevsevar et al., 2010; Biotechnol J 5:113). A monospecific IgM-type antibody, or variant thereof, of the invention may also be subjected to PASylation, a biological alternative to PEGylation for extending the plasma half-life of pharmaceutically active proteins (Schlapschy et al, 2013; Protein Eng Des Sel 26:489; XL-protein GmbH, Germany). Similarly, the XTEN half-life extension technology from Amunix provides another biological alternative to PEGylation (Schellenberger, 2009, Nat Biotechnol.; 27(12):1186-90. doi: 10.1038/nbt.1588). Accordingly, the invention also includes embodiments of the antibody in which such technologies or mutations have been used to prolong serum half-life, especially in human serum.

Antibody fragments include “Fab fragments”, which are composed of one constant and one variable domain of each of the heavy and the light chains, held together by the adjacent constant region of the light chain and the first constant domain (CH1) of the heavy chain. These may be formed by protease digestion, e.g. with papain, from conventional antibodies, but similar Fab fragments may also be produced by genetic engineering. Fab fragments include Fab′, Fab and “Fab-SH” (which are Fab fragments containing at least one free sulfhydryl group).

Fab′ fragments differ from Fab fragments in that they contain additional residues at the carboxy terminus of the first constant domain of the heavy chain including one or more cysteines from the antibody hinge region. Fab′ fragments include “Fab′-SH” (which are Fab′ fragments containing at least one free sulfhydryl group).

Further, antibody fragments include F(ab′)2 fragments, which contain two light chains and two heavy chains containing a portion of the constant region between the CH1 and CH2 domains (“hinge region”), such that an interchain disulphide bond is formed between the two heavy chains. A F(ab′)2 fragment thus is composed of two Fab′ fragments that are held together by a disulphide bond between the two heavy chains. F(ab′)2 fragments may be prepared from conventional antibodies by proteolytic cleavage with an enzyme that cleaves below the hinge region, e.g. with pepsin, or by genetic engineering.

An “Fv region” comprises the variable regions from both the heavy and light chains, but lacks the constant regions. “Single-chain antibodies” or “scFv” are Fv molecules in which the heavy and light chain variable regions have been connected by a flexible linker to form a single polypeptide chain, which forms an antigen binding region.

An “Fc region” comprises two heavy chain fragments comprising the CH2 and CH3 domains of an antibody. The two heavy chain fragments are held together by two or more disulphide bonds and by hydrophobic interactions of the CH3 domains.

Accordingly, in some embodiments, the antibodies of the invention is an antibody fragment selected from the list consisting of: Fab′, Fab, Fab′-SH, Fab-SH, Fv, scFv and F(ab′)2.

In a preferred embodiment, an antibody of the invention is an antibody wherein at least a portion of the framework sequence of said antibody or fragment thereof is a human consensus framework sequence, for example, comprises a human germline-encoded framework sequence.

In other certain embodiments, the monospecific IgM-type antibody, or variant thereof, of the invention is modified to prolong serum half-life, especially in human serum. For example, an antibody of the invention may be PEGylated and/or PASylated, or has an Fc region with a T250Q/M428L, H433K/N434F/Y436 or M252Y/S254T/T256E/H433K/N434F modification.

In preferred embodiments, an antibody of the invention can comprise at least one antibody constant domain, in particular wherein at least one antibody constant domain is a CH1, CH2, or CH3 domain, or a combination thereof.

In further of such embodiments, an antibody of the invention having antibody constant domain comprises a mutated Fc region, for example for decreasing interaction of the Fc region with a Fc receptor (Fc receptor on an immune effector cell (eg Saxena & Wu, 2016; Front Immunol 7:580). Examples and embodiments thereof are described elsewhere herein.

In other embodiments, a monospecific IgM-type antibody, or variant thereof, of the invention may comprises an effector group and/or a labelling group. The term “effector group” means any group, in particular one coupled to another molecule such as an antigen binding protein, that acts as a cytotoxic agent. Examples for suitable effector groups are radioisotopes or radionuclides. Other suitable effector groups include toxins, therapeutic groups, or chemotherapeutic groups. Examples of suitable effector groups include calicheamicins, auristatins, geldanamycins, alpha-amanitine, pyrrolobenzodiazepines and maytansines.

The term “label” or “labelling group” refers to any detectable label. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.).

In certain embodiments, the invention relates to the oligomeric anti-insulin antibody of the invention, wherein the immunoglobulin comprises a) a variable heavy (VH) chain comprising CDR1 as defined in SEQ ID NO: 2, CDR2 as defined in SEQ ID NO: 3 and CDR3 as defined in SEQ ID NO: 4 and a variable light (VL) chain comprising CDR1 as defined in SEQ ID NO: 6, CDR2 as defined by the sequence DAS and CDR3 as defined in SEQ ID NO: 7; b) a variable heavy (VH) chain comprising CDR1 as defined in SEQ ID NO: 9, CDR2 as defined in SEQ ID NO: 10 and CDR3 as defined in SEQ ID NO: 11 and a variable light (VL) chain comprising CDR1 as defined in SEQ ID NO: 13, CDR2 as defined by the sequence GAS and CDR3 as defined in SEQ ID NO: 14; or c) a variable heavy (VH) chain comprising CDR1 as defined in SEQ ID NO: 16, CDR2 as defined in SEQ ID NO: 17 and CDR3 as defined in SEQ ID NO: 18 and a variable light (VL) chain comprising CDR1 as defined in SEQ ID NO: 20, CDR2 as defined by the sequence DAS and CDR3 as defined in SEQ ID NO: 21.

In certain embodiments, the invention relates to the oligomeric anti-insulin antibody of the invention, wherein the oligomeric anti-insulin antibody comprises a) comprises a variable heavy (VH) chain sequence comprising the amino acid sequence of SEQ ID NO: 1 or a sequence having at least 80%, 81%, 82%, 83%, 84%, 85%, 86%, 87%, 88%, 89%, 90%, 91%, 92%, 93%, 94%, 95%, 96%, 97%, 98% or 99%, preferably at least 95% sequence identity to SEQ ID NO: 1 and a variable light (VL) chain sequence comprising the amino acid sequence of SEQ ID NO: 4 or a sequence having at least 80%, 81%, 82%, 83%, 84%, 85%, 86%, 87%, 88%, 89%, 90%, 91%, 92%, 93%, 94%, 95%, 96%, 97%, 98% or 99%, preferably at least 95% sequence identity to SEQ ID NO: 4; b) comprises a variable heavy (VH) chain sequence comprising the amino acid sequence of SEQ ID NO: 8 or a sequence having at least 80%, 81%, 82%, 83%, 84%, 85%, 86%, 87%, 88%, 89%, 90%, 91%, 92%, 93%, 94%, 95%, 96%, 97%, 98% or 99%, preferably at least 95% sequence identity to SEQ ID NO: 8 and a variable light (VL) chain sequence comprising the amino acid sequence of SEQ ID NO: 12 or a sequence having at least 80%, 81%, 82%, 83%, 84%, 85%, 86%, 87%, 88%, 89%, 90%, 91%, 92%, 93%, 94%, 95%, 96%, 97%, 98% or 99%, preferably at least 95% sequence identity to SEQ ID NO: 12; or c) comprises a variable heavy (VH) chain sequence comprising the amino acid sequence of SEQ ID NO: 15 or a sequence having at least 80%, 81%, 82%, 83%, 84%, 85%, 86%, 87%, 88%, 89%, 90%, 91%, 92%, 93%, 94%, 95%, 96%, 97%, 98% or 99%, preferably at least 95% sequence identity to SEQ ID NO: 15 and a variable light (VL) chain sequence comprising the amino acid sequence of SEQ ID NO: 19 or a sequence having at least 80%, 81%, 82%, 83%, 84%, 85%, 86%, 87%, 88%, 89%, 90%, 91%, 92%, 93%, 94%, 95%, 96%, 97%, 98% or 99%, preferably at least 95% sequence identity to SEQ ID NO: 19.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

In some embodiments, the oligomeric anti-insulin antibody of the invention comprises a variable light (VL) chain sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 4, SEQ ID NO: 12 or SEQ ID NO:21. In some embodiments, the oligomeric anti-insulin antibody of the invention comprises a variable light (VL) chain sequence having at least 85%, 86%, 87%, 88%, 89%, 90%, 91%, 92%, 93%, 94%, 95%, 96%, 97%, 98%, or 99% sequence identity to the amino acid sequence of SEQ ID NO: 4, SEQ ID NO: 12 or SEQ ID NO:21 and contains substitutions, insertions, or deletions relative to the reference sequence, but retains the ability to bind to insulin and/or proinsulin with high affinity and/or monospecifically. Optionally, the oligomeric anti-insulin antibody of the invention comprises the VL sequence of SEQ ID NO: 4, SEQ ID NO: 12 or SEQ ID NO:21 including post-translational modifications of that sequence.

In certain embodiments, the oligomeric anti-insulin antibody of the invention comprises a variable heavy (VH) chain sequence having at least 85%, 86%, 87%, 88%, 89%, 90%, 91%, 92%, 93%, 94%, 95%, 96%, 97%, 98%, 99% or 100% identity to the amino acid sequence of SEQ ID NO: 1, SEQ ID NO: 8 or SEQ ID NO: 15. In certain embodiments, the oligomeric anti-insulin antibody of the invention comprises a variable heavy (VH) chain sequence having at least 85%, 86%, 87%, 88%, 89%, 90%, 91%, 92%, 93%, 94%, 95%, 96%, 97%, 98%, or 99% identity to the amino acid sequence of SEQ ID NO: 1, SEQ ID NO: 8 or SEQ ID NO: 15 and contains substitutions, insertions, or deletions relative to the reference sequence, but retains the ability to bind to insulin and/or proinsulin with high affinity and/or monospecifically. Optionally, the oligomeric anti-insulin antibody of the invention comprises the VH sequence of SEQ ID NO: 1, SEQ ID NO: 8 or SEQ ID NO: 15, including post-translational modifications of that sequence.

In certain embodiments, a total of 1 to 10 amino acids have been substituted, inserted and/or deleted in SEQ ID NO: 1, SEQ ID NO: 4, SEQ ID NO: 8, SEQ ID NO: 12, SEQ ID NO: 15 and/or SEQ ID NO:21. In certain embodiments, a total of 1 to 5 amino acids have been substituted, inserted and/or deleted in SEQ ID NO: 1, SEQ ID NO: 4, SEQ ID NO: 8, SEQ ID NO: 12, SEQ ID NO: 15 and/or SEQ ID NO:21.

In certain embodiments, substitutions, insertions, or deletions occur in regions outside the CDRs (i.e., in the FRs). In a preferred embodiment, a total of 6 amino acids in SEQ ID NO: 1, SEQ ID NO: 4, SEQ ID NO: 8, SEQ ID NO: 12, SEQ ID NO: 15 and/or SEQ ID NO:21 have been substituted to optimize the expression in mammalian cells.

Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Amino acid substitutions may be introduced into an antibody of interest and the products screened for a desired activity, e.g., retained/improved antigen binding, decreased immunogenicity, or altered ADCC or CDC.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity-matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, 2008, Methods Mol. Biol. 207:179-196), and/or SDRs (a-CDRs), with the resulting variant VH or VL being tested for binding affinity. Affinity maturation by constructing and reselecting from secondary libraries has been described, e.g., in Hoogenboom et al., 2002 in Methods in Molecular Biology 178:1-37. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted. In another embodiment look-through mutagenesis is used to optimize antibody affinity with a multidimensional mutagenesis method that simultaneously assesses and optimizes combinatorial mutations of selected amino acids (Rajpal, Arvind et al., 2005, Proceedings of the National Academy of Sciences of the United States of America vol. 102, 24:8466-71).

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity and/or monospecificity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, 1989, Science, 244: 1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., 1997, TIBTECH 15:26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have an altered influence on inflammation (Irvine, Edward B, and Galit Alter., 2020, Glycobiology vol. 30, 4: 241-253). See, e.g., US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. 2004 J. Mol. Biol. 336:1239-1249; Yamane-Ohnuki et al., 2004, Biotech. Bioeng. 87: 614. Examples of cell lines capable of producing defucosylated antibodies include Lec13 CHO cells deficient in protein fucosylation (Ripka et al., 1986, Arch. Biochem. Biophys. 249:533-545; US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al., 2004, Biotech. Bioeng. 87: 614; Kanda, Y. et al., 2006, Biotechnol. Bioeng., 94(4):680-688; and WO 2003/085107).

Antibodies variants are further provided with bisected oligosaccharides, e.g., in which a biantennary oligosaccharide attached to the Fc region of the antibody is bisected by GlcNAc. Such antibody variants may have altered fucosylation and/or altered influence on inflammation (Irvine, Edward B, and Galit Alter., 2020, Glycobiology vol. 30, 4: 241-253). Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

Antibodies with increased half-lives and improved binding to the neonatal Fc receptor (FcRn), which is responsible for the transfer of maternal IgGs to the fetus (Guyer et al., 1976, J. Immunol. 117:587 and Kirn et al., 1994 J. Immunol. 24:249), are described in US2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (US 2006/0194291).

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In certain embodiments, an antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, the invention relates to a polynucleotide that encodes an oligomeric anti-insulin antibody of the invention.

The term “polynucleotide”, as used herein, refers to a nucleic acid sequence. The nucleic acid sequence may be a DNA or a RNA sequence, preferably the nucleic acid sequence is a DNA sequence. The polynucleotides of the present invention either essentially consist of the aforementioned nucleic acid sequences or comprise the aforementioned nucleic acid sequences. Thus, they may contain further nucleic acid sequences as well. The polynucleotides of the present invention shall be provided, preferably, either as an isolated polynucleotide (i.e. isolated from its natural context) or in genetically modified form. An isolated polynucleotide as referred to herein also encompasses polynucleotides which are present in cellular context other than their natural cellular context, i.e. heterologous polynucleotides. The term polynucleotide encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides.

In certain embodiments, the invention relates to a polynucleotide sequence encoding a variable heavy (VH) chain sequence comprising the nucleotide sequence of SEQ ID NO: 22 or a sequence having at least 85%, 86%, 87%, 88%, 89%, 90%, 91%, 92%, 93%, 94%, 95%, 96%, 97%, 98% or 99% sequence identity to SEQ ID NO: 22, preferably comprising the sequence SEQ ID NO: 23, SEQ ID NO: 24 and SEQ ID NO: 25.

In certain embodiments, the invention relates to a polynucleotide sequence encoding a variable light (VL) chain sequence comprising the nucleotide sequence of SEQ ID NO: 26 or a sequence having at least 85%, 86%, 87%, 88%, 89%, 90%, 91%, 92%, 93%, 94%, 95%, 96%, 97%, 98% or 99% sequence identity to SEQ ID NO: 26, preferably comprising the sequence SEQ ID NO: 27, GATGCATCC and SEQ ID NO: 28.

In certain embodiments, the invention relates to a polynucleotide sequence encoding a) a variable heavy (VH) chain sequence comprising the nucleotide sequence of SEQ ID NO: 22 or a sequence having at least 85%, 86%, 87%, 88%, 89%, 90%, 91%, 92%, 93%, 94%, 95%, 96%, 97%, 98% or 99% sequence identity to SEQ ID NO: 22, preferably comprising the sequence SEQ ID NO: 23, SEQ ID NO: 24 and SEQ ID NO: 25; and b) a variable light (VL) chain sequence comprising the nucleotide sequence of SEQ ID NO: SEQ ID NO: 26 or a sequence having at least 85%, 86%, 87%, 88%, 89%, 90%, 91%, 92%, 93%, 94%, 95%, 96%, 97%, 98% or 99% sequence identity to SEQ ID NO: 26, preferably comprising the sequence SEQ ID NO: 27, GATGCATCC and SEQ ID NO: 28.

In certain embodiments, the invention relates to a polynucleotide sequence encoding a variable heavy (VH) chain sequence comprising the nucleotide sequence of SEQ ID NO: 29 or a sequence having at least 85%, 86%, 87%, 88%, 89%, 90%, 91%, 92%, 93%, 94%, 95%, 96%, 97%, 98% or 99% sequence identity to SEQ ID NO: 29, preferably comprising the sequence SEQ ID NO: 30, SEQ ID NO: 31 and SEQ ID NO: 32.

In certain embodiments, the invention relates to a polynucleotide sequence encoding a variable light (VL) chain sequence comprising the nucleotide sequence of SEQ ID NO: 33 or a sequence having at least 85%, 86%, 87%, 88%, 89%, 90%, 91%, 92%, 93%, 94%, 95%, 96%, 97%, 98% or 99% sequence identity to SEQ ID NO: 33, preferably comprising the sequence SEQ ID NO: 34, GGTGCATCC and SEQ ID NO: 35.

In certain embodiments, the invention relates to a polynucleotide sequence encoding a) a variable heavy (VH) chain sequence comprising the nucleotide sequence of SEQ ID NO: 29 or a sequence having at least 85%, 86%, 87%, 88%, 89%, 90%, 91%, 92%, 93%, 94%, 95%, 96%, 97%, 98% or 99% sequence identity to SEQ ID NO: 29, preferably comprising the sequence SEQ ID NO: 30, SEQ ID NO: 31 and SEQ ID NO: 32; and b) a variable light (VL) chain sequence comprising the nucleotide sequence of SEQ ID NO: 33 or a sequence having at least 85%, 86%, 87%, 88%, 89%, 90%, 91%, 92%, 93%, 94%, 95%, 96%, 97%, 98% or 99% sequence identity to SEQ ID NO: 33, preferably comprising the sequence SEQ ID NO: 34, GGTGCATCC and SEQ ID NO: 35.

In certain embodiments, the invention relates to a polynucleotide sequence encoding a variable heavy (VH) chain sequence comprising the nucleotide sequence of SEQ ID NO: 36 or a sequence having at least 85%, 86%, 87%, 88%, 89%, 90%, 91%, 92%, 93%, 94%, 95%, 96%, 97%, 98% or 99% sequence identity to SEQ ID NO: 36, preferably comprising the sequence SEQ ID NO: 37, SEQ ID NO: 38 and SEQ ID NO: 39.

In certain embodiments, the invention relates to a polynucleotide sequence encoding a variable light (VL) chain sequence comprising the nucleotide sequence of SEQ ID NO: 40 or a sequence having at least 85%, 86%, 87%, 88%, 89%, 90%, 91%, 92%, 93%, 94%, 95%, 96%, 97%, 98% or 99% sequence identity to SEQ ID NO: 40, preferably comprising the sequence SEQ ID NO: 41, GATGCATCC and SEQ ID NO: 42.

In certain embodiments, the invention relates to a polynucleotide sequence encoding a) a variable heavy (VH) chain sequence comprising the nucleotide sequence of SEQ ID NO: 36 or a sequence having at least 85%, 86%, 87%, 88%, 89%, 90%, 91%, 92%, 93%, 94%, 95%, 96%, 97%, 98% or 99% sequence identity to SEQ ID NO: 36, preferably comprising the sequence SEQ ID NO: 37, SEQ ID NO: 38 and SEQ ID NO: 39; and b) a variable light (VL) chain sequence comprising the nucleotide sequence of SEQ ID NO: 40 or a sequence having at least 85%, 86%, 87%, 88%, 89%, 90%, 91%, 92%, 93%, 94%, 95%, 96%, 97%, 98% or 99% sequence identity to SEQ ID NO: 40, preferably comprising the sequence SEQ ID NO: 41, GATGCATCC and SEQ ID NO: 42.

In certain embodiments the polynucleotide encoding an antibody described herein of the invention is suitable for the use as a vector.

In certain embodiments, the invention relates to a host cell comprising the polynucleotide of the invention.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

In certain embodiments the host cell is directly or indirectly used in therapy (e.g., cell therapy). In certain embodiments a method for cell therapy comprises the steps of (i) obtaining a cell from a subject; (ii) transform the cell using a tool (e.g. a vector) comprising the polynucleotide of the invention and/or transform the cell to produce the antibody of the invention; and (iii) administering the transformed cell to a subject. In certain embodiments, the subject in step (i) and step (iii) of the method for cell therapy are the same subject. In certain embodiments, the subject in step (i) and step (iii) of the method for cell therapy are different subjects. In certain embodiments, the subject in step (i) and step (iii) of the method for cell therapy are different subjects that belong to different species. In certain embodiments, the subject in step (i) of the method for cell therapy is a subject from the genus Sus and the subject in step (iii) of the method for cell therapy is a subject from the species Homo sapiens.

In certain embodiments, the host cell is a stem cell. In other embodiments, the host cell is a differentiated cell.

Accordingly, the invention is at least in part based on the surprising finding that the host cell of the invention enables the production of an antibody, variant or fragment that protects and/or regulates the function of a target antigen, in particular of insulin, by competing with the binding of antigen-function limiting antigen-binding agents.

In certain embodiments, the invention relates to a method for producing an oligomeric anti-insulin antibody comprising culturing the host cell of the invention.

In a particular embodiment, the method of producing an antibody comprises culturing the host cell of the invention under conditions suitable to allow efficient production of the antibody of the invention.

In one such embodiment, a host cell comprises (e.g., has been transformed with): (1) a vector comprising a nucleic acid that encodes an amino acid sequence comprising the VL of the antibody and an amino acid sequence comprising the VH of the antibody of the invention, or (2) a first vector comprising a nucleic acid that encodes an amino acid sequence comprising the VL of the antibody and a second vector comprising a nucleic acid that encodes an amino acid sequence comprising the VH of the antibody of the invention. In one embodiment, the host cell is eukaryotic, e.g. a Chinese Hamster Ovary (CHO) cell or lymphoid cell (e.g., YO, NSO, Sp20). In one embodiment, a method of making an antibody, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody according to the invention (e.g. a protective-regulative antibody), nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523; Charlton, 2003, Methods in Molecular Biology, Vol. 248; BKC Lo, 2003, Humana Press, pp. 245-254. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, 2004, Nat. Biotech. 22:1409-1414, and Li et al., 2006, Nat. Biotech. 24:210-215.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177; 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are macaque kidney CVl line transformed by SV40 (COS-7); human embryonic kidney line (293 or 293 cells as described, e.g., in Graham et al., 1997, J. Gen Viral. 36:59); baby hamster kidney cells (BHK); mouse sertoli cells (TM4 cells as described, e.g., in Mather, 1980, Biol. Reprod. 23:243-251); macaque kidney cells (CVl); African green macaque kidney cells (VER0-76); human cervical carcinoma cells (HELA); canine kidney cells (MDCK; buffalo rat liver cells (BRL 3A); human lung cells (Wl38); human liver cells (Hep G2); mouse mammary tumor (MMT 060562); TRI cells, as described, e.g., in Mather et al., 1982, Annals N. Y Aead. Sei. 383:44-68; MRC 5 cells; and FS4 cells. Other useful mammalian host cell lines include Chinese hamster ovary (CHO) cells, including DHFR CHO cells (Urlaub et al., 1980, Proc. Natl. Acad. Sc. USA 77:4216); and myeloma cell lines such as YO, NSO and Sp2/0. For a review of certain mammalian host cell lines suitable for antibody production, see, e.g., Yazaki and Wu, Methods in Molecular Biology, Vol. 248 BKC Lo, 2003, Humana Press, pp. 255-268.

The amount of obtained specific antibody can be quantified using an ELISA, which is also described herein below. Further methods for the production of antibodies are well known in the art, see, e.g. Harlow and Lane, 1988, CSH Press, Cold Spring Harbor.

In certain embodiments, the invention relates to a pharmaceutical composition comprising the oligomeric anti-insulin antibody of the invention and a pharmaceutically acceptable carrier. In certain embodiments, the invention relates to a pharmaceutical composition comprising the polynucleotide of the invention and a pharmaceutically acceptable carrier. In certain embodiments, the invention relates to a pharmaceutical composition comprising the host cell of the invention and a pharmaceutically acceptable carrier.

The term “pharmaceutically acceptable carrier”, as used herein, refers to an ingredient in the composition, other than the active ingredient(s), which is nontoxic to recipients at the dosages and concentrations employed.

Pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US 2005/0260186 and US 2006/0104968.

The pharmaceutically acceptable carrier and/or excipient may facilitate stability, delivery and/or pharmacokinetic/pharmacodynamic properties of the means of the invention.

In certain embodiments, the invention relates to the pharmaceutical composition of the invention comprising a further therapeutic agent.

The term “therapeutic agent”, as used herein, refers to a compound that upon administration to a subject in a therapeutically effective amount, provides a therapeutic benefit to the subject. A therapeutic agent may be any type of drug, medicine, pharmaceutical, hormone, antibiotic, protein, gene, growth factor, bioactive material, used for treating, controlling, or preventing diseases or medical conditions. Those skilled in the art will appreciate that the term “therapeutic agent” is not limited to drugs that have received regulatory approval.

In some embodiments, the therapeutic agent may be selected from the group of a small molecule drug, a protein/polypeptide, an antibody, molecule drug with antibiotic activity, phage-based therapy, a nucleic acid molecule and an siRNA. In some embodiments, the therapeutic agent described herein is a peptide. In some embodiments, the therapeutic agent described herein is a hormone. In some embodiments, the therapeutic agent described herein is insulin.

The inventors demonstrate that the means and methods described herein are useful to regulate endogenous insulin (see e.g. Example 6 and 7). The same mechanism can be used to enhance or protect the effect of therapeutics agents such as therapeutic agents influencing glucose homeostasis e.g. insulin.

Accordingly, the invention is at least in part based on the finding, that the means and methods described herein can improve the effect of other therapeutic agents.

In certain embodiments, the invention relates to the oligomeric anti-insulin antibody of the invention for use in treatment.

In certain embodiments, the invention relates to the polynucleotide of the invention for use in treatment.

In certain embodiments, the invention relates to the host cell of the invention for use in treatment.

In certain embodiments, the invention relates to the pharmaceutical composition of the invention for use in treatment.

In certain embodiments, the invention relates to the oligomeric anti-insulin antibody of the invention, the polynucleotide of the invention, the host cell of the invention, or the pharmaceutical composition of the invention for use in the treatment of an insulin-associated disease or disorder.

The term “insulin-associated disease or disorder”, as used herein, refers to any disease or disorder wherein the insulin production, insulin effect, insulin signaling, insulin distribution, insulin metabolism and/or insulin elimination is dysregulated.

In some embodiments, the insulin-associated disease or disorder is at least one disease or disorder selected from the group of polycystic ovary syndrome, metabolic syndrome and diabetes.

In some embodiments, the insulin-associated disease or disorder is at least one disease or disorder associated with increased levels of at least one agent selected from the group adrenaline, glucagon, cortisol, somatostatin.

In some embodiments, the insulin-associated disease or disorder is at least one side effect of a treatment of an insulin modulating agent. In some embodiments, the insulin modulation agent is selected from the group adrenaline, glucagon, steroid and somatostatin.

The means and methods provided by the invention enable modulation of the immune response against insulin. An immune response against insulin can occur in healthy subjects and/or patients and/or during insulin treatment (see e.g. Example 6 & 7). The inventors show that a broad range of insulin associated symptoms can be influence by the means and methods of the invention (See e.g. FIG. 11, 12, 16 Example 6 & 7). Therefore, the means and methods can improve the effect of administered and/or endogenous insulin and reduce any insulin-associated disease or disorder.

Accordingly, the invention is at least in part based on the surprising finding that the means and methods of the invention can be used to protect and/or regulate insulin function.

In certain embodiments, the invention relates to a method of diagnosing and/or predicting an insulin-associated disease or disorder, the method comprising the steps of:
(i) determining the affinity of the binding of anti-insulin IgM antibodies to proinsulin and/or insulin from a sample, wherein the sample has been obtained from a subject, wherein the subject is diagnosed with an insulin-associated disease or disorder or is at risk thereof; (ii) comparing the level(s) determined in step (i) to a reference value; and (iii) diagnosing and/or predicting an insulin-associated disease or disorder in said subject based on the comparison made in step (ii), preferably wherein a lower affinity of the binding of anti-insulin IgM antibodies to proinsulin and/or insulin indicates a higher risk for an insulin-associated disease or disorder.

The step of determining the affinity of the binding of anti-insulin IgM antibodies to proinsulin and/or insulin from a sample can also be achieved by retrieving the corresponding information from a measurement instrument or from a database.

In certain embodiments, the invention relates to a method for determining whether a subject is susceptible to a treatment of insulin-associated disease or disorder, the method comprising the steps of: (i) determining the affinity of the binding of anti-insulin IgM antibodies to proinsulin and/or insulin from a sample, wherein the sample has been obtained from a subject, wherein the subject is diagnosed with an insulin-associated disease or disorder or is at risk thereof; (ii) comparing the level(s) determined in step (i) to a reference value; and (iii) determining whether said subject is susceptible to the treatment of insulin-associated disease or disorder, preferably wherein a lower affinity of the binding of anti-insulin IgM antibodies to proinsulin and/or insulin indicates a higher susceptibility to the treatment of insulin-associated disease or disorder.

The inventors found that the affinity of the IgM antibody is predictive for disease development, progression and outcome in insulin-associated diseases or disorders (Example 10).

Accordingly, the invention is at least in part based on the predictive information comprised in the state of the IgM antibody affinity of a subject.

In certain embodiments, the invention relates to the oligomeric anti-insulin antibody for use of the invention, the polynucleotide for use of the invention or the host cell for use of the invention, or the pharmaceutical composition for use of the invention, the method of the invention, wherein the insulin-associated disease or disorder is selected from the group of pancreatic damage, type 1 diabetes, type 2 diabetes, exogenous insulin antibody syndrome, gestational diabetes, and dysglycemia.

The term “pancreatic damage”, as described herein, refers to any form of pancreatic abnormality that deregulates insulin production, insulin activity and/or hormones regulating the insulin effect such as adrenaline, glucagon, steroid and somatostatin. In some embodiments, the pancreatic damage described herein is selected from the group of drug-induced pancreatic damage, obesity-induced pancreatic damage and cancer-induced pancreatic damage.

The term “type 1 diabetes”, as used herein, refers to diabetes, primarily characterized by decreased insulin production. Typically type 1 diabetes is characterized by an autoimmune reaction that leads to damage in the insulin producing beta cells of the pancreas.

The term “type 2 diabetes”, as used herein, refers to diabetes primarily characterized by increased insulin resistance. Type 2 diabetes often occurs when levels of insulin are normal or even elevated and appears to result from the inability of tissues to respond appropriately to insulin. Most of the type 2 diabetics are obese.

The term “gestational diabetes”, as used herein, refers to diabetes during pregnancy. gestational diabetes. Symptoms of gestational diabetes additionally includes pregnancy-related symptoms such as preeclampsia and symptoms for the child from a mother with gestational diabetes including, without limitation, growth abnormalities (e.g. macrosomia), impaired glucose homeostasis, jaundice, polycythemia, hypocalcemia, and hypomagnesemia. In some embodiments, the gestational diabetes is diagnosed during pregnancy. In some embodiments, the gestational diabetes is diagnosed before pregnancy.

The term “exogenous insulin antibody syndrome”, as used herein, refers to a hypersensitivity against exogenous insulin and/or insulin resistance associated with circulating insulin antibodies in insulin treated patients.

The term “dysglycemia”, as used herein, refers to an abnormality in blood sugar stability. In some embodiments, the dysglycemia described herein is hypoglycemia. In some embodiments, the dysglycemia described herein is hyperglycemia. In some embodiments dysglycemia is a blood glucose level above 140 mg/dl, 150 mg/dl, 160 mg/dl, 170 mg/dl, 180 mg/dl, 190 mg/dl, 200 mg/dl, 210 mg/dl, or 220 mg/dl 2 hours after glucose intake (typically 75 g glucose) during an oral glucose tolerance test. In some embodiments, dysglycemia is a fasting blood glucose level above 100 mg/dl or 110 mg/dl.

The means and methods described herein can be used to restore deregulated homeostasis insulin and hormones that are influenced by insulin action and/or immune responses against insulin.

Accordingly, the invention is at least in part based on the finding that the means and methods provided herein can restore deregulated homeostasis in various insulin-associated disease or disorder.

In certain embodiments, the invention relates to the oligomeric anti-insulin antibody for use of the invention, the polynucleotide for use of the invention or the host cell for use of the invention, the pharmaceutical composition for use of the invention or the method of the invention, wherein the dysglycemia is dysglycemia in a patient with an insulin-associated disease or disorder is selected from the group of pancreatic damage, type 1 diabetes, type 2 diabetes, exogenous insulin antibody syndrome and gestational diabetes.

In certain embodiments, the invention relates to the oligomeric anti-insulin antibody of the invention for use to enhance the insulin effect. The insulin effect can also be enhanced in patients or in healthy subjects, wherein the insulin effect is regulated by antibodies without necessarily inducing a disease or disorder. For example the composition of the invention, the pharmaceutical product of the invention, the vector of the invention, or the protective-regulative antibody, variant or fragment of the invention, wherein the target antigen is insulin can be used to increase weight gain such as muscle gain. In some embodiments, enhancement of the insulin effect includes, without limitation, increase of glucose uptake, increase of DNA replication, increase of protein synthesis, increased fat synthesis, increased esterification of fatty acids, decreased lipolysis, induction of glycogen synthesis, decreased gluconeogenesis and glycogenolysis, decreased proteolysis, decreased autophagy, increased amino acid uptake, increased blood flow, increase of hydrochloric acid secretion in the stomach, increased potassium uptake, decreased renal sodium excretion.

The means and methods provided by the invention enable modulation of the immune response against insulin. An immune response against insulin can occur in all forms of diabetes and in all forms of insulin treatment. Therefore, the means and methods can improve the effect of administered and/or endogenous insulin and reduce any insulin-deficit related symptom e.g. in diabetes.

Accordingly, the invention is at least in part based on the surprising finding that the means and methods of the invention protect and/or regulate dysregulated insulin function in diabetes.

In certain embodiments, the invention relates to the oligomeric anti-insulin antibody for use of the invention, the polynucleotide for use of the invention or the host cell for use of the invention, or the pharmaceutical composition for use of the invention, the method of the invention, wherein the insulin-associated disease or disorder is diabetes or a symptom thereof.

The term “diabetes”, as used herein, refers to a disease or disorder characterized by hyperglycemia. In some embodiments, diabetes is diagnosed by a glucose level above 140 mg/dl, 150 mg/dl, 160 mg/dl, 170 mg/dl, 180 mg/dl, 190 mg/dl, 200 mg/dl, 210 mg/dl, or 220 mg/dl 2 hours after glucose intake (typically 75 g glucose) during an oral glucose tolerance test. In some embodiments, diabetes is diagnosed by a fasting glucose levels above 100 mg/dl or 110 mg/dl.

Symptoms of diabetes include, without limitation, hyperglycemia, hypoinsulinemia, insulin resistance, polyuria, polydipsia, weight loss, ketoacidosis, glucosuria, fatigue, irritability, blurred vision, slow-healing sores, frequent infections (e.g. gums or skin infections and vaginal infections) and increased inflammation (e.g. chronic-low grade inflammation).

In certain embodiments, the invention relates to a method for producing an oligomeric anti-insulin antibody, preferably of the IgM isotype, comprising immunizing a mammal with a mixture of at least one monovalent insulin particle and at least one polyvalent insulin particle.

The term “insulin particle”, as used herein, refers to an antigen particle (e.g. a poly- or monovalent antigen particle), wherein the antigen is at least partially comprised in insulin and/or proinsulin. In some embodiments the insulin particle comprises an antigen that comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or all amino acids of insulin and/or proinsulin.

In certain embodiments, the invention relates to a method for treatment and/or prevention of an insulin-associated disease or disorder, the method comprising a step of administering a therapeutically effective amount, of the oligomeric anti-insulin antibody of any one of the invention, the polynucleotide of the invention, the host cell of the invention, or the pharmaceutical composition of the invention.

In addition to the above the present invention further relates to the following specific itemized embodiments:

Item 1. A method of eliciting and/or modulating a humoral and/or cell-mediated target antigen-specific immune response in a subject, the method comprising contacting one or more immune-cells of the subject with a combination comprising:
(i) a monovalent antigen particle which is composed of an antigenic portion comprising not more than one of an antigenic structure capable of inducing an antibody mediated immune response against the disease-associated antigen, and
(ii) a polyvalent antigen particle which is composed of an antigenic portion comprising more than one of an antigenic structure capable of inducing an antibody mediated immune response against the disease-associated antigen and wherein the more than one of an antigenic structure are covalently or non-covalently cross-linked.
   Item 2. The method according to item 1, wherein the cell-mediated target antigen-specific immune response involves a lymphocyte, preferably a B lymphocyte (B-cell mediated immune response), preferably which comprises and/or expresses one or more antibody, or variants thereof, and/or B cell receptors, and/or variants thereof, which are specific for the target antigen.
   Item 3. The method of item 1 or 2, wherein the cell-mediated target antigen-specific immune response involves a B cell expressing a Immunoglobulin (Ig) M, IgD, IgA or IgG type antibody and/or B-cell receptor.
   Item 4. The method of any one of items 1 to 3, wherein the more than one of an antigenic structure comprised in the antigenic portion of the polyvalent antigen particle comprises multiple identical antigenic structures.
   Item 5. The method of any one of items 1 to 4, wherein the monovalent-antigen particle further comprises a carrier portion which is coupled to the antigenic portion, optionally via a linker, and wherein the carrier, and optionally the linker, does not comprise another copy of the antigenic structure, and wherein the carrier portion, and optionally the linker, is not capable of eliciting a cell-mediated immune response against the target antigen.
   Item 6. The method of any one of items 1 to 5, wherein the polyvalent-antigen particle further comprises a carrier portion which is coupled to the antigenic portion, optionally via a linker.
   Item 7. The method of item 6, wherein the carrier portion, and optionally the linker, is not capable of eliciting a cell-mediated immune response against the target antigen.
   Item 8. The method of any one of items 5 to 7, wherein the carrier portion is a substance or structure selected from immunogenic or non-immunogenic polypeptides, immune CpG islands, limpet hemocyanin (KLH), tetanus toxoid (TI), cholera toxin subunit B (CTB), bacteria or bacterial ghosts, liposome, chitosome, virosomes, microspheres, dendritic cells, particles, microparticles, nanoparticles, or beads.
   Item 9. The method of any one of items 1 to 8, wherein contacting one or more immune-cells of the subject with a combination comprising a monovalent-antigen particle and a polyvalent-antigen particle involves (i) administration of the monovalent-antigen particle to the subject, (ii) administration of the polyvalent-antigen particle to the subject, or (iii) administration of the monovalent-antigen particle and the polyvalent-antigen particle to the subject, wherein in (i), (ii) and (iii), the immune cells of the subject are as a result of the administration in contact with the combination the monovalent-antigen particle and the polyvalent-antigen particle.
   Item 10. The method of item 9, wherein in (i) the subject is characterized by the presence of the polyvalent-antigen particle before administration of the monovalent-antigen particle, and in (ii) the subject is characterized by the presence of the monovalent-antigen particle before administration of the polyvalent-antigen particle.
   Item 11. The method of any one of items 1 to 10, wherein the combination comprising the monovalent-antigen particle and the polyvalent-antigen particle comprises a specific antigen-ratio monovalent-antigen particle:polyvalent-antigen particle.
   Item 12. The method of item 11, wherein modulating the cell-mediated target antigen-specific immune response in the subject constitutes a reducing of an IgG-type target antigen-specific B-cell response in the subject by contacting one or more of the B-cells of the subject with a combination comprising a specific antigen-ratio which is greater than 1, preferably greater than 10¹, 10², 10³, 10⁴ or more.
   Item 13. The method of item 12, wherein the contacting one or more of the B-cells of the subject with the combination involves administering to the subject an amount of monovalent-antigen particle which is effective to generate in the subject a specific antigen-ratio which is greater than 1, preferably greater than 10¹, 10², 10³, 10⁴ or more.
   Item 14. The method of item 12 or 13, wherein the contacting one or more of the B-cells of the subject with the amount of monovalent-antigen particle is administered either with or without a direct combination of administering polyvalent-antigen particle to the subject.
   Item 15. The method of item 11, wherein modulating the cell-mediated target antigen-specific immune response in the subject constitutes an increasing of an IgG-type target antigen-specific B-cell response in the subject by contacting one or more of the B-cells of the subject with a combination comprising a specific antigen-ration which is less than 1, preferably less than 10⁻¹, 10⁻², 10⁻³, 10⁻⁴ or less.
   Item 16. The method of item 15, wherein the contacting one or more of the B-cells of the subject with the combination involves administering to the subject an amount of polyvalent-antigen particle which is effective to generate in the subject a specific antigen-ratio which is less than 1, preferably less than 10⁻¹, 10⁻², 10⁻³, 10⁻⁴ or less.
   Item 17. The method of item 15 or 16, wherein the contacting one or more of the B-cells of the subject with the amount of polyvalent-antigen particle is administered either with or without a direct combination of administering monovalent-antigen particle to the subject.
   Item 18. The method according to any one of items 1 to 17, wherein the polyvalent-antigen particle comprises the at least two copies of the antigenic structure in spatial proximity to each other, preferably within a nanometer range.
   Item 19. The method of any one of items 1 to 18, wherein the antigen is an autoantigen, a cancer associated antigen, or a pathogen associated antigen.
   Item 20. The method of item 19, wherein the pathogen is selected from a parasite, a monocellular eukaryote, a bacterium, a virus or virion.
   Item 21. The method of any one of items 1 to 20, wherein the antigen is an antigen which is associated with a disease or condition, preferably a disease or condition the subject suffers or is suspected to suffer from.
   Item 22. The method of any one of items 1 to 21, wherein the antigen is a natural or synthetic immunogenic substance, such as a complete, fragment or portion of an immunogenic substance, and wherein the immunogenic substance may be selected from a nucleic acid, a carbohydrate, a peptide, a hapten, or any combination thereof.
   Item 23. The method of any one of the preceding items, wherein the method is for treating a disease or condition in the subject.
   Item 24. The method of item 23, wherein the disease or condition is selected from a disease or condition which is characterized in that an increased or reduced cell-mediated immune response is beneficial for a treatment.
   Item 25. The method of item 23 or 24, wherein the disease or condition is selected from an inflammatory disorder, an autoimmune disease, a proliferative disorder, or an infectious disease.
   Item 26. A method for treating or preventing a disease which is characterized by the presence of Immunoglobulin G (IgG) type antibodies specific for a disease-associated antigen in a subject, the method comprising administering a therapeutically effective amount of a monovalent antigen particle to the subject, wherein the monovalent antigen particle is composed of an antigenic portion comprising not more than one of a antigenic structure capable of inducing an antibody mediated immune response against the disease-associated antigen.
   Item 27. The method of item 26, wherein the disease is an autoimmune disease.
   Item 28. The method of item 26 or 27, wherein the disease-associated antigen is an autoantigen.
   Item 29. The method of any one of items 26 to 28, wherein the disease is characterized by the presence of an endogenous polyvalent antigen particle which is composed of an antigenic portion comprising more than one of a antigenic structure capable of inducing an antibody mediated immune response against the disease-associated antigen and wherein the more than one of a antigenic structures are covalently or non-covalently cross-linked to form a complexed disease-associated antigen structure.
   Item 30. The method of item 29, wherein the therapeutically effective amount of the monovalent antigen particle is an amount that when administered to the subject results in a (serum/tissue) ratio of the administered monovalent antigen particle to the endogenous polyvalent antigen particle of greater than 1.
   Item 31. A method for treating or preventing a disease by vaccination in a subject, the method comprising administering an effective amount of a vaccination composition comprising:
(i) a monovalent antigen particle which is composed of an antigenic portion comprising not more than one of an antigenic structure capable of inducing an antibody mediated immune response against a disease-associated antigen, and
(ii) a polyvalent antigen particle which is composed of an antigenic portion comprising more than one of an antigenic structure capable of inducing an antibody mediated immune response against the disease-associated antigen and wherein the more than one of a antigenic structure are covalently or non-covalently cross-linked.
   Item 32. The method of item 31, wherein disease-associated antigen is a foreign antigen.
   Item 33. The method of item 31 or 32, wherein the vaccination composition comprises a ratio of (i) to (ii) smaller than 1.
   Item 34. An immunogenic composition, comprising:
(i) a monovalent antigen particle which is composed of an antigenic portion comprising not more than one of a antigenic structure capable of inducing an antibody mediated immune response against an antigen, and
(ii) a polyvalent antigen particle which is composed of an antigenic portion comprising more than one of a antigenic structure capable of inducing an antibody mediated immune response against the antigen and wherein the more than one of a antigenic structure are covalently or non-covalently cross-linked.
   Item 35. The immunogenic composition of item 30, wherein the antigenic structure capable of inducing an antibody mediated immune response against the antigen of (i) and (ii) are identical.
   Item 36. The immunogenic composition of item 34 or 35, further comprising a pharmaceutically acceptable carrier and/or excipient.
   Item 37. A monospecific IgM-type antibody, or a variant thereof, for use in the treatment of an autoimmune disorder, wherein the monoclonal IgM-type antibody is specific and has a high affinity for an antigen associated with the autoimmune disorder.
   Item 38. The monospecific IgM-type antibody, or the variant thereof, for use of item 37, wherein the antibody binds to the antigen associated with the autoimmune disorder with a K_{D} of less than 10⁻⁷, preferably of less than 10⁻⁸, more preferably of less than 10⁻⁹ and most preferably of about 10⁻¹⁰.
   Item 39. The monospecific IgM-type antibody, or the variant thereof, for use of item 37 or 38, wherein the monoclonal IgM does not bind to an unrelated antigen, which is an antigen other than the antigen associated with the autoimmune disorder
   Item 40. The monospecific IgM-type antibody, or the variant thereof, for use of any one of items 37 to 39, wherein the treatment does not comprise the use of a polyspecific antibody specific for an unrelated antigen which is an antigen other than the antigen associated with the autoimmune disorder.
   Item 41. The monospecific IgM-type antibody, or variant thereof, for use of any one of items 37 to 40, wherein the variant is a monospecific IgG-type antibody, or a variant thereof, which is Fc attenuated, preferably which is defective for an interaction with Fc-gamma receptors or C1q for use in the treatment of an autoimmune disorder or an alloimmune disorder.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

In particular the individual definitions provided, as well as described specific embodiments in context of one aspect of the invention shall equally apply to the other aspects of the invention.

Unless context dictates otherwise, the descriptions and definitions of the features set out above are not limited to any particular aspect or embodiment of the invention and apply equally to all aspects and embodiments which are described.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

All references, patents, and publications cited herein are hereby incorporated by reference in their entirety.

### BRIEF DESCRIPTION OF THE FIGURES

The figures show:

FIG. 1: shows soluble hapten inhibits antibody immune responses induced by hapten-carrier complexes. a: Schematic wild type B cell expressing IgM (green) and IgD (yellow) B cell receptors. b: Serum anti-NP-Ig titers of NP-KLH immunized (red and green) and CI mice (grey) measured by ELISA at indicated days. Ratios indicated refer to molar ratios of soluble to complex NP (sNP:cNP). Dots represent mice, mean±SD. c: Serum anti-KLH-IgG titers measured by ELISA at indicated days. Dots represent mice, mean±SD. d: ELISpot assay showing NP-specific immunoglobulin producing cells. n=2/group, mean±SD. e: Schematic IgD BCR-knock out B cell. f: Serum anti-NP-Ig titers of NP-KLH immunized (red and green) and CI mice measured by ELISA (IgD−/− mice) at indicated days. Dots represent mice, mean±SD. CI: control immunization.

FIG. 2: shows very high ratios of soluble to complex NP suppress antigen-specific IgM responses. a: Scheme showing 4-Hydroxy-3-Nitrophenylacetyl hapten soluble or conjugated to key hole limpet hemocyanin (KLH). b: Scheme showing immunization schedule with soluble/complex NP and CpG-ODN1826. c: Antibody titers of NP-valency injected mice were analysed via ELISA. Sera were applied in duplicates onto NP-BSA coated plates and diluted in a 1:3 series.

FIG. 3: shows induction of autoantibodies depends on the self-antigen-valency and is modulated by its ratios. a: Scheme of proinsulin-derived full-length CP coupled to KLH carrier. b: Table comparing human to murine CP and Insulin-A chain amino acid sequences. Sequences used as peptides shown underlined, conserved amino acids in bold. c: Schematic immunization schedule. d-e: Serum anti-CP-Ig titers of CP-SAV immunized (red and green) and CI mice (grey) measured by ELISA at indicated days. Boost on d42 was done without CpG (e). Dots represent mice, mean±SD. f: ELISpot assay showing CP-specific immunoglobulin producing spleen-derived cells at d14. Top lane showing representative pictures of wells. n=4 mice/group, mean±SD. g: Serum anti-CP-Ig titers of CP-SAV immunized (red and green) and CI IgD−/− mice (grey) measured by ELISA. Dots represent mice, mean±SD. CP: C-peptide, KLH: key hole limpet hemocyanin, SAV: Streptavidin, CI: control immunization.

FIG. 4: shows soluble antigen interferes with plasma cell differentiation. a: Flow cytometric analysis (FACS) of splenocytes derived from C-peptide (CP) immunized mice. Data representative for two independent experiments (n=4). Ratios on the X-axis refer to molar ratios of monovalent (sCP) to polyvalent (cCP) CP. CD138+ and B220− cells were identified as plasma cells. Top panel showing 0:1 and bottom panel showing 20:1 injected mice. b: Statistical analysis of presented FACS data. Mean+−SD. c: Flow cytometric (FACS) analysis of splenocytes derived from C-peptide (CP) immunized mice. Data representative for two independent experiments (n=4). Ratios on the X-axis refer to molar ratios of monovalent (sCP) to polyvalent (cCP) CP. Top panel showing 0:1 and bottom panel showing 20:1 injected mice. Right panel: quantification. d: Western blot of pancreas lysate with C-peptide (CP) mice sera as primary antibody. Proinsulin (15 kD). e: Streptavidin(carrier)-specific IgG titers of C-peptide (CP) immunized mice were measured via ELISA. Sera of CP:SAV immunized mice were applied onto CP-coated ELISA plates in duplicates and diluted in 1:3 series.

FIG. 5: shows complex native insulin (InsNat) provokes autoreactive IgG responses inducing autoimmune diabetes symptoms in wildtype mice. a: Serum anti-Insulin-Ig titers of InsNat immunized and CI mice measured by ELISA at indicated days. Dots represent mice, mean±SD. b: Flow cytometric analysis of blood showing B cells (CD19+ Thy1.2−) and T cells (Thy1.2+ CD19−) of wildtype (left) and B cell-deficient (right) mice. Cells were pre-gated on lymphocytes. Representative for three independent experiments. c: Blood glucose levels of InsNat immunized (red: WT, yellow: B cell-deficient) and CI mice (grey) were assessed at indicated days post immunization. Dots represent mice, mean±SD. d: Urine glucose levels of InsNat immunized (red) and CI mice (grey) were monitored at indicated days post immunization. Left panel showing visualization of glucose standard (top lane) and representative pictures of tested animals (middle and bottom lanes). Right panel showing quantification. Dots represent mice, mean±SD. e: Water intake of CI and InsNat immunized mice monitored from d21 to d26. f: Flow cytometric analysis of the pancreas of InsNat immunized (red) and CI mice (grey) at day 27. Left panel showing pancreatic macrophages (CD11b+ Ly6G−), neutrophils (Ly6G+ CD11b+) and B cells (CD19+) pre-gated on living cells. Right Panel showing histograms for insulin-binding (top) and streptavidin (SAV)-binding (bottom). Representative for two independent experiments with n=5/group. g: ELISpot of InsNat immunized (red) and CI mice (grey) showing insulin-specific IgG-producing spleen-derived cells (d27). Representative wells are shown (top lane). n=3/group, mean±SD. h: Quantification of total (red) and insulin-specific (salmon) IgG after serum IgG purification of InsNat immunized mice. i: Coomassie stained SDS-page showing purified serum IgG of InsNat immunized (red) and CI mice (grey) under reducing (β-ME), left lanes, and non-reducing conditions, right lanes. HC: heavy chain, LC: light chain. Representative for two independent experiments. j: Blood glucose levels of intravenously (i.v.) injected WT mice. 20 μg of purified serum IgG from InsNat immunized mice (red) or CI mice (grey) at indicated hours post injection. Dots represent mice, mean±SD. CI: control immunization, InsNat: complexed native insulin, β-ME: β-Mercaptoethanol.

FIG. 6: shows an immunization with self-antigen does not alter splenic B cell compartments. a: Flow cytometric analysis of splenocytes derived from InsNat immunized and CI mice. Top panel gating strategy for lymphocytes and single cells single cells. Middle panel showing B cells pre-gated on lymphocytes. Lower panel showing IgM and IgD expression on B cells. Left: Control immunization (CI), right: InsNat immunization (complex native Insulin). n=3/group.

FIG. 7: shows ratios of self-antigen-specific IgM to IgG control the harmfulness of autoimmune reactions and induce protective IgM. a: Serum anti-Insulin-Ig titers of InsA peptide immunized (red and green) and CI mice (grey) measured by ELISA at indicated days. Dots represent mice, mean±SD. b: Blood glucose levels of InsA peptide immunized (red and green) and CI mice (grey) were assessed at indicated days. Dots represent mice, mean±SD. c: Urine glucose levels of InsA peptide immunized (red and green) and CI mice (grey) were monitored at indicated days post immunization. Dots represent mice, mean±SD. d: Ratios of IgG to IgM derived from ELISA values plotted against molar ratios of antigens. n=5/group, mean±SD. e: Western blot analysis of insulin-specific serum IgG derived from InsA peptide immunized mice. Top panel (green): 100:1 serum, lower panel (red): 0:1 serum (sInsA:cInsA). Black filled arrow: Proinsulin (12 kD), Black non-filled arrow: Insulin (6 kD), β-actin (42 kD, loading control). Representative for two independent experiments. f: ELISpot of InsA peptide immunized (red) and CI mice (grey) on d14 showing insulin-specific IgG-producing spleen-derived cells. Representative wells are shown (top lane). n=4/group, mean±SD. g: Ratios of IgG to IgM derived from ELISA values plotted on a two-dimensional graph against blood glucose levels (left panel) and urine glucose levels (right panel). n=5/group, mean±SD. h: Serum anti-Insulin-Ig titers of InsA peptide immunized mice with a γ/μ ratio <0.1 (black) and CI mice (grey) measured by ELISA at indicated days. Dots represent mice, mean±SD. i: Blood glucose levels of InsA peptide immunized mice (γ/μ<0.1; black) and CI mice (grey) were assessed at indicated days post immunization. Dots represent mice, mean±SD. j: Insulin-specific IgM affinity maturation of InsA-peptide immunized mice (left panel) and virus-peptide immunized mice (right panel) at indicated days was measured by ELISA. k: Blood and urine glucose levels of mice immunized with cInsA (red) and cInsA plus pIgM i.v. (salmon). Dots represent mice, mean±SD. CI: control immunization, cInsA: complex Insulin-A peptide.

FIG. 8: shows monovalent soluble virus-derived peptide antigen modulates the IgG versus IgM antibody response induced by corresponding complex antigen. a: Determination of virus-peptide specific serum immunoglobulin titres. Sera of virus-peptide immunized mice were applied onto virus-peptide-bio:Streptavidin (SAV) coated plates in duplicates with 1:3 serial dilution. Mean+−SD. b-c: Determination of KLH(carrier)-specific serum IgG titers. Indicated ratios on the X-axis refers to molecular ratios of soluble to complex virus-peptide. Mean+−SD.

FIG. 9: shows Increased IgMhigh/IgDlow positive compartment upon immunization with autoantigen but not with foreign antigen and pancreatic macrophages binding InsA peptides via IgG. a-b: Flow cytometric analysis of splenocytes derived from virus- or insulin-peptide immunized mice. Top panel (a) showing B cells (CD19+ B220+) pre-gated on lymphocytes. Lower panel (b) showing B cell subsets: mature B cells (IgDhi IgMlo), transitional/marginal zone B cells (IgDlo IgMhi). Cells were pre-gated on B cells. Left: PBS (grey), middle: Virus-peptide (grape), right: Insulin-peptide (teal). Outer right shows quantification, mean+−SD. c: Flow cytometric analysis of pancreatic cells. Left panel showing gating strategy for cells (top) and Macrophages (bottom). Right panel showing histograms for InsA-peptide and peptide control binding as indicated.

FIG. 10: shows splenic macrophages bind insulin-specific IgG in cInsA-peptide immunized mice. a: Flow cytometric analysis (FACS) of splenocytes of cInsA-peptide immunized mice. Left panel showing gating strategy for macrophages (CD11b+ CD19−). Top panel showing IgG binding histograms of control immunization (black) and cInsA-immunized (red) mice. Lower panel showing InsA-peptide binding of macrophages. Representative data for three independent experiments.

FIG. 11: shows dysregulated glucose metabolism is prevented by increasing IgM upon repeated re-challenge with cInsA complexes. a: Determination of Insulin-specific serum immunoglobulin titres. Sera of InsA-peptide immunized mice were applied in duplicates onto native Insulin coated ELISA plates in 1:3 serial dilution. Left panel showing anti-Insulin IgM on d49, right panel showing anti-Insulin IgG in arbitrary units (AU). Indicated ratios on the X-axis refers to molecular ratios of soluble to complex InsA-peptide. Mean+−SD. b: Urine glucose levels were monitored by test stripes. Mean+−SD.

FIG. 12: shows polyreactive IgM induced by InsA peptide immunization leads to diabetes symptoms depending on the antigen valence and day. a: Blood glucose levels were monitored by AccuCheck system (Roche). Freshly drawled blood from the tail vein was applied onto test stripes and blood glucose was measured in mmol/L. Mean+−SD. b: Urine glucose levels were monitored by Combur M stripes (Roche). Freshly obtained urine was applied onto the glucose fields of test stripes and analysed according to manufacturer's standard. Green bars indicate 100:1 (soluble:complex) InsA-peptides. Mean+−SD. Dots represent mice used in this study.

FIG. 13: shows generation of autoreactive IgM by increased ratio of monovalent antigen (100:1, sInsA:cInsA) protects from dysregulated glucose metabolism induced by complex antigen (0:1, sInsA:cInsA). a: Blood glucose levels were monitored by AccuCheck system (Roche). Freshly drawled blood from the tail vein was applied onto test stripes and blood glucose was measured in mmol/L. Mean+−SD. b: Urine glucose levels were monitored by Combur M stripes (Roche). Freshly obtained urine was applied onto the glucose fields of test stripes and analysed according to manufacturer's standard. Green bars indicate 100:1 (soluble:complex) InsA-peptides. Mean+−SD. Dots represent mice. c: Determination of Insulin-specific serum immunoglobulin titers. Sera of InsA-peptide immunized mice were applied in duplicates onto native Insulin coated ELISA plates in 1:3 serial dilution. (a) showing anti-Insulin IgM on d59, whereas (b) showing anti-Insulin IgG in arbitrary units (AU). Indicated ratios on the X-axis refer to molecular ratios of soluble to complex InsA-peptide. Mean+−SD.

FIG. 14: shows repeated re-challenge with cInsA complexes results in accumulation of insulin-specific IgM+ B cells. a: Flow cytometric analysis (FACS) of splenocytes (d79) of cInsA immunized (d71) WT mice. Left panel showing forward and sideward scatter with lymphocyte gating. Middle panel pre-gated on lymphocytes shows B cells (CD19+ B220+). Right panel pre-gated on B cells shows histogram of InsA-peptide binding. Red: g/μ<0.1; black: g/μ<0.1 SAV only control.

FIG. 15: shows Intravenous administration of purified serum pIgM does not lead to autoimmune dysglycemia. a: Coomassie stained SDS-page showing purified serum IgM of InsA peptide (d49) immunized (red) and CI mice (grey) under reducing (b-ME), left lanes, and non-reducing conditions, right lanes. HC: heavy chain, LC: light chain. Representative for two independent experiments. b-c: Blood glucose levels of intravenously injected mice with either 20 μg CI IgM (grey) or InsA IgM (black). Dots represent mice, mean±SD. CI: control immunization, pIgM: protective IgM. d: anti-KLH-IgM serum titers measured by ELISA.

FIG. 16: shows differences in the affinity and specificity of primary versus memory IgM control autoimmune responses. a: Schematic illustration of immunization schedule with complex Ins-A-peptides (cInsA) intraperitoneally and insulin-specific protective IgM (PR-IgM) in 48 hours cycles intravenously (i.v.). *monitoring: diabetes symptoms were only observed within cInsA only group. b: Blood and urine glucose levels of wild-type mice on day 7 immunized with complex InsA-peptides (cInsA) (red, n=5) and cInsA plus intravenously injected (i.v.) pIgM (salmon, n=5). Dots represent individual mice, mean±SD. c: Serum anti-dsDNA-IgM titers of Insulin-A-peptide immunized mice on day 7 (n=8) and day 85 (n=4) measured by ELISA. Dots represent individual mice, mean±SD. d, f: Serum anti-nuclear-IgM (ANA) of control-immunized (CI, n=3), Insulin-A-peptide immunized mice on day 7 (n=3) and day 85 (n=3) with total serum or Insulin-specific IgM (Isotype control: n=3, day 7: n=3, day 85: n=3) analyzed via HEp-2 slides. Scale bar: 10 μm. Green fluorescence indicates IgM bound to nuclear structures e: Coomassie stained SDS-page showing primary (cInsA d7) and memory (cInsA d85) Insulin-specific IgM after incubation with Insulin/DNA and size exclusion with a cut-off at 10,000 kD (referring to >/<104 kD). IgM heavy chain: 69 kD, IgM light chain: 25 kD, J-segment: 15 kD. Data presented are representative of three independent experiments. g: Blood glucose levels of wild-type mice intravenously injected with either IgM isotype ctrl (grey, n=6), memory PR-IgM (black, protective Insulin-IgM d85, n=5), or primary Insulin-IgM (red, d7, n=4) after Insulin-pulldown. mean±SD. Statistical analysis compares red line time points with black line time points.

FIG. 17: shows insulin-specific pulldown of sera of cInsA immunized mice contains Insulin-reactive IgM. a: Western blot analysis of Insulin-specific pulldown of cInsA immunized mice sera. CI: control immunization. Top panel (green) shows IgM heavy chain (IgM HC, 69 kD) and bottom panel shows IgG heavy chain (IgG HC, 55 kD). b: Serum IgM of control immunized mice against DNA (left) and Insulin (right) measured via ELISA. Mean+−SD. Dots represent individual mice.

FIG. 18: shows a graphical summary in the case of insulin. Responsiveness of insulin-specific B cells is controlled by antigen-valences leading to inducible protective autoreactive IgM under physiological conditions. pIgM: protective IgM, sInsulin: soluble (monovalent), cInsulin: complex (multivalent).

FIG. 19: Antibody responses after immunization with SARS-CoV-2-derived RBD. Mice were pre-treated as indicated two weeks before immunization. Subsequently, the mice were immunized at day 1 and day 21. Serum was collected at day 28 after immunization concentrations and used in ELISA to determine Ig concentration.

FIG. 20: Immunization of mice with cInsulin induces acute inflammatory pancreatitis.
A) FACS measurement showing germinal center B cells that bind native Insulin
B) ELISA measurement showing serum pancreatic lipase which was used as marker for pancreas damage. In agreement with the autoimmune reaction induced by polyvalent Insulin, a remarkable increase in serum pancreatic lipase was detected as a clear sign for organ damage.
C) Competition assay for insulin binding to IgM. Serum of wild-type mice immunized with cInsA was preincubated either with BSA (untreated control, UT) or with 50 μg/mLcalf-thymus dsDNA (+DNA). Data show the relative reduction in insulin binding to primary IgM (d7) after preincubation with dsDNA suggesting that dsDNA competes with insulin for binding to primary IgM, which is, in contrast to PR-IgM, poly-specific
D) Quantitative data for the affinity measurements Interferometric assay for direct Insulin:IgM interactions showing differences in the affinities of primary IgM compared with PR-IgM.
E) Flow cytometry-based bead array of pancreas supernatant of mice immunized with cInsulin (n=3) or control immunization (n=3). Representative histograms of cytokine beads (left) and cytokine detection (right).
F) Quantitative data for the affinity measurements. Interferometric assay for direct Insulin:IgM interactions showing differences in the affinities of primary IgM compared with PR-IgM.

FIG. 21: Autoantibodies are required to balance homeostasis in mice.
A: Insulin-specific IgG concentrations of different IgG pulldowns measured via ELISA (coating: native Insulin). Total: total IgG pulldown via protein G (n=5), Insulin-specific: IgG pulldown via Insulin bait column (n=5), control IgG (n=3). B: Coomassie stained SDS page showing total IgG (pulldown from serum) and IgG control (total IgG depleted for anti-Insulin-IgG). Presented image is representative of three independent experiments. Marker on the left is shown in kilodaltons (kD). C: Anti-Insulin-IgG secreting splenocytes of naïve wildtype and B cell-deficient (B cell-def) mice measured by ELISpot (coating: native Insulin). Cells were seeded at 300,000 cells/well and incubated for 48 hours. D: Blood glucose levels of naïve wildtype and B cell deficient mice measured with a commercial blood glucose monitor (mmol/L). E: Blood glucose levels of wildtype and B cell deficient mice intravenously injected with 200 μg total IgG, IgG depleted for anti-Insulin-IgG measured at indicated hours. F: Motor function of wildtype (WT) and B cell-deficient (B cell-def) mice as measured by wire hanging test (in on-wire seconds). Grey: WT untreated, blue: B cell-def untreated, green: B cell-def injected with 200 μg total IgG. G: Insulin titers of B cell-deficient (B cell-def) mice injected with 100 μg commercial human IVIg as measured by ELISA at indicated time points. H: Blood glucose levels of wildtype mice injected with 200 μg commercial human IVIg (black) and commercial human IVIg depleted for anti-Insulin-IgG (grey) measured by a commercial blood glucose monitor (mmol/L) at indicated hours. I: Serum glucose levels of immunodeficiency patients (common variable immune deficiency, CVID) that received (500 mg/kg) IVIg before (pre) and after (post) treatment compared to healthy donor (HD) controls.
J: Insulin-binding affinity of human anti-insulin-IgG determined by bio-layer interferometry (BLI). The Kd (dissociation constant) was calculated by using the Ka (association constant): 1/Ka. Shown data are representative for three independent experiments.

FIG. 22 Neutralizing and PR-IgM exists in humans.
A: Serum anti-Insulin-IgM concentrations of young (<30 years) and old (>65 years) individuals measured via ELISA (coating: native Insulin). Women (young): n=25, women (old): n=11, men (young): n=15, men (old): n=12. Mean, ±SD, statistical significance was calculated using Kruskal-Wallis-test. B: Scheme showing column-based purification of insulin-specific IgM fractionated into low and high affinity fractions. C: Coomassie stained SDS page showing low-affinity anti-Insulin IgM (red) and high-affinity anti-Insulin-IgM (green) after purification. Presented image is representative of three independent experiments. Marker on the left is shown in kilodaltons (kD), HC (heavy chain): 70 kD, LC (light chain): 25 kD, J (J-segment): 15 kD. D: HEp2 slides showing anti-DNA-reactive IgM of insulin-specific IgM pulldowns. Black: monoclonal IgM control (n=6), red: low-affinity anti-Insulin IgM (n=6), green: high-affinity anti-Insulin IgM (n=6). Scale bar: 10 μm. Green fluorescence indicates HEp2 cell binding. Images representative of three independent experiments. E: Anti-dsDNA-IgM concentration of insulin-specific IgM pulldowns as measured by ELISA (coating: calf-thymus DNA). IgM control (ctrl, n=3), IgMlow (n=3), IgMhigh (n=3). Mean, ±SD, statistical significance was calculated using Kruskal-Wallis-test. F: Insulin-binding affinity of human anti-insulin-IgM pulldowns determined by bio-layer interferometry (BLI). The Kd (dissociation constant) was calculated by using the Ka (association constant): 1/Ka. Shown data are representative for three independent experiments. Uppercase letter refers to affinity fractions. G: Blood glucose levels of wildtype mice intravenously injected with 100 μg human insulin-specific IgM (uppercase refers to affinity fraction) and human IgM control. H, I: Blood glucose levels of wildtype mice intravenously injected with 100 μg human insulin-specific IgM (uppercase refers to affinity fraction) and human IgM control together with 500 ng native Insulin (H) and together with 100 μg human anti-Insulin-IgG (I). J: Ratio of insulin-specific IgM of young (<30 years) and old (>65 years) individuals as determined by ELISA. Insulin-specific IgM was isolated via insulin-bait columns before experiments.

FIG. 23 Endogenous Insulin complexes induce robust autoimmunity in mice.
A: Schematic illustration of insulin tetramers (cInsulin) generated by thiol group mediated disulfide crosslinking via 1,2-phenylene-bis-maleimide. Black lines: endogenous disulfide bonds, red lines: induced disulfide bonds. B: Coomassie stained SDS page showing Insulin (left lane) and crosslinked insulin (right lane; left panel) and cInsulin complexes after purification with a 10 kD size exclusion column (right panel). Presented images are representative of three independent experiments. Marker on the left is shown in kilodaltons (kD). C: Blood glucose levels of wildtype mice intraperitoneally injected with PBS (control injection; CI, n=5), cInsulin (n=5), Insulin: SAV (n=5) on day 0. Mean, ±SD, statistical significance was calculated using repeated measure ANOVA test. D: Serum anti-Insulin-IgM concentrations of wildtype mice intraperitoneally injected with PBS (control injection; CI, n=5) and cInsulin (n=3) on day 0 measured by ELISA at indicated days (coating: native Insulin). Mean, ±SD, statistical significance was calculated using Kruskal-Wallis-test. E: Blood glucose levels of wildtype mice intraperitoneally injected with PBS (control injection; CI, n=5) and cInsulin (n=5) on day 0 and day 21 followed by intravenous injections of 100 μg anti-Insulin IgM (high affinity) or 100 μg IgM ctrl on day 22. F: Flow cytometric analysis of mice intraperitoneally injected with PBS (n=5) and cInsulin (n=5/group) together with intravenous 100 μg anti-Insulin-IgM (high-affinity) or 100 μg IgM control. Panels show pancreatic macrophages (CD11b+) and neutrophils (Ly6G+) pre-gated on viable cells. Images are representative of three independent experiments. G: Serum pancreatic lipase levels of wildtype mice intraperitoneally injected with PBS (n=5) and cInsulin (n=5/group) together with intravenous 100 μg anti-Insulin-IgM (high-affinity) or 100 μg IgM control. H: Schematic illustration of the macrophage assay used to assess phagocytosis activity. I: Flow cytometric analysis of bead-based phagocytosis assay performed with high or low affinity murine anti-Insulin-IgM. Left panel shows representative FACS plots for the percentage of phagocytosing macrophages in the presence of low or high affinity IgM. Right panel show quantitative analysis for the percentage of phagocytosing macrophages.

FIG. 24 Monoclonal human insulin-IgM is able to protect Insulin in vivo.
A: Coomassie stained SDS page showing monoclonal anti-Insulin-IgM and IgG after purification. Presented image is representative of three independent experiments. Marker on the left is shown in kilodaltons (kD). B: Insulin-binding affinity of monoclonal human anti-insulin-Ig determined by bio-layer interferometry (BLI). The Kd (dissociation constant) was calculated by using the Ka (association constant): 1/Ka. Shown data are representative for three independent experiments. C: Anti-dsDNA-IgM concentration of insulin-specific IgM pulldowns as measured by ELISA (coating: calf-thymus DNA). IgM control (ctrl, n=4), IgMMY (n=4), IgGMY (n=4). D: HEp2 slides showing anti-DNA-reactive monoclonal IgMMY (n=6) and IgGMY (n=6). Scale bar: 10 μm. Green fluorescence indicates HEp2 cell binding. Images representative of three independent experiments. E: Blood glucose levels of wildtype mice intraperitoneally injected with PBS (control injection; CI, n=5) and cInsulin (n=5) on day 0 and day 21 followed by intravenous injections of 100 μg anti-Insulin IgM (high affinity) or 100 μg IgM ctrl on day 22. F: Blood glucose levels of wildtype mice intraperitoneally injected with PBS (control injection; CI, n=5) and cInsulin (n=5) on day 0 and day 21 followed by intravenous injections of 100 μg anti-Insulin IgM (high affinity) or 100 μg IgM ctrl on day 22. G: Urine glucose levels of wildtype mice intraperitoneally injected with PBS (control injection; CI, n=5) and cInsulin (n=5) on day 0 and day 21 followed by intravenous injections of 100 μg anti-Insulin IgM (high affinity) or 100 μg IgM ctrl on day 22.

FIG. 25 No antibody secreting cells in mbi-deficient mice.
A: Flow cytometric analysis of blood of wild-type and B cell-deficient mice. Left panel showing cells in forward and sideward scatter. Middle and right panel showing cells pre-gated on lymphocytes.
B: IgG secreting splenocytes of wild-type and B cell-deficient mice measured by ELISpot. 50,000 splenocytes were seeded per well.
C, D: Serum total IgG (C) and total IgM (D) titers of wild-type and B cell deficient mice as measured by ELISA.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### Example 1: Immunization Experiments and Antibody Response

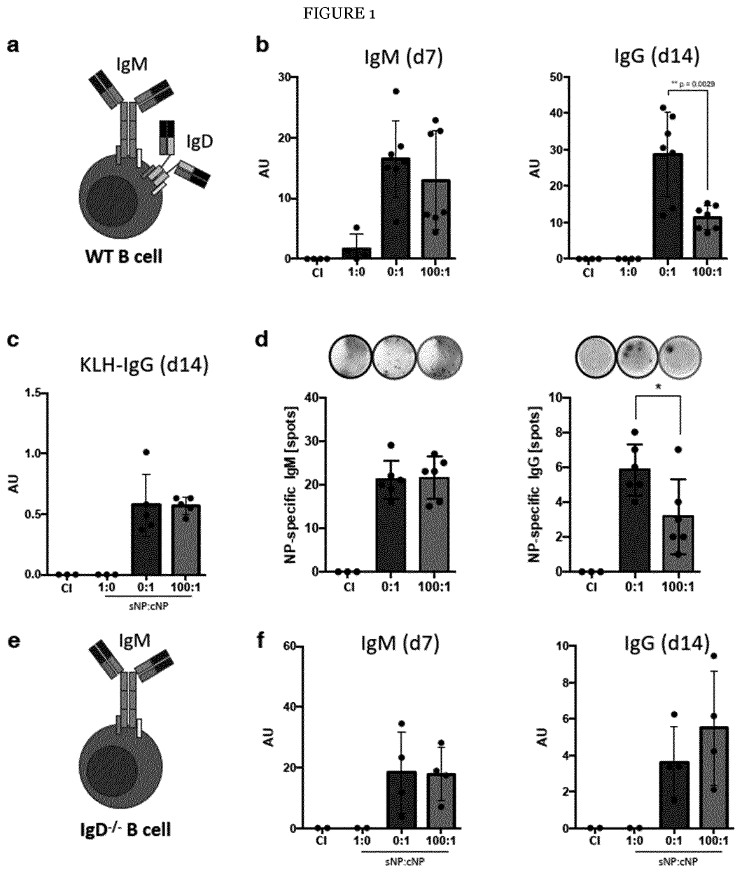

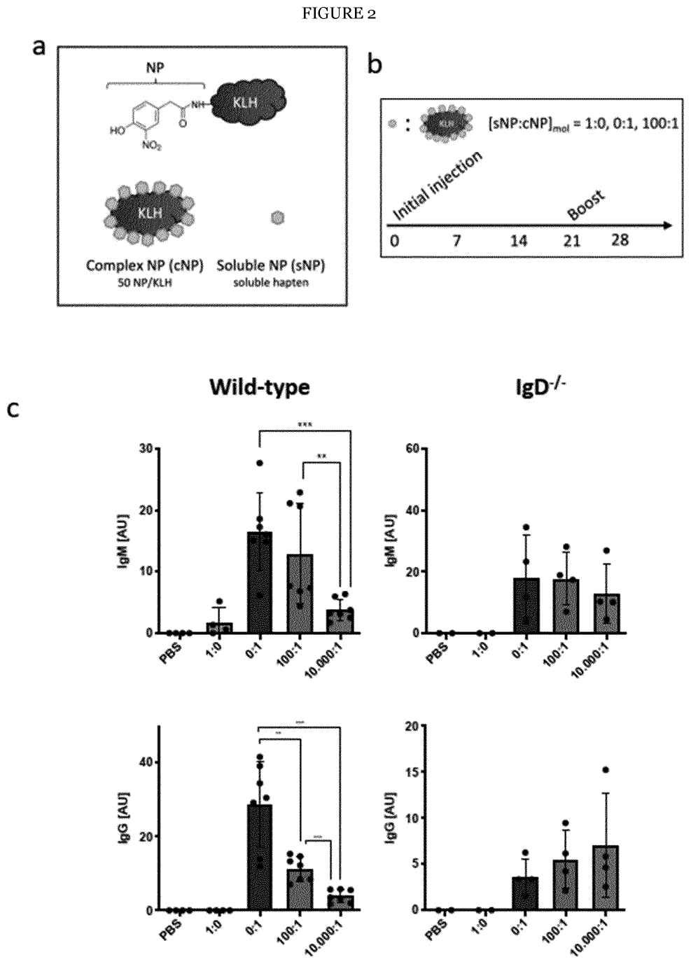

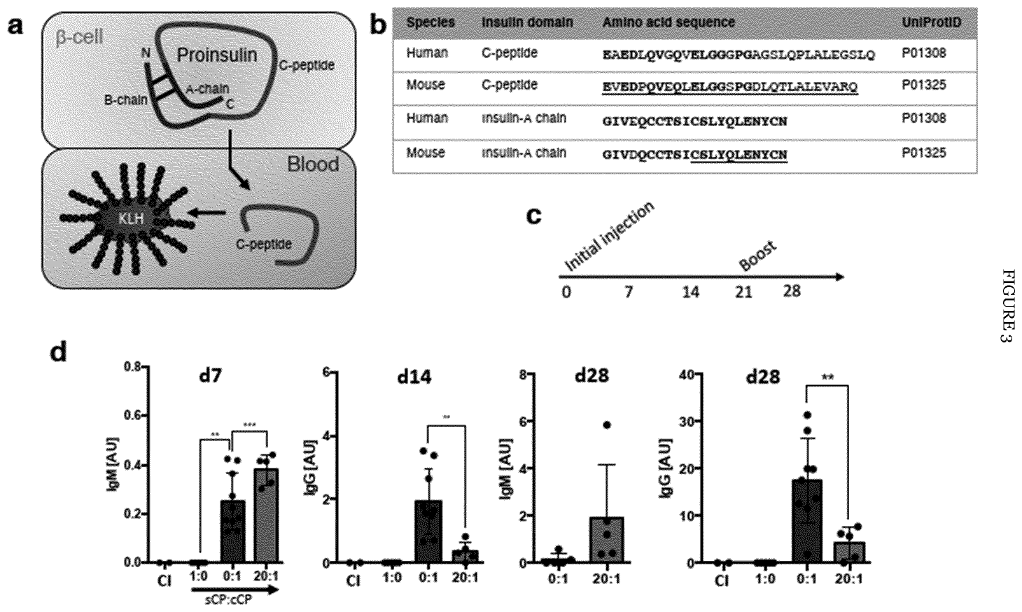

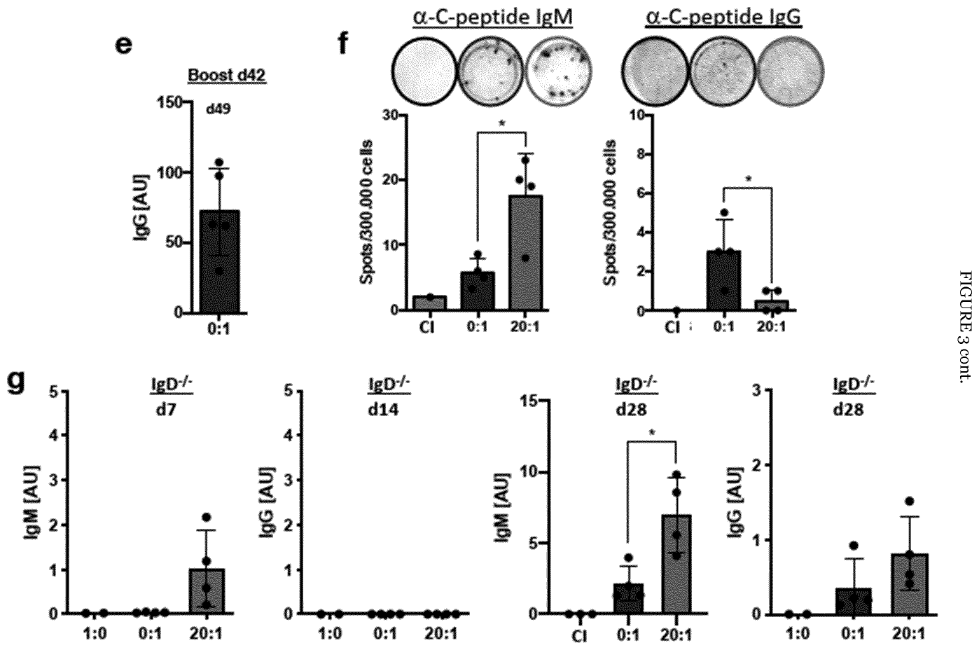

The presence of soluble hapten suppresses IgG production: To test the concept of relative responsiveness of B cells in vivo, immunization experiments were performed using NP (4-hydroxy-3-nitrophenylacetyl) as hapten coupled to KLH (Keyhole Limpet Hemocyanin) as carrier (FIGS. 2a and b). To this end, groups of wild-type mice were injected with either NP as soluble compound (sNP) or NP-KLH, referred to as multivalent complex antigen (cNP), at equal molar ratios for NP (FIG. 1a). Antibody responses were determined at day 7 (IgM) and day 14 (IgG) post immunization (FIG. 1b). Similar to control immunization (CI) lacking the studied antigen (CI), injection of only soluble hapten (sNP:cNP, 1:0) failed to induce clear IgM or IgG antibody responses, while injection of cNP as multivalent antigen (sNP:cNP, 0:1) was able to induce both. Adding sNP to cNP at different molar ratios interfered with antibody responses. Interestingly, the IgG response was significantly impeded at already 100:1 ratio for sNP to cNP. Using higher ratios of sNP to cNP (>10,000:1) was also able to significantly repress the IgM antibody response to NP hapten (FIG. 2c). Importantly, the IgG response to the carrier (KLH) was similar regardless of the amount of soluble hapten (FIG. 1c).

To further confirm these findings, ELISpot assays were performed to directly assess the ratio of antibody secreting cells. In agreement with the serum immunoglobulin data, the ELISpot results showed that combining the soluble hapten with hapten-coupled carrier at 100:1 ratio reduces the number of IgG secreting cells while IgM secreting cells are unaffected (FIG. 1d). These data are in agreement with the inventors' concept that soluble monovalent antigen inhibits immune response to complex forms of the same antigen. In contrast to IgM, the inhibitory effect on IgG immune responses is observed at lower concentrations of the soluble monovalent antigen.

An important part, it was suggested that the presence of IgD-type BCR is important for this regulation. Thus, tested the role of IgD was tested by conducting the NP immunization experiments in IgD knockout mice lacking IgD-type BCR. The IgD knockout mice showed no inhibitory effects when soluble NP was added to cNP immunization (FIG. 1e, f; FIG. 2c).

Together, these data suggest that mature B cells are able to fine-tune their immune response according to the density of antigenic determinants thereby leading to distinct IgM and IgG responses to different epitopes of the same antigen.

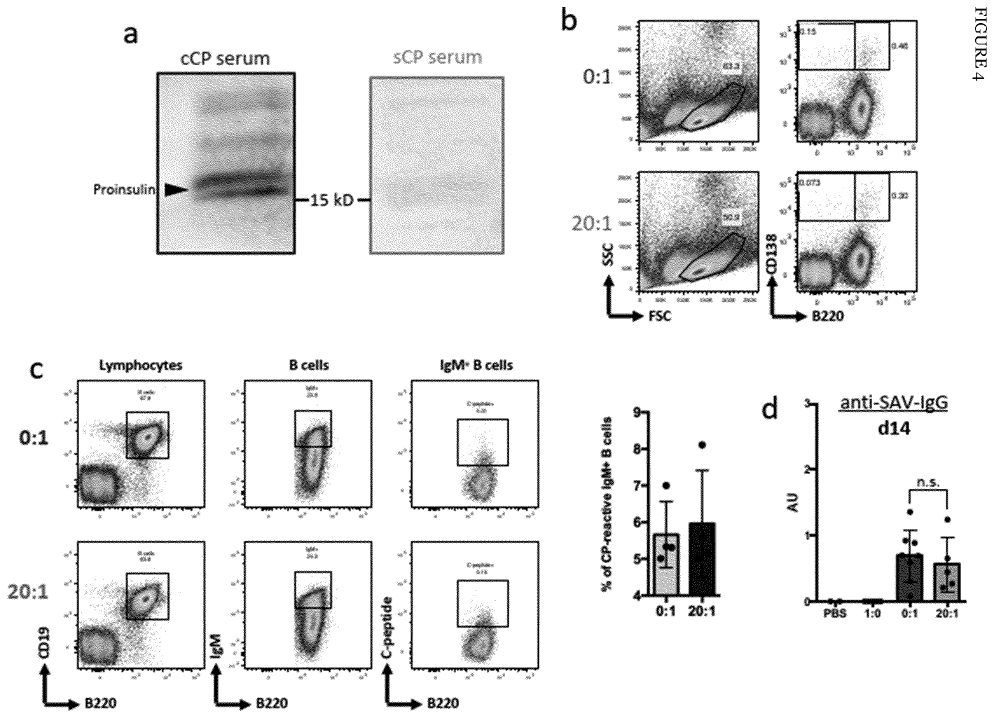

Presence of soluble peptides enhances IgM antibody responses: After testing hapten-specific antibody responses, it was tested whether the concept is valid for autoantigens and might thus provide a different scenario for the selection of B cells and the control of self-destructive immune responses. To avoid the usage of transgenic mice that artificially harbor mono-specific B cells expressing a defined BCR that recognizes either a transgene product or endogenous structure, insulin-associated autoantigens were selected as a physiologically relevant system for autoimmune diseases. During biosynthesis in the pancreas, proinsulin is cleaved into the well-known hormone insulin and the so-called C-peptide (CP) and both are secreted into the blood stream. While insulin is found in nanomolar amounts in the blood and plays pivotal role in the regulation of blood glucose levels and diabetes, C-peptide is barely detectable and is present at low picomolar quantities in the blood and seems to have no homeostatic function [30]. Using full length C-peptide or insulin-derived peptides, the autoreactive antibody responses towards an abundant and functionally important (insulin) should be investigated as compared to a barely detectable autoantigen without physiological function (C-peptide) (FIG. 3a). Moreover, in contrast to insulin C-peptide is not conserved (FIG. 3b).

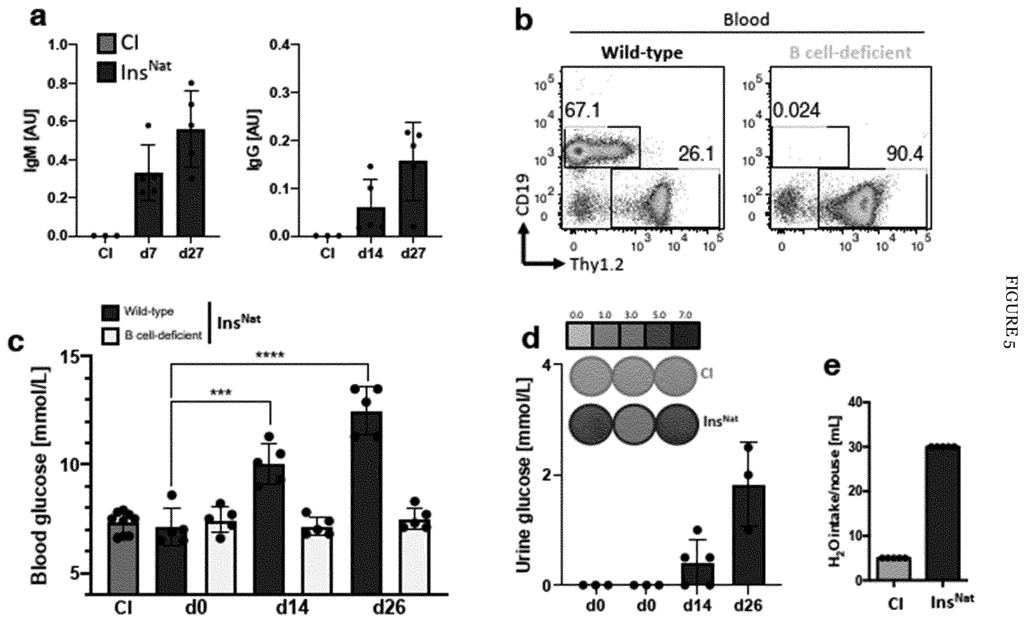

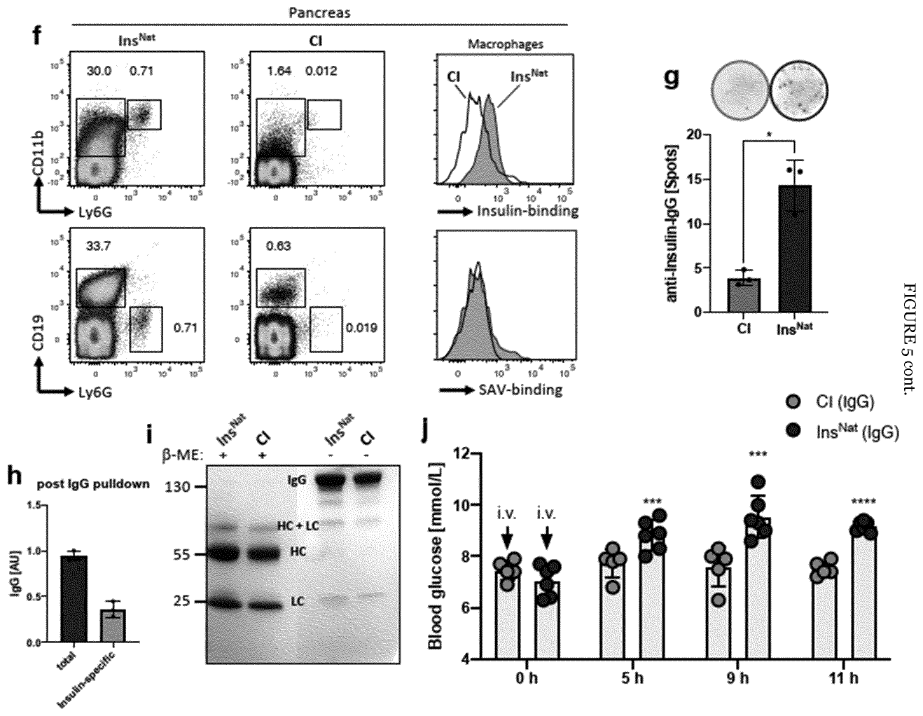

Either biotinylated C-peptides that were complexed by incubation were used with streptavidin (SAV). Alternatively, KLH was used as carrier coupled to the C-peptides to generate a multivalent complex antigen (cCP). The non-complexed form of the C-peptide (sCP) was used as soluble antigen. As with the NP hapten, wildtype mice were injected with sCP, cCP or combinations thereof to test their potential to induce autoreactive antibody responses (FIG. 3c). As expected, sCP induced no detectable IgM or IgG immune responses, while the multivalent form cCP induced both IgM and IgG as measured at d7 and 14, respectively (FIG. 3d). In addition to ELISA experiments, the serum from immunized mice was used to determine the specificity of the generated antibody responses. Western blot analysis using mouse serum revealed that mice immunized with cCP were positive for IgG antibodies recognizing pancreatic C-peptide (FIG. 4a). This is in full agreement with the hapten immunization and shows that soluble peptide, which is alone unable to induce a detectable immune response, prevents the production of IgG memory B cells. In fact, later challenge with the same antigen at d21 resulted in weak IgG response in mice immunized with sCP:cCP ratio of 20:1 as compared to mice immunized only with cCP, sCP:cCP ratio of 0:1 (FIG. 3d, d14 and d28 IgG). To confirm the memory response against C-peptide as autoantigen, a recall immunization at d42 was performed using cCP without the adjuvant CpG and detected a robust IgG response against C-peptide in the mice immunized only with sCP:cCP ratio of 0:1 (FIG. 3e).

In contrast to IgG, a C-peptide-specific IgM antibody response was induced upon recall immunization of sCP:cCP at 20:1 ratio (FIG. 3d, d28 IgM). FACS analysis of splenic B cells revealed no significant differences in the different groups of mice (Suppl. FIG. 3b, c). Moreover, no difference was detected in the IgG response against the carrier for the C-peptide (FIG. 4d).

These data suggest that soluble monovalent antigen modulates the immune response and determines the IgG:IgM ratio of antibody secreting cells during immune responses. This conclusion was confirmed by performing an ELISpot analysis to determine the number of IgG or IgM secreting cells in the different mouse groups. In full agreement with the serum Ig results, the ELISpot experiments showed that mice immunized with ratio 20:1 of sCP:cCP possess increased numbers of IgM secreting cells whilst the numbers of IgG secreting cells are decreased as compared to mice immunized with cCP, sCP:cCP ratio of 0:1 (FIG. 3f).

To test whether similar to NP immunization experiments, IgD is required for the regulation of B cell responsiveness by sCP:cCP ratios, the C-peptide immunization was performed in IgD knockout mice. The IgD knockout mice showed generally reduced IgG responses and no regulatory effect of the soluble peptide on the IgG antibody response observed in the mice immunized with sCP:cCP at 0:1 ratio (FIG. 3g).

Together, these data show that antibody responses can be directed against an autoantigen suggesting that the respective autoreactive B cells were neither clonally deleted by central tolerance nor functionally silenced by anergy. Most importantly, regardless of self or non-self-antigen, the results show that B cell responses are induced by multivalent antigen and modulated by soluble counterparts thereby regulating B cell responsiveness and the isotype of generated antibody. This results in a dynamic and pivotal B cell function that is completely different from the current view.

### Example 2: Autoantibody Responses Against Insulin

Multivalent native insulin induces harmful anti-insulin IgG responses: Since C-peptide can be hardly detected in the blood and has no known physiological relevance, it is not excluded that autoantibody responses might be feasible against autoantigens present at such extremely low concentrations. Therefore, the autoantibody responses against insulin were tested. First, the fundamental postulate was tested that autoreactive B cells are naturally present in the periphery and not deleted by central tolerance or turned unresponsive by anergy as proposed by the current view. According to this concept, the formation of autoantigen complexes triggers the secretion of autoreactive antibodies from naturally existing autoreactive peripheral B cells. To test this, autoantigen were generated complexes by incubating biotinylated native murine insulin with streptavidin (Ins^{Nat}). Importantly, the biotinylated murine insulin is biologically active as it regulates glucose metabolism similarly to its unbiotinylated endogenous counterpart when injected in soluble form (data not shown). Wild-type mice were injected with 10 μg of Ins^{Nat} complexes and monitored over time for the presence of anti-insulin antibodies in serum. In parallel, it was tested whether the immunized mice developed a diabetes-like dysregulation of glucose metabolism by monitoring glucose levels in blood and urine. Considerable amounts of anti-insulin IgM at day 7 were detected, while anti-insulin IgG was detected at d14 post injection of complexed insulin (FIG. 5a). Both isotypes were detected after boost immunization (d21) at d28. Importantly, the mice showed clear signs of diabetes as measured by increased concentrations of blood glucose starting by d7 (data no shown), continuing through d14 and further increasing after boost (d21) at d26 (FIG. 5c). To show that the elevated blood glucose levels depended on autoantibody production, 10 μg Ins^{Nat} complexes into B cell-deficient mice (mb-1 knockout mice lacking the BCR component Igα also known as CD79A) were injected and monitored blood glucose (FIG. 5b, c). Interestingly, no increase in blood glucose was observed in the B cell-deficient mice suggesting that the presence of B cells and autoantibody secretion are crucial for the development of diabetes symptoms observed in wild-type mice (FIG. 5c). Moreover, the increase in blood glucose was accompanied by detectable glucose in the urine of wildtype mice injected with complex Ins^{Nat} (FIG. 5d). In agreement with diabetes development, water consumption of wildtype mice injected with complex Ins^{Nat} dramatically increased (FIG. 5e). Due to the unexpected severity of diabetes symptoms the mice were sacrificed at day 27 and analyzed the pancreas and spleen.

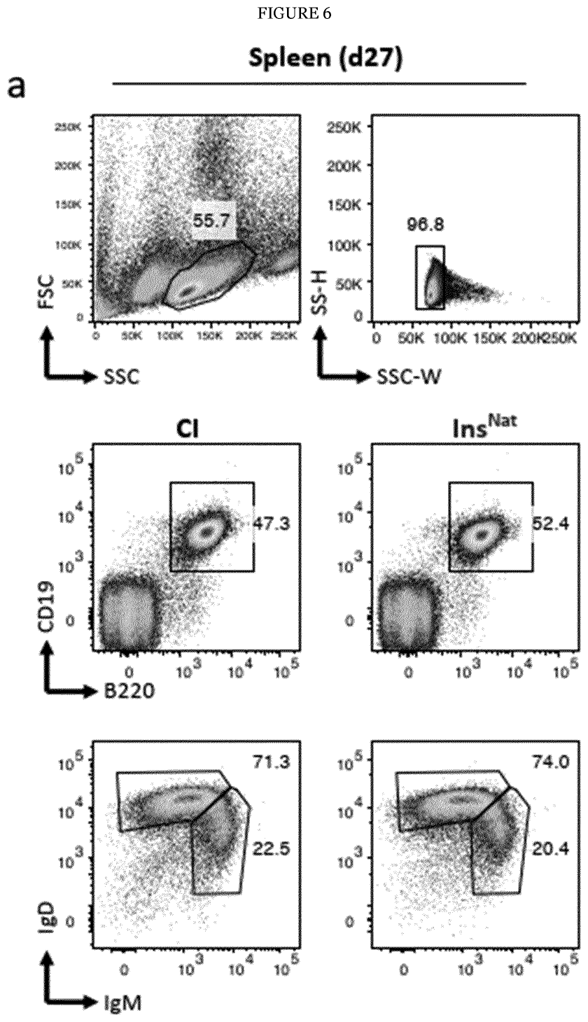

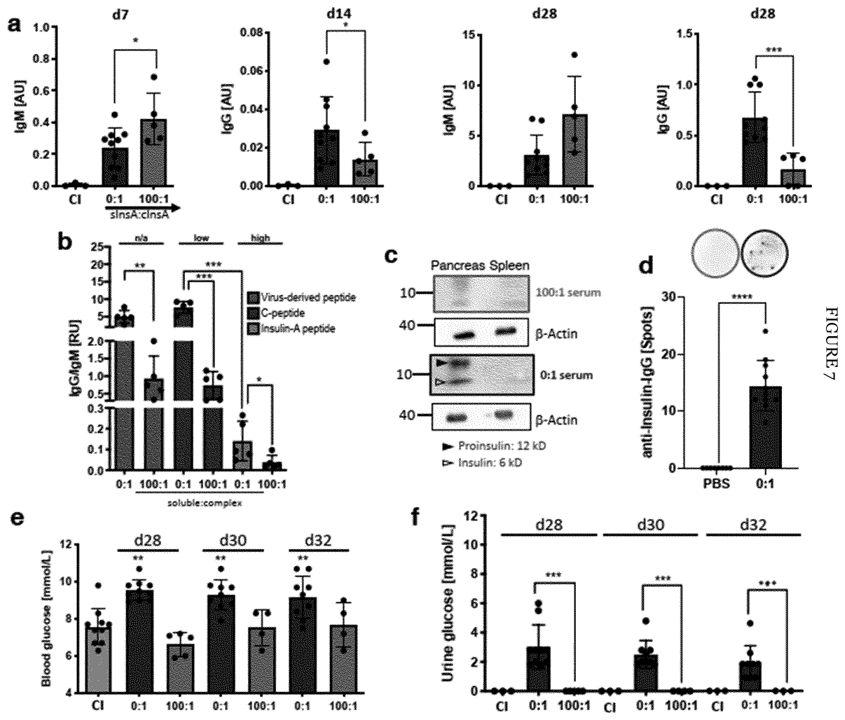

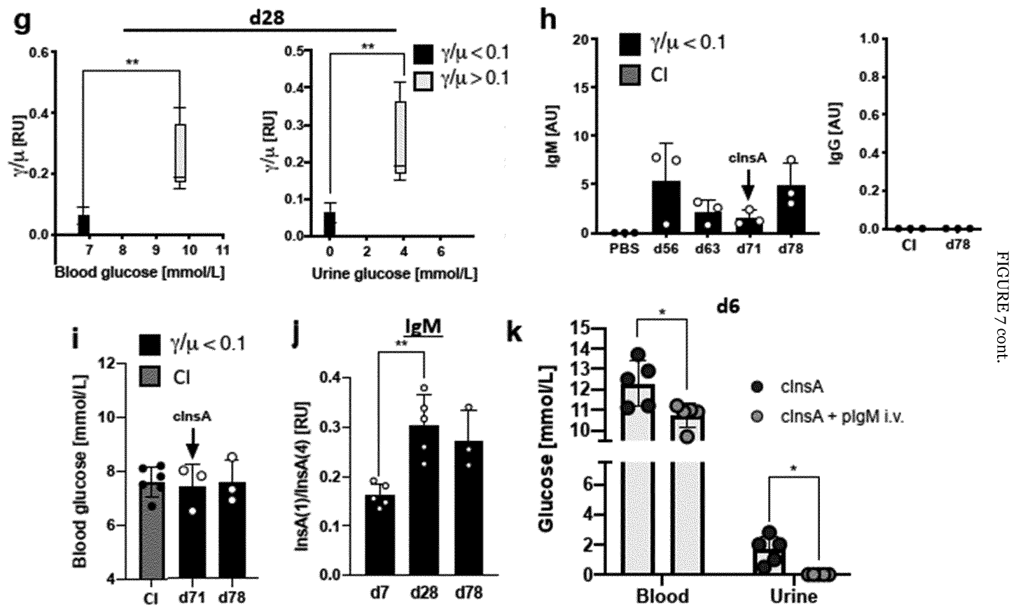

In contrast to control mice, complex Ins^{Nat} immunized mice showed highly increased recruitment of macrophages, neutrophils and B cells to the pancreas (FIG. 5f). Further, IgG+ macrophages of Ins^{Nat} complex immunized mice showed binding of native insulin (FIG. 5f. Thus, suggesting autoantibody-mediated acute inflammatory processes at the pancreas. While FACS analysis showed no difference of splenic B cells between control mice and those immunized with complex Ins^{Nat} (FIG. 6), however, ELISpot analysis revealed a significantly increased number of splenic B cells secreting anti-insulin IgG in mice injected with complex Ins^{Nat} (FIG. 5g).

To test whether the secreted IgG was responsible for the diabetes symptoms, IgG pulldown experiments using serum from mice injected with complex Ins^{Nat} and control immunization (FIG. 5h, i) were performed. Since the IgG purification is expected to result in dissociation of endogenous insulin from serum insulin-specific IgG (see methods section), we determined the anti-insulin IgG within total IgG after purification. It was found that up to 40% (0.4 mg/mg) of the IgG isolated from Ins^{Nat} mice was reactive to insulin suggesting that direct serum IgG measurements fail to detect the entire insulin-specific IgG due to binding to endogenous insulin (compare FIGS. 5a and 5h). To test the pathogenicity of isolated anti-insulin IgG, equal amounts of IgG from control immunization were intravenously injected or mice injected with complex insulin into wildtype animals and monitored blood glucose. It was found that injecting total IgG containing 2 μg anti-insulin IgG was sufficient to induce increased blood glucose in recipient mice suggesting that IgG from mice injected with complex insulin causes diabetes symptoms (FIG. 5j).

These data demonstrate that autoreactive B cells recognizing a pivotal metabolic hormone are neither deleted nor functionally silenced, but are present in the periphery and can induce severe autoimmunity when the balance of autoantigen is shifted towards multivalent forms.

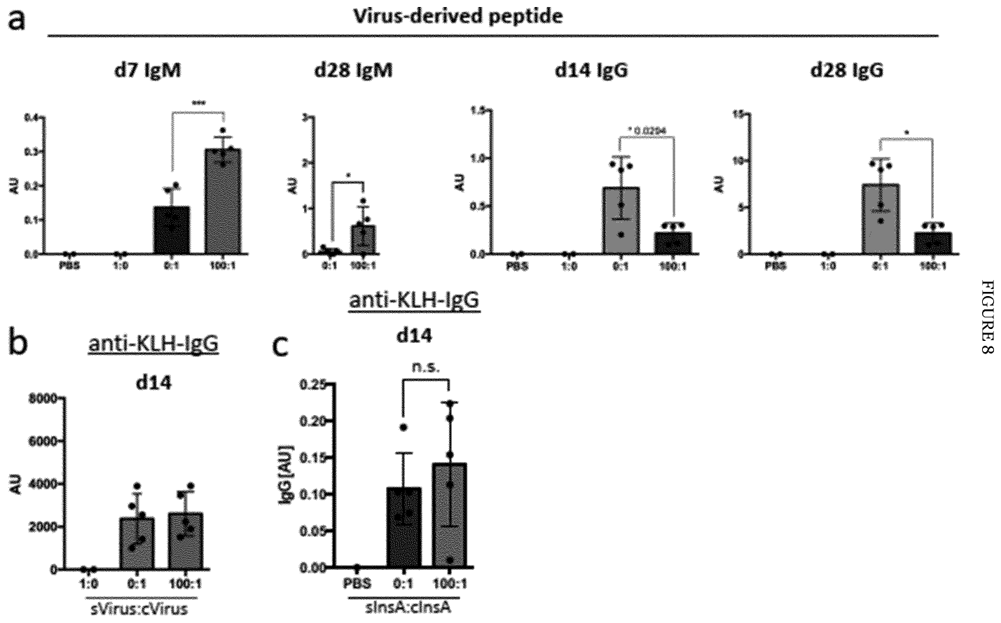

Insulin-derived epitope induces harmful anti-insulin IgG response: To further confirm the above findings, immunization experiments using an insulin-A chain-derived peptide sequence were performed, referred to as InsA (FIG. 3 b) which is a frequently reported epitope in autoantibody responses against insulin [32]. A virus-derived peptide from HIV gp12033 was included as a nonrelated foreign peptide (virus-peptide). As for C-peptide, the selected peptide was coupled to the carrier KLH to generate a complex polyvalent antigen (cInsA) which was then used in immunization experiments either alone or in combination with the soluble peptide (sInsA). Subsequently, the antibody responses against the immunogen was measured, InsA peptide, or native insulin to confirm the induction of harmful autoantibody responses. It was found that InsA induced IgM and IgG autoantibody responses recognizing native insulin (FIG. 7a). One week after boost (d21) at day 28, the multivalent insulin-derived peptide alone (sInsA:cInsA ratio of 0:1) readily induced the production of anti-insulin IgG, while addition of soluble peptide (sInsA:cInsA ratio of 100:1) resulted in profound reduction of this autoreactive IgG at day 28 (FIG. 7a). Importantly, the amount of autoreactive anti-insulin IgG is most likely higher than detected in direct serum ELISA as anti-insulin IgG bound to endogenous insulin escapes detection as described above (FIG. 5a, i).

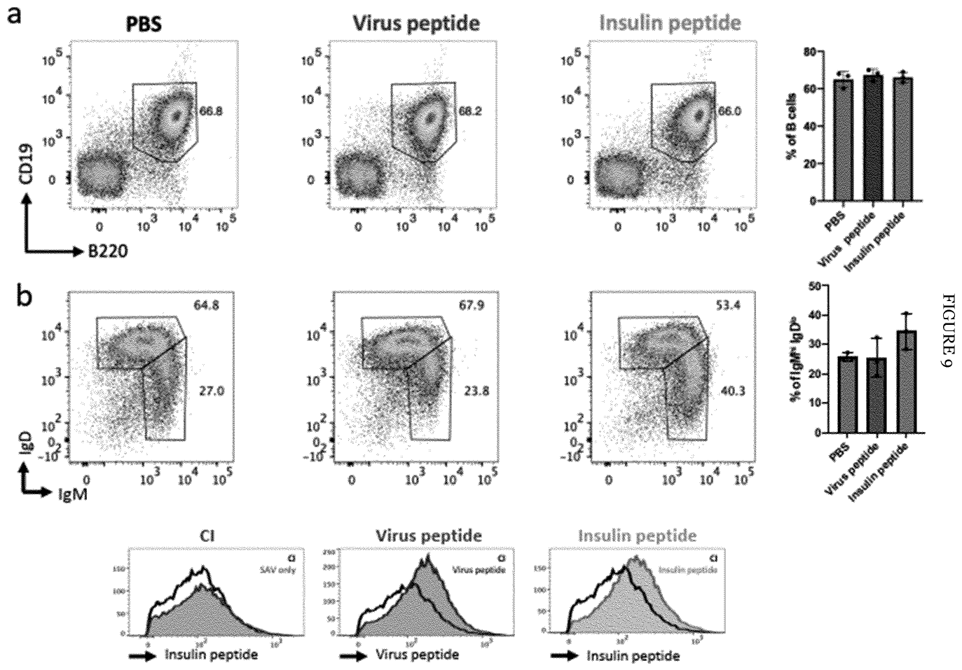

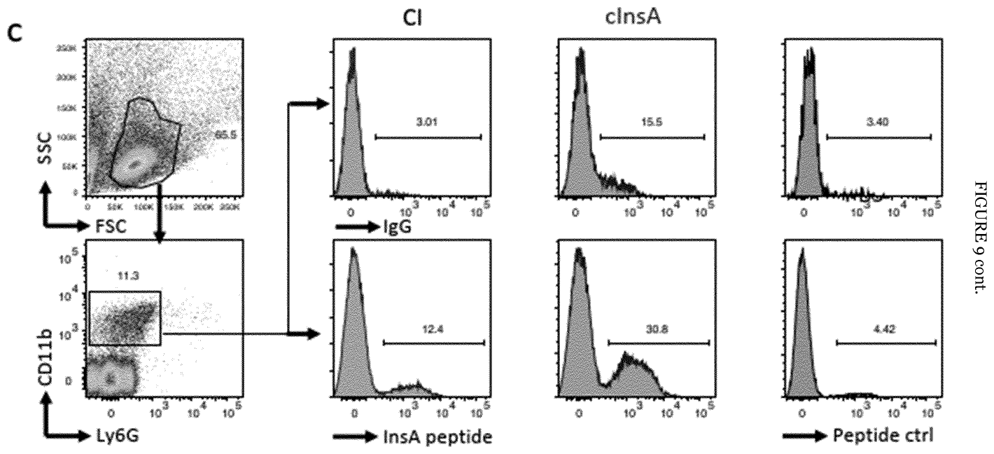

Notably, the presence of soluble InsA resulted in robust insulin-specific IgM production at d28, which was slightly reduced in the mice immunized with multivalent peptide alone (sInsA:cInsA ratio of 0:1) showing detectable anti-insulin IgM at d28 (FIG. 7a). This was not observed in mice immunized with the virus-peptide (FIG. 8a, b). In contrast to control peptides, insulin is present in relatively high amounts in the organism, suggesting that the presence of endogenous soluble insulin might modulate that immune response of the multivalent InsA thereby leading to increased autoreactive booster IgM responses. Taken together, the data indicate that the ratio of multivalent to monovalent antigen is mirrored by the ratio of antigen-specific IgG to IgM (γ/μ ratio) antibody responses at day 28 after booster immunization (FIG. 7b).

In contrast to the serum IgG of mice immunized in the presence of soluble peptide (sInsA:cInsA ratio of 100:1), serum IgG of mice immunized with multivalent peptide only (sInsA:cInsA ratio of 0:1) readily detected native insulin in western blot analysis (FIG. 7c). Moreover, ELISpot analysis using splenic B cells from mice immunized with cInsA confirmed the increased presence of autoreactive IgG secreting cells in respective mice (FIG. 7d).

To confirm that the increased anti-insulin IgG is associated with harmful autoimmune responses, it was tested whether mice immunized with cInsA (sInsA:cInsA ratio of 0:1) show signs of diabetes. It was found that about one week after booster immunization (d21) at day 28, this group of mice showed increased blood glucose and water intake by d27 to d33 (FIG. 7e & FIG. 10). In addition, it was tested whether the glucose concentration was also increased in the urine of mice immunized with multivalent insulin peptide (sInsA:cInsA, 0:1). In full agreement, the increased autoreactive anti-insulin IgG led to increased urine glucose concentrations (FIG. 7f). In contrast to autoreactive IgG, no detectable signs of autoimmune diabetes were observed in mice possessing increased amounts of autoreactive anti-insulin IgM in the booster immunization (FIGS. 7e & f).

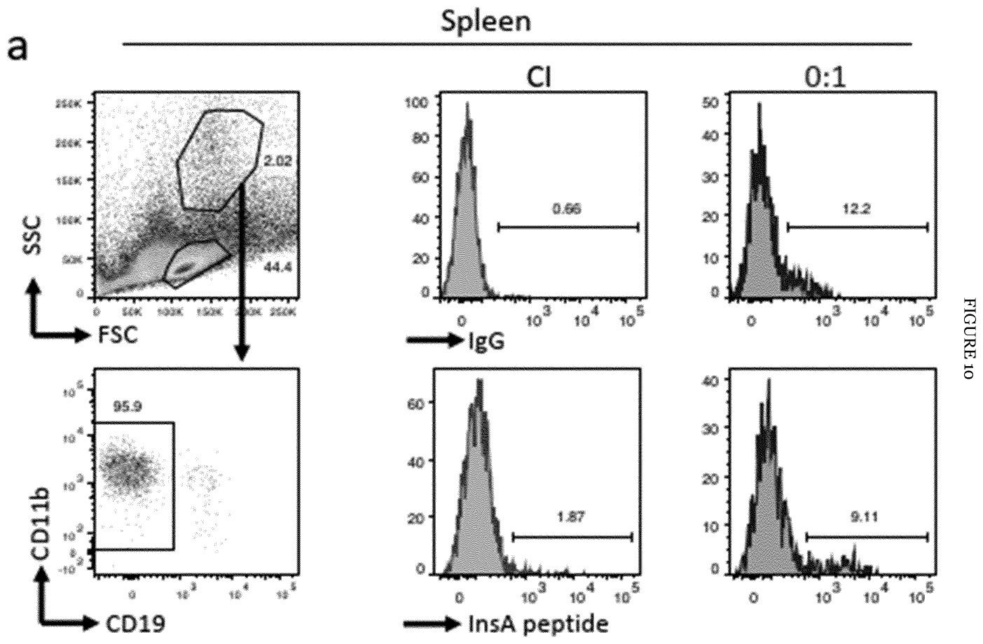

The presence of antigen-specific B cells at d28 after immunization was confirmed by FACS analysis (FIGS. 9a & b). Compared with controls, mice immunized with complex peptide only (sInsA:cInsA ratio 0:1) show increased proportion of macrophages in the pancreas which bound autoreactive IgG as determined by the increased InsA peptide binding (FIG. 9c). Similar results were observed in the spleen (FIG. 10).

Together, the data suggest that increased ratio of complex multivalent auto-antigen leads to increased amount of autoreactive IgG and subsequent self-destructive autoimmune responses in wild-type animals.

### Example 3: Protective Anti-Insulin-IgM Expression after InsA-Peptide Immunization

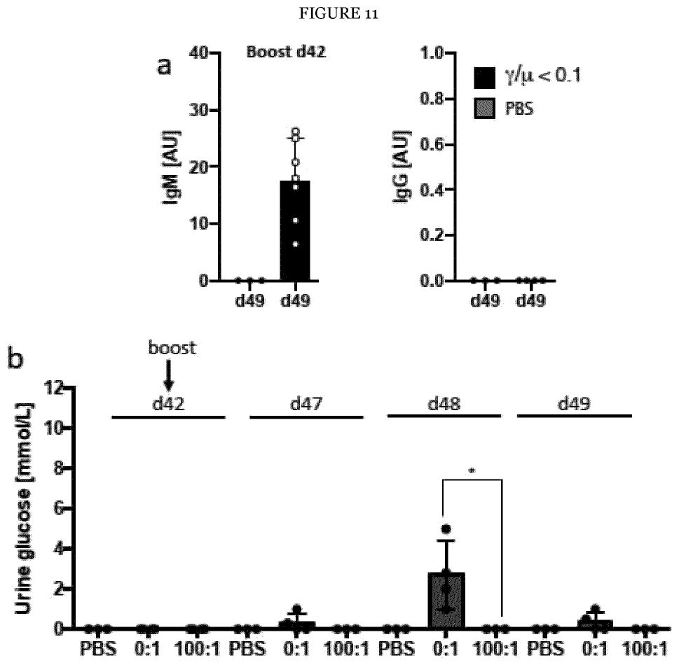

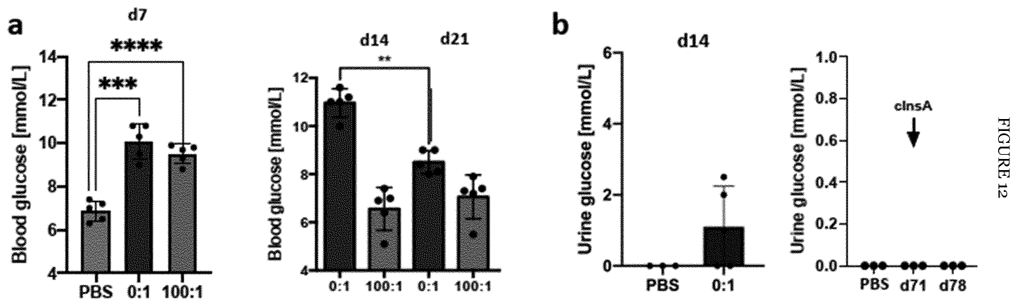

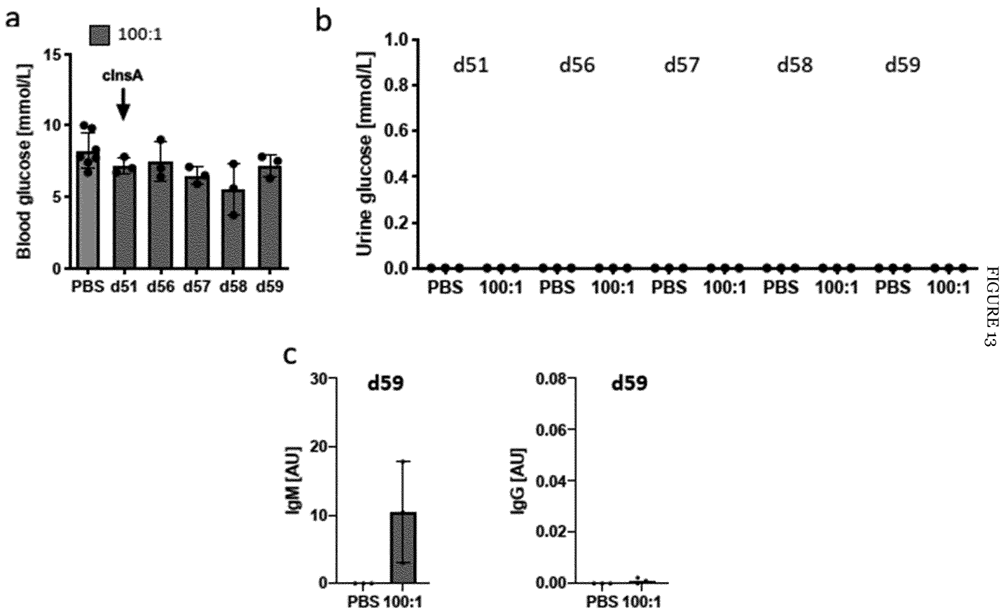

Monovalent autoantigen induces immune tolerance by protective IgM: Apart from the self-destructive role of autoreactive IgG, the data mentioned previously point towards a protective role of autoreactive IgM in diabetes. In fact, the results suggest that high anti-insulin IgM in comparison to corresponding anti-insulin IgG protects from deregulation of glucose metabolism and diabetes in the mice immunized with InsA (FIG. 7a-f). In full agreement, mice showing low ratio of insulin-reactive IgG to IgM (γ/μ<0.1) were protected from diabetes at d28 (FIG. 7g). A second InsA booster immunization at d42 resulted in anti-insulin IgM but no IgG when monovalent peptide was included (sInsA:cInsA ratio 100:1) and the corresponding mice showed no signs of diabetes between d42 and d49 (FIGS. 11a & b).

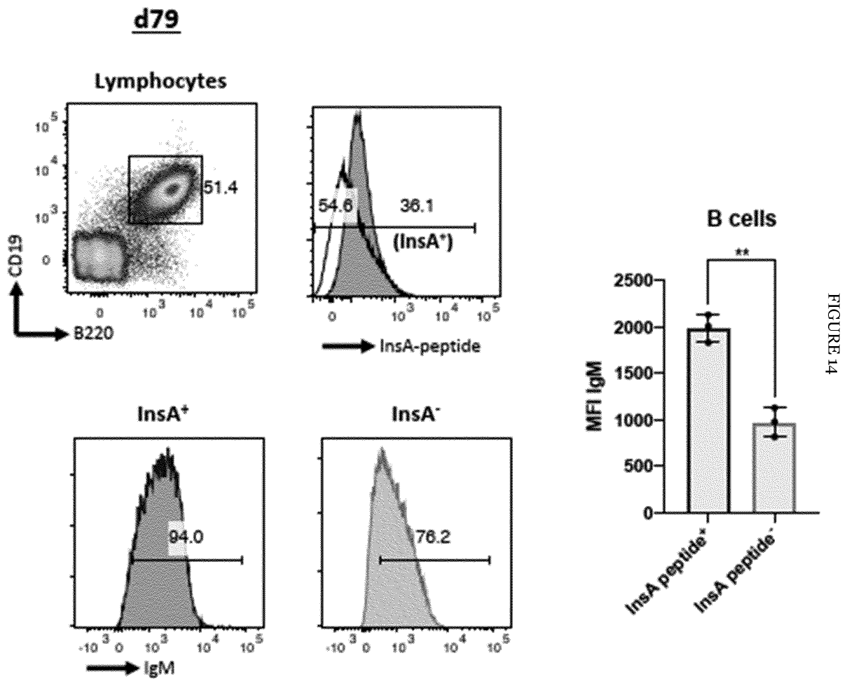

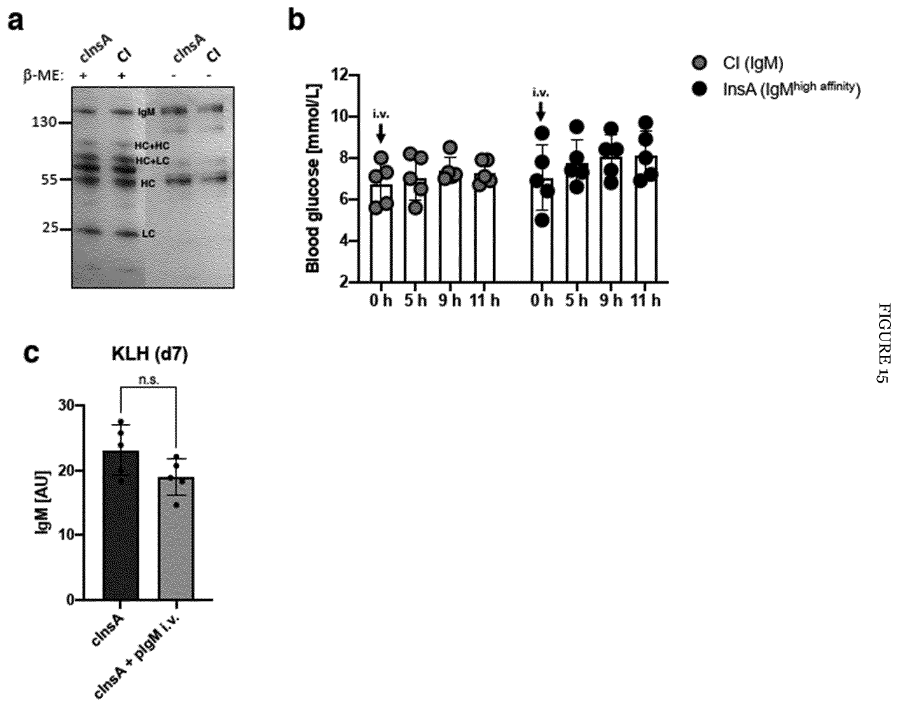

To directly test whether increased ratio of autoreactive anti-insulin IgM counters the negative effects on glucose metabolism induced by autoreactive anti-insulin IgG, the mice immunized initially in the presence of monovalent InsA peptide (sInsA:cInsA ratio 100:1) was challenged with only multivalent antigen (sInsA:cInsA, 0:1) at d51. Interestingly, the treatment that induced autoimmune diabetes from d14 to 28 (FIG. 12, d7 vs. d14), generated only autoreactive anti-insulin IgM response but neither anti-insulin IgG nor deregulation of glucose metabolism at d51 to 59 (FIG. 13 a-c).

These data suggest that primary immunization with the presence of monovalent InsA peptide (sInsA:cInsA ratio 100:1) induced tolerance against the pathogenic immunization with multivalent InsA (sInsA:cInsA ratio 0:1). Moreover, the findings indicate that this unique tolerance mechanism creates a novel class of memory responses by eliciting and maintaining the production of protective autoreactive IgM (pIgM). To further test this, the decline of the anti-insulin IgM concentration over time was monitored followed by anti-insulin recall responses (FIG. 7h). The inventors show that anti-insulin IgM persists for weeks and that booster cInsA immunization at day 71 induces only IgM, but no IgG without any signs of deregulated glucose metabolism (FIG. 7h, i & FIG. 14). Since the increase of antibody affinity towards antigen is usually associated with memory responses, ELISA experiments were performed to compare the affinity of the insulin-specific antibodies at different time points. It was found that IgM generated after booster InsA immunizations show higher anti-insulin affinity compared to the primary IgM collected at day 7 (FIG. 7j). Further, to examine the protective role of pIgM, mice were immunized with cInsA or cInsA together with intravenous injections of 50 μg purified IgM containing 5 μg of pIgM (FIG. 15a, b) every 48 hours starting from do. Interestingly, the presence of insulin-specific pIgM mitigated autoimmune dysglycemia and completely prevented glycosuria as observed in the mice immunized with cInsA only (FIG. 7k). To exclude that pIgM i.v. injections neutralized the immunogen (cInsA, i.p.), anti-carrier-ELISA was performed. As expected, no difference in anti-KLH-IgM levels were observed at day 7 (FIG. 15c).

Since insulin and the InsA peptide in particular are highly conserved between mouse and man (FIG. 3b), the data not only present a novel and dynamic concept for B cell tolerance, but also introduces a fundamental animal model for understanding autoimmune diabetes triggered by anti-insulin antibodies in humans.

### Example 4: Protective Memory Anti-Insulin-IgM is Monospecific

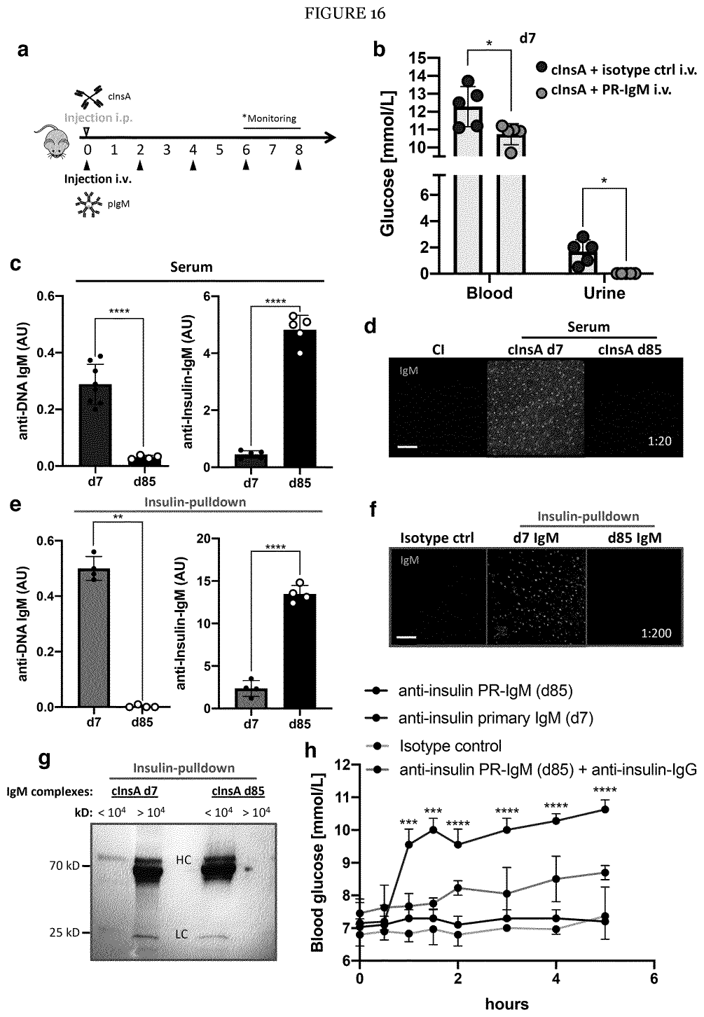

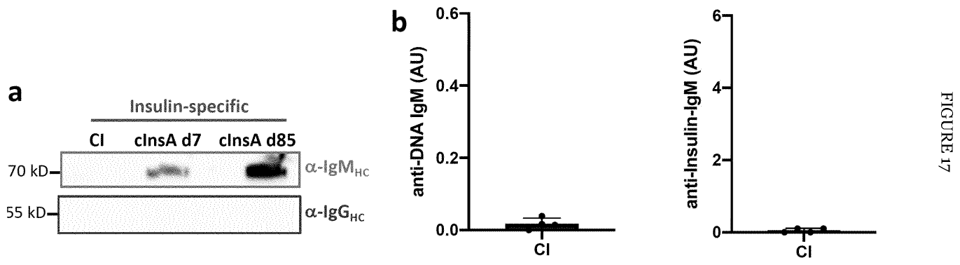

The results presented above point towards an unexpected fundamental difference between autoreactive primary IgM and PR-IgM. In fact, primary anti-insulin-IgM induced diabetes symptoms although produced at much lower quantity as compared to memory PR-IgM which possesses a higher insulin affinity but did not induce pathology. To directly test the protective function of autoreactive memory PR-IgM against destructive autoimmunity, mice were immunized with cInsA alone or cInsA together with intravenous injections of 50 μg total IgM containing 5 μg of anti-insulin memory PR-IgM every 48 hours starting from do (FIGS. 16a and b). Interestingly, the presence of insulin-specific PR-IgM mitigated autoimmune dysglycemia and completely prevented glycosuria on day 7 as compared to mice immunized with cInsA alone (FIG. 16b). To exclude that PR-IgM injections neutralized injected cInsA, we performed anti-carrier (KLH) ELISA and found no difference in anti-KLH-IgM levels between the two groups at day 7 (FIG. 15 C). These data suggest that memory anti-insulin PR-IgM prevents the depletion of insulin by primary anti-insulin IgM thereby preventing the initiation of diabetes. One explanation for the differences between the autoreactive primary and memory PR-IgM might be that primary IgM is polyreactive and might be produced by Bi B cells as a first line of immune protection. Presumably, this polyreactivity results in joint immune complexes with a high molecular weight containing multiple autoantigens allowing elimination by phagocytes thereby depleting the bound insulin. In contrast, autoreactive memory PR-IgM might be mono-specific for autoantigen and may therefore release the autoantigen after binding without formation of immune complexes. To test this, the polyreactive potential of primary IgM as compared to memory PR-IgM was analyzed. Anti-DNA ELISA (FIG. 16c) and indirect immune fluorescence using HEp-2 slides (FIG. 16d) showed that in contrast to primary IgM, memory PR-IgM is not polyreactive but specifically binds to insulin (FIGS. 16c and d).

To show that anti-insulin IgM is specifically responsible for the observed effects, the inventors performed insulin-specific pulldown assays using sera from InsA-immunized mice. The pulldown resulted in pure insulin-specific IgM as revealed by western blot analysis against insulin (FIG. 17). We performed anti-DNA ELISA (FIG. 16e) and indirect immune fluorescence on HEp-2 slides (FIG. 16f) using purified primary anti-insulin IgM or memory anti-insulin PR-IgM. The results confirm the finding that in contrast to primary IgM, purified anti-insulin PR-IgM is not polyreactive and specifically binds to insulin (FIGS. 16e and f). To directly test the hypothesis that primary anti-insulin IgM forms large immune complexes whereas PR-IgM does not, we incubated anti-insulin primary IgM or PR-IgM with insulin and DNA and determined the formation of immune complexes using size exclusion spin columns. In contrast to PR-IgM, we found that primary anti-insulin IgM forms mainly large complexes of >104 kD (FIG. 16g). To show that the purified primary anti-insulin IgM is responsible for the dysregulation of glucose metabolism, we intravenously injected 5 μg of purified anti-insulin primary IgM or PR-IgM and monitored blood glucose. In contrast to PR-IgM, we observed a vigorous increase in blood glucose after injection of purified primary anti-insulin IgM (FIG. 16h). Interestingly, the increase in blood glucose emerged faster after injection of purified anti-insulin primary IgM as compared to total primary IgM (FIG. 16h).

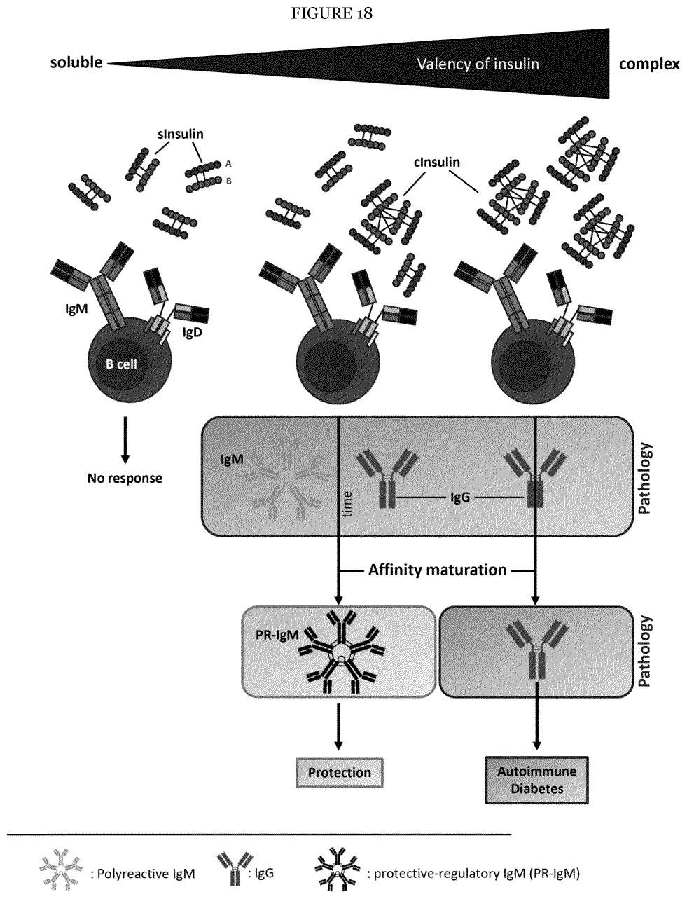

In summary, these data suggest that increased specificity to autoantigen is important for autoreactive memory PR-IgM to be protective during immune responses (FIG. 18). Moreover, the induced generation of autoreactive PR-IgM is most likely a critical step in B cell tolerance.

### Example 5: Immunization Scheme

The impact of the immunization concept of the invention with regard to vaccine design was tested using pathogen-specific antigens derived from SARS-CoV-2 coronavirus causing Covid-19. During infection, SARS-CoV-2 coronavirus binds via the receptor-binding domain (RBD) to angiotensin-converting enzyme 2 (ACE2) on the host cell surface. Thus, triggering antibody responses blocking the RBD/ACE2 interaction is considered to be key for preventing coronavirus infection. Thus, the inventors used RBD from SARS-CoV-2 to the role of antigen form in immune responses during immunization.

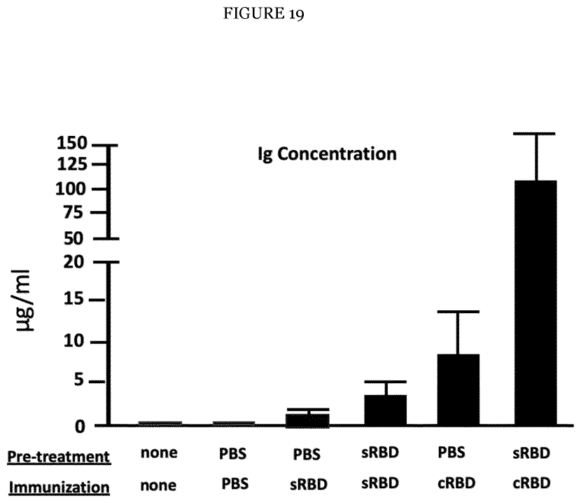

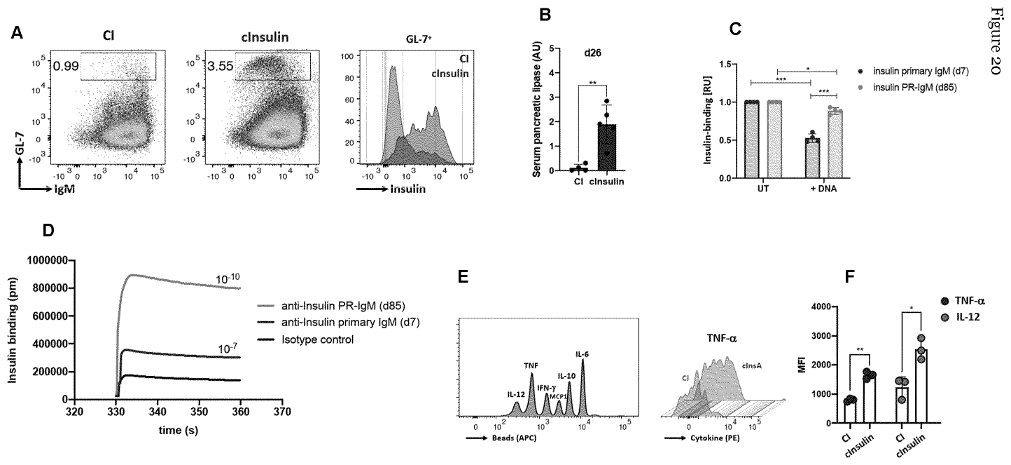

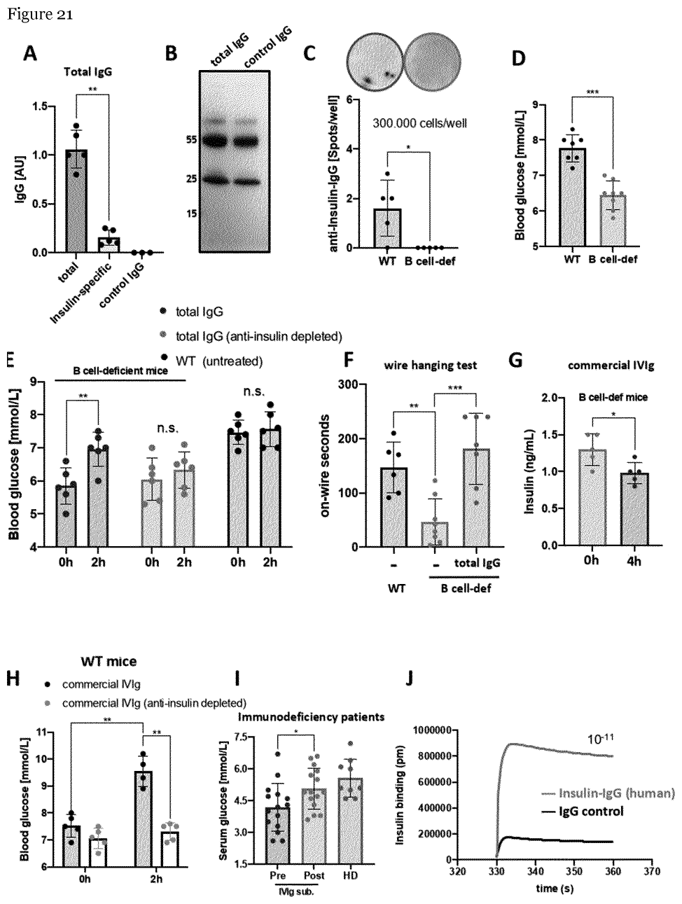

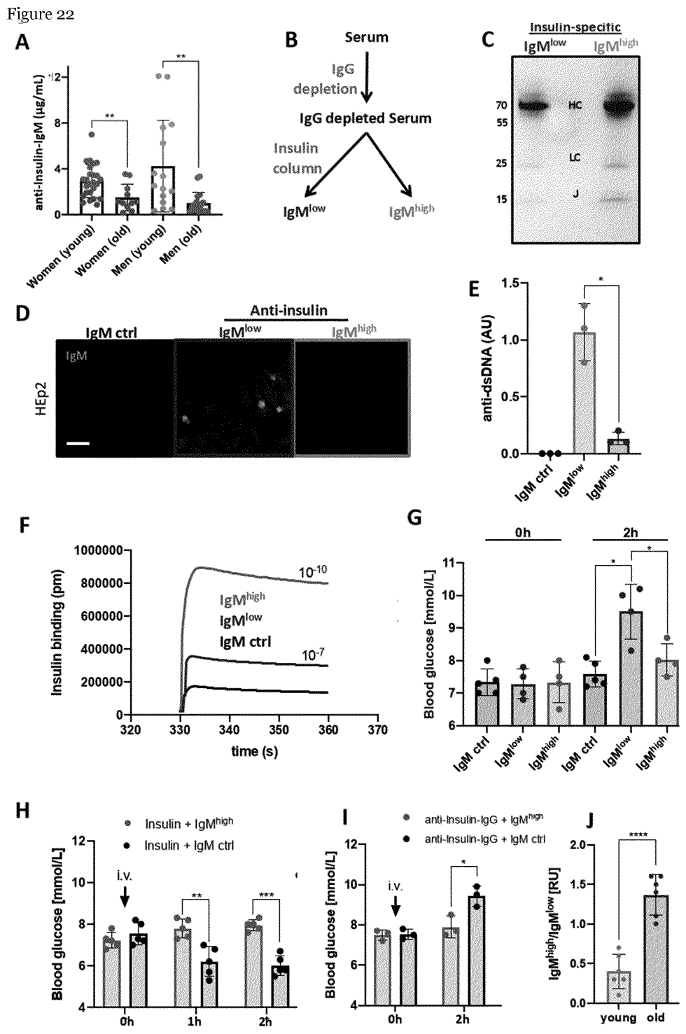

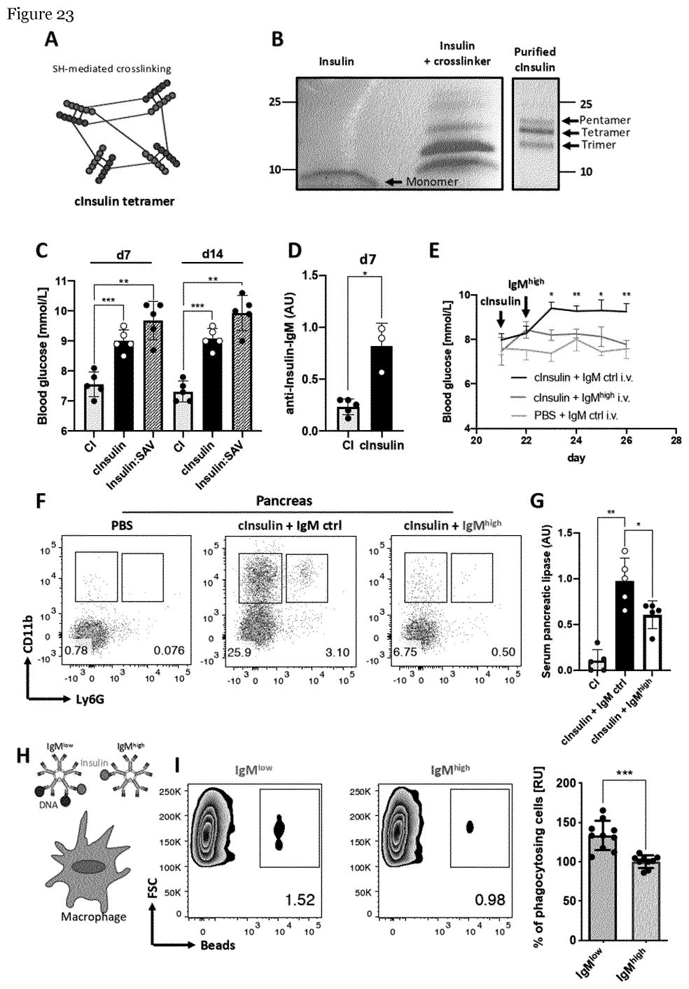

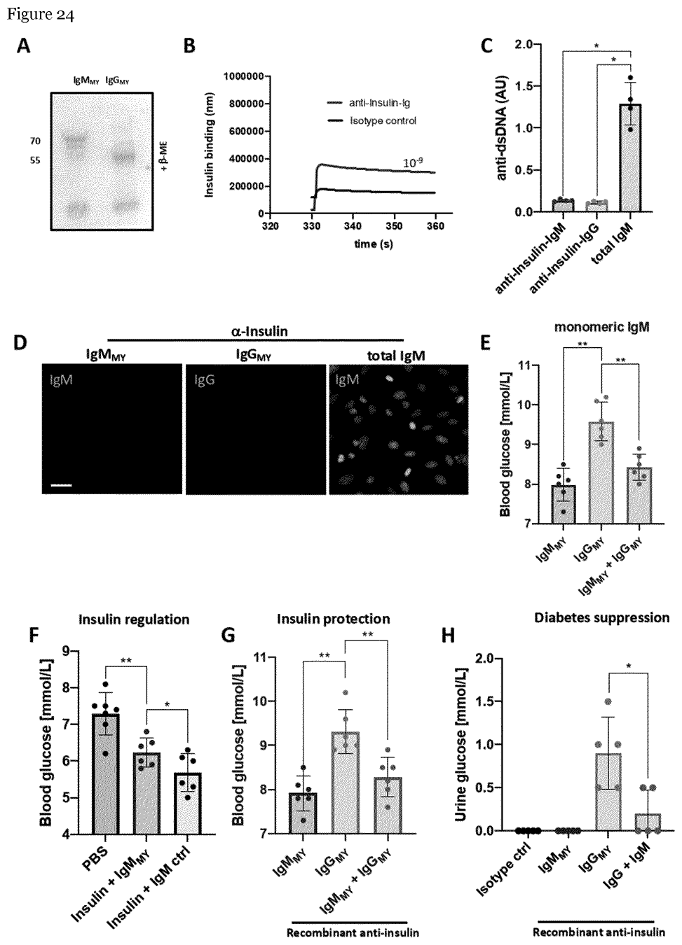

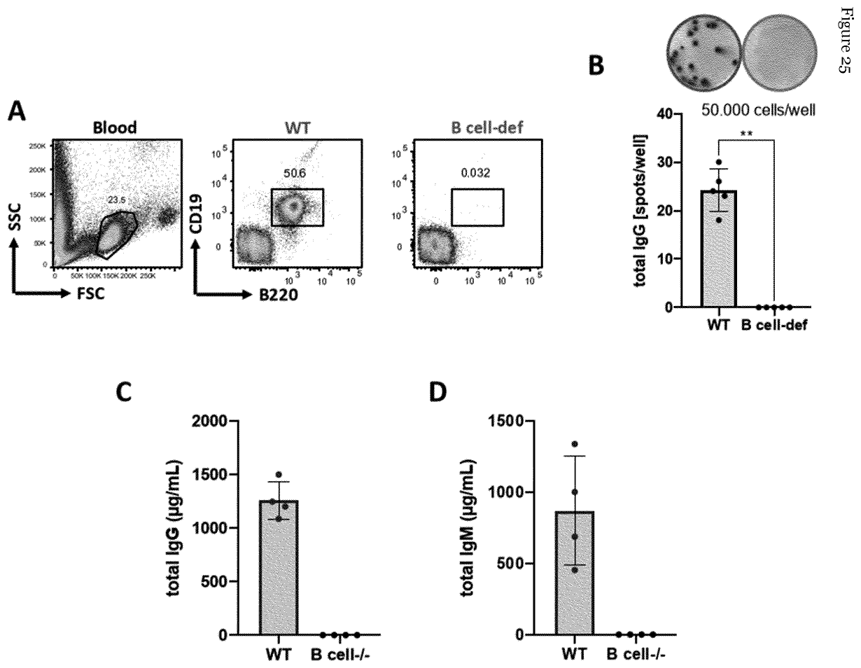

It was found that immunization with complex RBD (cRBD) (For complexation with streptavidin and biotinylated RBD were used at a ratio of 4:1) induces a stronger IgG immune response as compared with soluble RBD (sRBD). For production of RBD, an expression vector encoding hexahistidine-tagged version of RBD was transiently transfected into HEK293-6E cells (Amanat, F., et al., 2020, Nature medicine, 26(7), 1033-1036). Soluble RBD was purified from the supernatant 5 days after transfection by nickel-based immobilized metal affinity chromatography (TaKaRa)). However, the antibody concentration was not sufficient to allow virus neutralization using in-vitro infection experiments. Hence, it was tested whether pretreating the mice with sRBD prior to immunization boosts immune responses. In fact, pre-treatment of the mice with soluble RBD two weeks prior to immunizations resulted in greatly augmented immune response (FIG. 19). Importantly, the serum of the pretreated mice showed an enormously high capacity to prevent SARS-CoV-2 infection in vitro.

Moreover, it was found that different ratios of sRBD to cRBD in the composition of the immunization cocktail result in different ratios of immunoglobulin isotypes (i.e. IgG to IgM) which allow refined control of immune responses after immunization.

### Example 6: Anti-Insulin IgG Regulates Blood Glucose Concentration

We noticed that a considerable amount of total IgG isolated from wildtype (WT) mice was reactive to insulin (FIGS. 21A & 21B). To confirm these data, we performed ELISpot assays and found that anti-insulin IgG secreting B cells are present in the spleen of WT mice (FIG. 21C). When we measured the blood glucose concentrations in WT and B cell-deficient mice, which cannot produce antibodies, we detected a surprising difference. Unexpectedly, the B cell-deficient mice showed abnormally reduced blood glucose levels as compared to WT controls (FIG. 21D).

To test whether this abnormal decrease is caused by antibody deficiency, we injected total IgG from WT mice, or an anti-insulin IgG depleted control of the same total IgG, intravenously into B cell-deficient mice. We found that blood glucose concentration increased with the total murine IgG, but not with the anti-insulin IgG depleted control (FIG. 21E). In order to test the consequence of reduced steady-state blood glucose on the fitness, we performed wire hanging tests to assess motor function and found that B cell deficient mice have significantly reduced wire hanging times as compared to WT controls. Importantly, this deficit in wire hang times was restored after intravenous injection of total murine IgG (FIG. 21F). In addition, B cell-deficient mice also showed dysregulated blood glucose levels after rotarod exercise.

Since total IgG preparations from healthy donors are often used as intravenous immunoglobulin (IVIg) injection in the treatment of immunodeficiency we tested the presence of anti-insulin IgG in these preparations. All preparations contained substantial amounts of anti-insulin IgG. However, the anti-insulin IgG concentration seemed to be increased if the USA was the country of origin. Since insulin is highly conserved between man and mouse, we injected human IVIg into the B cell deficient mice and detected a decrease in insulin concentration (FIG. 21G). Moreover, injecting 50 μg of human IVIg into WT mice led to increased blood glucose and this effect required anti-insulin IgG because depletion of the anti-insulin IgG from human IVIg prevented the IVIg-induced increase in blood glucose (FIG. 21H).

To test whether the IVIg injection shows similar results in human patients suffering from antibody deficiency, we monitored blood glucose before and after IVIg injection. Similar to B cell deficient mice, antibody deficient patients showed reduced blood glucose concentrations as compared to healthy donors. Importantly, the concentration of blood glucose increased and reached normal levels after IVIg injection (FIG. 21I). Further, immunodeficiency patients that received IVIg showed decreased serum insulin levels.

To show that the anti-insulin IgG present in IVIg is specific for insulin, we determined the affinity via bio-layer interferometry (BLI). A dissociation constant of 10-11 suggests that the anti-IgG is highly specific for insulin (FIG. 21J).

These data suggest that anti-insulin IgG is present in healthy individuals and might be required for the regulation of blood glucose concentration.

### Example 7: Regulation of Blood Glucose by Anti-Insulin IgM

To further confirm our finding about the presence of anti-insulin antibodies in healthy individuals, we assessed the anti-insulin IgG and IgM in the blood of different age groups. We found that anti-insulin IgG was similar in young and aged humans, while anti-insulin IgM seemed to decline with age in males and females (FIG. 22A). Interestingly, the human anti-insulin IgM recognizes multiple epitopes on insulin.

In agreement with the high specificity, the anti-insulin IgG showed no binding to any cellular structure in indirect immunofluorescence assay (IIFA) on HEp-2 cells, which is a commonly used method for detection of anti-nuclear antibodies. The anti-insulin IgM however, consisted of two fractions that can be biochemically separated according to their affinity to insulin. Low-affinity anti-insulin IgM is eluted from the insulin column at higher pH (5) as compared to high-affinity anti-insulin IgM which requires acidic conditions (pH=2.8) for elution (FIG. 22B, 22C). The low affinity IgM shows polyreactivity as detected by binding to nuclear structures in IIFA and dsDNA binding in ELISA, whereas the high affinity IgM is virtually negative in these assays (FIG. 22D, 22E). Furthermore, we confirmed the difference in affinity by performing BLI assays and found that high affinity and low affinity IgM to possess a dissociation constant of 10⁻¹⁰ and 10⁻⁷, respectively (FIG. 22F). To test the effect of the different IgM fractions on glucose metabolism, we injected identical amounts of insulin-reactive IgMhigh and IgMlow into WT mice. Increased blood glucose was observed within two hours after injection in the mice that received IgMlow, whereas IgMhigh did not significantly alter blood glucose levels (FIG. 22G). Moreover, we tested whether IgMhigh plays a regulatory role under conditions of abnormally increased insulin concentrations that may cause hypoglycemia. To this end, we injected 0.1 μg insulin in combination with IgMhigh or unspecific IgM isotype control. Strikingly, the presence of anti-insulin IgMhigh, but not the IgM isotype control, prevented the drastic decrease in blood glucose that occurred immediately after insulin injection (FIG. 22H). To further test the regulatory role of IgMhigh in protecting insulin from IgG-mediated degradation, we combined the anti-insulin IgMhigh with anti-insulin IgG purified from IVIg preparations. The data show that the anti-insulin IgMhigh acts as PR-IgM as prevents the IgG-mediated neutralization of insulin which results in increased blood glucose levels (FIG. 22I). These data suggest that anti-insulin IgMhigh is important for regulating glucose metabolism by protecting insulin from IgG-mediated neutralization and by binding excessive insulin thereby preventing drastic declines in insulin concentrations. The decrease in insulin-reactive IgM with age (FIG. 21A) prompted us to test whether the anti-insulin IgMhigh or IgMlow is affected by this decrease. We determined the amount of anti-insulin IgMhigh or IgMlow in young and old healthy donors and found that the ratio of anti-insulin IgMhigh increases with age (FIG. 22J).

Together, these data suggest that glucose metabolism is regulated by different classes of antibodies and that anti-insulin IgMhigh acts as PR-IgM that regulates glucose metabolism by regulating insulin homeostasis which seems to be particularly important with age.

### Example 8: Induction of Anti-Insulin Antibodies by Insulin Complexes

To investigate whether complexed autoantigen is capable of inducing autoreactive antibody responses independent of any adjuvants, we incubated insulin with a typical homobifunctional crosslinker, 1,2-Phenylene-bis-maleimide, which covalently binds to free sulfhydryl groups in proteins thereby crosslinking the protein of interest (FIG. 23A). Importantly, sulfhydryl group-containing drugs were reported to induce anti-insulin autoantibodies. Moreover, increased pancreas activity and elevated insulin production result in abnormal formation of disulfide bonds between the insulin peptides which may generate abnormal insulin forms that are more susceptible for sulfhydryl group-mediated crosslinking, and thus complex formation, under conditions of oxidative stress. The homobifunctional crosslinking of insulin with 1,2-Phenylene-bis-maleimide was tested in SDS page and the crosslinked insulin was purified using size exclusion spin columns excluding monomeric and dimeric insulin (FIG. 23B). The insulin complexes were dialyzed and injected into WT mice, 5 μg per mouse, without any additional adjuvants. As control, we performed a typical immunization using CpG as adjuvants and streptavidin as a foreign carrier. We found that the insulin complexes lead to increased blood glucose and anti-insulin IgM at d7 of treatment similar to the immunization (FIG. 23C, 23D). In addition, insulin-reactive IgG was detectable by ELISA on d14 and d26. Repeated injection of insulin complexes at d21 resulted in further deregulation of glucose metabolism (FIG. 23E). Thus, we injected anti-insulin IgMhigh at d22, one day after injection of the insulin complexes. We found that anti-insulin IgMhigh was able to prevent the blood glucose deregulation induced by the injection of insulin complexes (FIG. 23E).

Further, we found that anti-insulin IgMhigh prevents pancreas inflammation and damage as shown by the decrease of macrophage (CD11b+/LY6G+) and neutrophil (LY6G+) infiltration in the pancreas and the decrease of serum pancreatic lipase in blood (FIG. 23F, 23G).

As a mechanism for the protective role of anti-insulin IgMhigh as compared to anti-insulin IgMlow we proposed that the polyreactivity of the latter, which also binds dsDNA, induces the formation of immune complexes that can be phagocytosed by macrophages, while anti-insulin IgMhigh is highly specific for insulin and thus do not form large immune complexes that are easily phagocytosed by macrophages. To test this, we incubated anti-insulin IgMhigh or anti-insulin IgMlow with insulin in the presence of genomic dsDNA, (FIG. 23H). We found an increased binding/phagocytosis of anti-insulin IgMlow as compared with anti-insulin IgMhigh (FIG. 23). In addition, IgMhigh was able to protect insulin from degradation, as the decline of insulin was greater in the supernatants containing anti-insulin IgMlow as compared with anti-insulin IgMhigh antibodies.

These data show that anti-insulin antibodies can be generated under conditions activating the formation of insulin complexes, which results in deregulated glucose metabolism that can be counteracted by anti-insulin IgMhigh that acts as PR-IgM.

### Example 9 Recombinant Anti-Insulin IgM is Able to Regulate Blood Glucose

The above results suggest that insulin-specific PR-IgM might be of great therapeutic interest, as it regulates insulin homeostasis and might prevent pancreas malfunction, both of which essential for normal physiology and prevention of diabetes. According to our data, an anti-insulin IgM can act as PR-IgM if it possesses high affinity to insulin and is not reactive to autoantigens such as dsDNA or nuclear structure in IIFA. We hypothesized that a human insulin-specific IgG antibody can be converted into insulin-specific PR-IgM by exchanging the constant region.

Hence, we cloned and expressed a published human insulin-specific antibody [60] as IgG1 (anti-insulin IgGrec) and IgM (anti-insulin IgMrec) (FIG. 24A). To test the quality of our in vitro produced antibodies, we assessed their glycosylation by PNGaseF treatment, which resulted in reduced molecular weight as compared to untreated controls suggesting a functional glycosylation. We determined the affinity of both IgG and IgM to be 10⁻⁹ (FIG. 24B). Almost no dsDNA binding was observed in ELISA and no nuclear staining was observed in IIFA as compared to total human serum IgM (FIG. 24C, 24D). Moreover, we tested if the monomeric anti-Insulin-IgM is capable of protecting insulin from degradation. Anti-Insulin IgG led to blood glucose increase which was abolished when monomeric anti-Insulin IgM was present (FIG. 24E).

To test whether the resulting recombinant human anti-insulin IgMrec possesses protective regulatory functions, we co-injected it with insulin and found that anti-insulin IgMrec prevents a drastic drop in glucose concentration induced by excess of insulin (FIG. 24F). Moreover, anti-insulin IgMrec protects insulin from anti-insulin IgGrec mediated neutralization, as it prevents the increase in blood glucose induced by anti-insulin IgGrec (FIG. 24G). In addition, anti-insulin IgMrec counteracts the leak of glucose into urine (FIG. 24H).

These data suggest that expressing a high affinity insulin-specific antibody as IgM regulates insulin homeostasis, prevents a deregulation of blood glucose concentration and grants novel strategies for treatment of insulin-associated disease and disorders.

### Example 10: Prediction of Disease Parameters

The highly autoreactive primary IgM repertoire represents a high risk for autoreactive damage if high affinity PR-IgM cannot be generated by secondary immune responses and somatic hypermutation. Therefore the memory IgM repertoire consists mostly of PR-IgM generated in the course of adaptive tolerance. Somatic hypermutation leads to failure in PR-IgM generation and autoimmune damage induced by the primary IgM. Furthermore all forms of hyper IgM syndrome (HIGM) are associated with severe autoimmunity. HIGM patients are particularly prone to developing IgM-mediated autoimmune diseases such as immune thrombocytopenia, hemolytic anemia and nephritis. We measure the affinity of autoreactive IgM antibodies that cause autoimmune and/or insulin related diseases and consider (i) low-affinity to autoantigens as a risk factor for disease development, disease progression and/or mortality and (ii) and high-affinity autoreactive IgM as protective.

### Materials and Methods

### Mice Used for Example 1-5

8-30-week-old C57BL/6 mice and B cell-deficient mice were immunized intraperitoneally (i.p.) with a mixture of 13-50 μg antigen with 50 μg CpG-ODN1826 (Biomers) in 1×PBS. Control immunization (CI) mice received PBS and CpG-ODN1826 (50 μg/mouse). Native biotinylated murine insulin was purchased from BioEagle.

### Mice Used for Example 6-9

8-15-week-old female C57BL/6 mice and mbi mice 45 were intraperitoneally (i.p.) injected with a mixture of 10 μg antigen (cInsulin or Insulin-bio:SAV) in 1×PBS. Control injections (CI) mice received PBS in a total volume of 100 μL/mouse. Animal experiments were performed in compliance with license 1484 for animal testing at the responsible regional board Tübingen, Germany. All mice used in this study were either bred and housed within the animal facility of the Universiry of Ulm under specific-pathogen-free conditions, or obtained from Jackson company at 6 weeks of age. All animal experiments were done in compliance with the guidelines of the German law and were approved by the Animal Care and Committees of Ulm University and the local government.

### Peptides

C-Peptide peptides (RoyoBiotech, Shanghai), Insulin and virus-derived peptides (SEQ ID NO: 43; SEQ ID NO: 44) (Peptides&Elephants, Berlin) were dissolved according to their water solubility in pure water, 1% DMSO or 1% Dimethylformamide (DMF). The virus-derived peptides (SEQ ID NO: 43; SEQ ID NO: 44) were coupled to Biotin or KLH, respectively. An amount of 1 mg was purchased and dissolved in a volume of 1 ml. 10 to 50 μg of KLH-coupled peptide were used for immunization of mice via intraperitoneal injection. For covalent coupling of peptides to key hole limpet hemocyanin (KLH) a N-terminal cysteine was added. Coupling of peptides to Streptavidin (SAV, ThermoScientific) was done by addition of biotin to the N-terminus. The C-terminus was left with an OH-group for better handling.

### Crosslinking of Native Insulin and InsA Peptides

Native human insulin (Merck) was pre-diluted in PBS to 1 mg/mL. Chemical thiol-crosslinking was done using 1,2-Phenylen-bis-maleimide (Santa Cruz, 13118-04-2) at 10 μg/mL and afterwards removed by using a 10 kD cut-off spin column (Abcam, ab93349). Purified insulin complexes (cInsulin) were used for intraperitoneal injections at 10 μg per mouse in 100 μL total volume.

### Flow Cytometry

Cell suspension were Fc-receptor blocked with polyclonal rat IgG-UNLB (2,4G2; BD) and stained according to standard protocols. Biotin-conjugated peptides/antibodies were detected using Streptavidin Qdot605 (Molecular Probes; Invitrogen). Viable cells were distinguished from dead cells by usage of Fixable Viability Dye eFluor780 (eBioscienc). Cells were acquired at a Cato II Flow Cytometer (BD). If not stated otherwise numbers in the plots indicate percentages in the respective gates whilst numbers in histogram plots state the mean fluorescence intensity (MFI).

### Enzyme-Linked Immunosorbent Assay (ELISA)

96-Well plates (Nunc, Maxisorp) were coated either with, native Insulin (Sigma-Aldrich, Cat. 91077C), Streptavidin (ThermoScientific, Cat. 21125), or calf thymus DNA (ThermoScientific, Cat. 15633019), with 10 μg/mL, or anti-IgM, anti-IgG-antibodies (SouthernBiotech). Loading with a biotinylated peptide (2.5 μg/mL) of SAV-plates and blocking was done in 1% BSA blocking buffer (Thermo Fisher). Serial dilutions of 1:3 IgM or IgG antibodies (SouthernBiotech) were used as standard. The relative concentrations, stated as arbitrary unit (AU), were determined via detection by Alkaline Phosphatase (AP)-labeled anti-IgM/anti-IgG (SouthernBiotech), respectively. The p-nitrophenylphosphate (pNPP; Genaxxon) in Diethanolamine buffer was added and data were acquired at 405 nm using a Multiskan FC ELISA plate reader (Thermo Scientific). All samples were measured in duplicates.

For analysis of affinity-maturation, results from plates coated with either peptide(1) or peptide(4) were calculated by dividing peptide(1) by peptide(4). Thus, results were stated as relative units [RU] within the figures.

### Enzyme-Linked Immuno-Spot Assay (ELISpot)

Total splenocytes were measured in triplicates with 300,000 cells/well. ELISpot plates were pre-coated with either native Insulin (Sigma-Aldrich, Cat. 91077C), C-peptide (RoyoBiotech). After 12-24 h incubation of the cells at 37° C., antigen-specific IgM or IgG was detected via anti-IgM-bio:SAV-AP or anti-IgG-bio:SAV-AP (Mabtech). Handling of the plates and antibody concentrations was done according to the manufacturer's recommendations.

### HEp-2 Slides and Fluorescence Microscopy

HEp-2 slides (EUROIMMUN, F191108VA) were used to asses reactivity of serum IgM to nuclear antigens (ANA). Sera of Insulin-A-peptide immunized mice on days 7 and 85 post immunization were diluted to an equal concentration of IgM (approx. 300 ng/mL anti-Insulin-IgM in both immunized samples) and applied onto the HEp-2 slides. Anti-IgM-FITC (eBioscience, Cat. 11-5790-81) was used for detection of ANA-IgM. Stained HEp-2 slides were analyzed using fluorescence microscope Axioskop 2 (Zeiss) and DMi8 software (Leica).

### Glucose Level Monitoring

Assessment of urine glucose levels was done using Combur 10 M Test stripes (Roche Diagnostics, Mannheim). Sterile stripes were used during daily mouse handling and the displayed color after testing was compared to the manufacturer's standard of glucose levels in mmol/L. AccuCheck (Roche Diagnostics, Mannheim) blood glucose monitor was used to measure blood glucose levels of mice. Blood was taken from the tail vein from ad libitum fed mice and transferred onto sterile test stripes. Glucose levels were measured in mmol/L at days stated in the figures for each mouse per group. Control-immunizations were done with littermates and measured at similar times of the day.

### SDS Page, Coomassie and Western Blot

Organs were taken immediately after sacrifice and lysed in RIPA buffer containing protease and phosphatase inhibitors (50 mM TrisHCl, pH 7.4, 1% NP-40, 0.25% sodium deoxycholate, 150 mM NaCl, 1 mM EDTA (pH 8), 1 mM sodium orthovanadate, 1 mM NaF, protease inhibitor cocktail (Sigma-Aldrich). Samples were separated on 10-20% SDS-polyacrylamide gels and either blotted onto PVDF membranes (Millipore) or incubated with Coomassie (Coomassie brilliant blue R-250, ThermoFisher) for 45 min and subsequently de-stained. Subsequently, membranes were blocked for one hour at room temperature in 5% BSA PBS with constant agitation. Primary antibodies were diluted in 5% BSA PBS (BIOMOL Research Laboratories). Secondary antibodies were diluted in 5% BSA PBS. Development of the membrane and recording of the data were done with an optical system Fusion SL (Vilber).

### Pulldown of Total Serum Immunoglobulins

Sera from immunized mice were taken immediately after euthanasia and either IgM or IgG were purified. Removal of antigen bound to antibodies was achieved by repeated freeze-thaw cycles of the serum and pH-shift during elution52. For IgG protein G sepharose beads (Thermo Fisher) were used according to the manufacturers protocol and dialyzed overnight in 10 times sample volume in ix PBS. For IgM, HiTrap IgM columns (GE Healthcare, Sigma-Aldrich) were used according to the manufacturers protocol and dialyzed overnight in 10 times sample volume 1×PBS. Quality check of the isolated immunoglobulins were addressed via SDS page and Coomassie and the amount of insulin-specific immunoglobulins determined via ELISA. Finally, 20-50 μg (1-10 μg insulin-specific-Ig) were injected intravenously.

### Isolation of Insulin-Specific Serum Immunoglobulins

Sera from InsA and control immunized mice were taken immediately after euthanasia and prepared for insulin-specific immunoglobulin isolation. Streptavidin bead columns (Thermo-Scientific, Cat. 21115) were loaded with 10 μg bio-Insulin (BioEagle). The sera were incubated for 90 min at room temperature to ensure binding of insulin-specific antibodies to the beads. Isolation of the insulin-antibodies was done by pH-shift using the manufacturers elution and neutralization solutions. Quality of the isolated immunoglobulins was examined via Coomassie and western blot analysis using anti-IgM heavy chain (Thermo-Scientific, Cat. 62-6820) and anti-IgG heavy chain (Cell Signaling Technologies, Cat. 7076) antibodies. For further in vivo experiments, the isolated antibodies were dialyzed.

### Bio-Layer-Interferometry (BLI)

Interferometric assays (BLItz device, ForteBio) were used to determine the affinity of protein-protein interactions [61]. Here, we used insulin-specific IgM (see isolation of insulin-specific immunoglobulins) and insulin-bio (ThermoFisher) as target. Targets were loaded onto Streptavidin biosensors (ForteBio). Binding affinities of IgM to Insulin were acquired in nm. Subsequently, the calculated affinity value (Ka) was used to determine the dissociation constant (K_{d}): K_{d}=1/Kₐ. Following protocol was used: 30 sec baseline, 30 sec loading, 30 sec baseline, 240 sec association, 120 sec dissociation. For buffering of samples, targets and probes, the manufacturer's sample buffer (ForteBio) was used.

### Wire Hanging Test

The linear wire hanging test is used to assess motor strength and function of mice. Individual mice were put onto a 36 cm elevated horizontal wire above a cage, subsequently the mice tried to stay on the wire by using their paws and muscle strength. The ability in time (sec) of each mouse to stay on the wire was recorded. A maximum time duration of 240 sec was set. Each mouse went through the test three times in a row. The mean value was calculated from the measured data. Blood glucose values were determined before and after the test.

### Statistical Analysis

Graphs were created and statistically analysis was performed by using GraphPad Prism (version 6.0h) software. The numbers of individual replicates or mice (n) are stated within the figure or figure legends. P values were calculated by tests stated in the respective figure legends. Students t-tests with Welch's correction were used to compare two groups within one experiment. P values >0.05 were considered to be statistically significant (n.s.=not significant; *p<0.05; **p<0.01; ***p<0.001, ****p<0.0001).

### REFERENCES

The references are:
1. Goodnow, C. C. et al. Altered immunoglobulin expression and functional silencing of self-reactive B lymphocytes in transgenic mice. Nature 334, 676-682 (1988).
2. Nemazee, D. & Buerki, K. Clonal deletion of autoreactive B lymphocytes in bone marrow chimeras. Proc. Natl. Acad. Sci. U.S.A. 86, 8039-8043 (1989).
3. Lahtela, J. T., Knip, M., Paul, R., Antonen, J. & Salmi, J. Severe antibody-mediated human insulin resistance: Successful treatment with the insulin analog lispro: A case report. Diabetes Care 20, 71-73 (1997).
4. Benschop, R. J., Brandl, E., Chan, A. C. & Cambier, J. C. Unique Signaling Properties of B Cell Antigen Receptor in Mature and Immature B Cells: Implications for Tolerance and Activation. J. Immunol. 167, 4172-4179 (2001).
5. Hartley, S. B. et al. Elimination from peripheral lymphoid tissues of self-reactive B lymphocytes recognizing membrane-bound antigens. Nature 353, 765-769 (1991).
6. Nossal, G. J. V. & Pike, B. L. Mechanisms of clonal abortion tolerogenesis: I. Response of immature hapten-specific B lymphocytes*. J. Exp. Med. 148, 1161-1170 (1978).
7. Wardemann, H. et al. Predominant autoantibody production by early human B cell precursors. Science (80-.). 301, 1374-1377 (2003).
8. Gay, D., Saunders, T., Camper, S. & Weigert, M. Receptor editing: An approach by autoreactive B cells to escape tolerance. J. Exp. Med. 177, 999-1008 (1993).
9. Tiegs, S. L., Russell, D. M. & Nemazee, D. Receptor editing in self-reactive bone marrow B cells. J. Exp. Med. 177, 1009-1020 (1993).
10. Zhang, Z. et al. Contribution of VH gene replacement to the primary B cell repertoire. Immunity 19, 21-31 (2003).
11. Nossal, G. J. V. & Pike, B. L. Clonal anergy: Persistence in tolerant mice of antigen-binding B lymphocytes incapable of responding to antigen or mitogen. Proc. Natl. Acad. Sci. U.S.A. 77, 1602-1606 (1980).
12. Brink, R. et al. Immunoglobulin M and D Antigen Receptors are Both Capable of Mediating B Lymphocyte Activation, Deletion, or Anergy After Interaction with Specific Antigen. J. Exp. Med. 176, 991-1005 (1992).
13. O'Neill, S. K. et al. Monophosphorylation of CD79a and CD79b ITAM motifs initiates a SHIP-1 phosphatase-mediated inhibitory signaling cascade required for B cell anergy. Immunity 35, 746-756 (2011).
14. Lobo, P. I. Role of natural autoantibodies and natural IgM anti-leucocyte autoantibodies in health and disease. Frontiers in Immunology vol. 7 (2016).
15. Grönwall, C., Vas, J. & Silverman, G. J. Protective roles of natural IgM antibodies. Frontiers in Immunology vol. 3 (2012).
16. Hannum, L. G., Ni, D., Haberman, A. M., Weigert, M. G. & Shlomchik, M. J. A disease-related rheumatoid factor autoantibody is not tolerized in a normal mouse: Implications for the origins of autoantibodies in autoimmune disease. J. Exp. Med. 184, 1269-1278 (1996).
17. Wan, X. et al. Pancreatic islets communicate with lymphoid tissues via exocytosis of insulin peptides. Nature 560, 107-111 (2018).
18. Goodnow, C. C., Crosbie, J., Jorgensen, H., Brink, R. A. & Basten, A. Induction of self-tolerance in mature peripheral B lymphocytes. Nature 342, 385-391 (1989).
19. Nemazee, D. A. & Burki, K. Clonal deletion of B lymphocytes in a transgenic mouse bearing anti-MHC class I antibody genes. Nature 337, 562-566 (1989).
20. Shlomchik, M. J. Sites and Stages of Autoreactive B Cell Activation and Regulation. Immunity vol. 28 18-28 (2008).
21. Cooper, G. S. & Stroehla, B. C. The epidemiology of autoimmune diseases. Autoimmunity Reviews vol. 2 119-125 (2003).
22. Suurmond, J. & Diamond, B. Autoantibodies in systemic autoimmune diseases: Specificity and pathogenicity. J. Clin. Invest. 125, 2194-2202 (2015).
23. Übelhart, R. et al. Responsiveness of B cells is regulated by the hinge region of IgD. Nat. Immunol. 16, 534-543 (2015).
24. Setz, C. S. et al. Pten controls B-cell responsiveness and germinal center reaction by regulating the expression of IgD BCR. EMBO J. 38, (2019).
25. Nitschke, L., Kosco, M. H., Kohler, G. & Lamers, M. C. Immunoglobulin D-deficient mice can mount normal immune responses to thymus-independent and -dependent antigens. Proc. Natl. Acad. Sci. U.S.A. 90, 1887-1891 (1993).
26. Kim, Y. M. et al. Monovalent ligation of the B cell receptor induces receptor activation but fails to promote antigen presentation. Proc. Natl. Acad. Sci. U.S.A. 103, 3327-3332 (2006).
27. Yang, J. & Reth, M. Oligomeric organization of the B-cell antigen receptor on resting cells. Nature 467, 465-469 (2010).
28. Maity, P. C. et al. B cell antigen receptors of the IgM and IgD classes are clustered in different protein islands that are altered during B cell activation. Sci. Signal. 8, (2015).
29. Gasparrini, F. et al. Nanoscale organization and dynamics of the siglec CD 22 cooperate with the cytoskeleton in restraining BCR signalling. EMBO J. 35, 258-280 (2016).
30. Zhu, S. et al. Monitoring C-Peptide Storage and Secretion in Islet b-Cells In Vitro and In Vivo. Diabetes 65, 699-709 (2016).
31. Yang, Z. wen, Meng, X. xiao & Xu, P. Central role of neutrophil in the pathogenesis of severe acute pancreatitis. J. Cell. Mol. Med. 19, 2513-2520 (2015).
32. Padoa, C. J. et al. Epitope analysis of insulin autoantibodies using recombinant Fab. Clin. Exp. Immunol. 140, 564-571 (2005).
33. Acharya, P., Lusvarghi, S., Bewley, C. A. & Kwong, P. D. HIV-1 gp120 as a therapeutic target: Navigating a moving labyrinth. Expert Opinion on Therapeutic Targets vol. 19 765-783 (2015).
34. Roes, J. & Rajewsky, K. Immunoglobulin D (IgD)-deficient mice reveal an auxiliary receptor function for IgD in antigen-mediated recruitment of B cells. J. Exp. Med. 177, 45-55 (1993).
35. Gutzeit, C., Chen, K. & Cerutti, A. The enigmatic function of IgD: some answers at last. European Journal of Immunology vol. 48 1101-1113 (2018).
36. Thi Cuc, B., Pohar, J. & Fillatreau, S. Understanding regulatory B cells in autoimmune diseases: the case of multiple sclerosis. Current Opinion in Immunology vol. 61 26-32 (2019).
37. Tokarz, V. L., MacDonald, P. E. & Klip, A. The cell biology of systemic insulin function. Journal of Cell Biology vol. 2171-17 (2018).
38. Pavithran, P. V. et al. Autoantibodies to insulin and dysglycemia in people with and without diabetes: An underdiagnosed association. Clinical Diabetes vol. 34 164-167 (2016).
39. Hu, X. & Chen, F. Exogenous insulin antibody syndrome (EIAS): A clinical syndrome associated with insulin antibodies induced by exogenous insulin in diabetic patients. Endocr. Connect. 7, R47-R55 (2018).
40. Uchigata, Y., Hirata, Y. & Iwamoto, Y. Insulin autoimmune syndrome (Hirata disease): Epidemiology in Asia, including Japan. Diabetol. Int. 1, 21-25 (2010).
41. Uchigata, Y., Eguchi, Y., Takayama-Hasumi, S. & Omori, Y. Insulin autoimmune syndrome (Hirata Disease): clinical features and epidemiology in Japan. Diabetes Research and Clinical Practice vol. 22 89-94 (1994).
42. BERSON, S. A., YALOW, R. S., BAUMAN, A., ROTHSCHILD, M. A. & NEWERLY, K. Insulin-I131 metabolism in human subjects: demonstration of insulin binding globulin in the circulation of insulin treated subjects. J. Clin. Invest. 35, 170-190 (1956).
43. Fineberg, S. E., Kawabata, T., Finco-Kent, D., Liu, C. & Krasner, A. Antibody response to inhaled insulin in patients with type 1 or type 2 diabetes. An analysis of initial phase II and III inhaled insulin (Exubera) trials and a two-year extension trial. J. Clin. Endocrinol. Metab. 90, 3287-3294 (2005).
44. Koyama, R. et al. Hypoglycemia and hyperglycemia due to insulin antibodies against therapeutic human insulin: Treatment with double filtration plasmapheresis and prednisolone. Am. J. Med. Sci. 329, 259-264 (2005).
45. Ganz, M. A. et al. Resistance and allergy to recombinant human insulin. J. Allergy Clin. Immunol. 86, 45-51 (1990).
46. Asai, M. et al. Insulin lispro reduces insulin antibodies in a patient with type 2 diabetes with immunological insulin resistance. Diabetes Res. Clin. Pract. 61, 89-92 (2003).
47. Gupta, S. & Gupta, A. Selective IgM deficiency—An underestimated primary immunodeficiency. Frontiers in Immunology vol. 8 1056 (2017).
48. Köhler, F. et al. Autoreactive B Cell Receptors Mimic Autonomous Pre-B Cell Receptor Signaling and Induce Proliferation of Early B Cells. Immunity 29, 912-921 (2008).
49. Herzog, S. & Jumaa, H. Self-recognition and clonal selection: Autoreactivity drives the generation of B cells. Current Opinion in Immunology vol. 24 166-172 (2012).
50. Wintersteiner. Isoelectric Point of Insulin. 473-478 (1933).
51. Amir, S. et al. Peptide mimotopes of malondialdehyde epitopes for clinical applications in cardiovascular disease. J. Lipid Res. 53, 1316-1326 (2012).
52. Tsiantoulas, D., Gruber, S. & Binder, C. J. B-1 cell immunoglobulin directed against oxidation-specific epitopes. Front. Immunol. 3, 1-6 (2012).
53. Benedict, C. L. & Kearney, J. F. Increased junctional diversity in fetal B cells results in a loss of protective anti-phosphorylcholine antibodies in adult mice. Immunity 10, 607-617 (1999).
54. Nakamura M, Burastero S E, Ueki Y, Larrick J W, Notkins A L, C. P. Probing the Normal and Autoimmune B Cell Repertoire With Epstein-Barr Virus. Frequency of B Cells Producing Monoreactive High Affinity Autoantibodies in Patients With Hashimoto's Disease and Systemic Lupus Erythematosus—PubMed. Journal of Immunology https://pubmed.ncbi.nlm.nih.gov/2848890/(1988).
55. Kaneko, Y., Nimmerjahn, F. & Ravetch, J. V. Anti-inflammatory activity of immunoglobulin G resulting from Fc sialylation. Science (80-.). 313, 670-673 (2006).
56. Reverberi, R. & Reverberi, L. Factors affecting the antigen-antibody reaction. Blood Transfus. 5, 227-240 (2007).
57. Hoppu, S., Ronkainen, M. S., Kimpimäki, T., Simell, S., Korhonen, S., Ilonen, J., . . . & Knip, M. (2004). Insulin autoantibody isotypes during the prediabetic process in young children with increased genetic risk of type 1 diabetes. Pediatric research, 55(2), 236-242.
58. Lupsa, Beatrice C., et al. “Autoimmune forms of hypoglycemia.” Medicine 88.3 (2009): 141-153.
59. Koyama, R., Kato, M., Yamashita, S., Nakanishi, K., Kuwahara, H., & Katori, H. (2005). Hypoglycemia and hyperglycemia due to insulin antibodies against therapeutic human insulin: treatment with double filtration plasmapheresis and prednisolone. The American journal of the medical sciences, 329(5), 259-264.
60. Ikematsu, H., Ichiyoshi, Y., Schettino, E. W., Nakamura, M. & Casali, P. V(H) and VK segment structure of anti-insulin IgG autoantibodies in patients with insulin-dependent diabetes mellitus: Evidence for somatic selection. J. Immunol. 152, 1430-1441 (1994).
61. Kumaraswamy, S. & Tobias, R. Label-free kinetic analysis of an antibody-antigen interaction using biolayer interferometry. in Protein-Protein Interactions: Methods and Applications: Second Edition vol. 1278 165-182 (Springer New York, 2015).

1. An oligomeric anti-insulin antibody, wherein the antibody
(i) has an affinity to insulin and/or proinsulin of Kd<5×10⁻⁷; and/or
(ii) is monospecific for insulin and/or proinsulin.

2. The oligomeric anti-insulin antibody of claim 1, wherein the oligomeric anti-insulin antibody is an anti-insulin antibody of IgM isotype.

3. The oligomeric anti-insulin antibody of claim 1, wherein the oligomeric anti-insulin antibody is chimeric, humanized or human.

4. The oligomeric anti-insulin antibody of claim 1, wherein the antibody comprises
a) a variable heavy (VH) chain comprising CDR1 as defined in SEQ ID NO: 2, CDR2 as defined in SEQ ID NO: 3 and CDR3 as defined in SEQ ID NO: 4 and a variable light (VL) chain comprising CDR1 as defined in SEQ ID NO: 6, CDR2 as defined by the sequence DAS and CDR3 as defined in SEQ ID NO: 7;
b) a variable heavy (VH) chain comprising CDR1 as defined in SEQ ID NO: 9, CDR2 as defined in SEQ ID NO: 10 and CDR3 as defined in SEQ ID NO: 11 and a variable light (VL) chain comprising CDR1 as defined in SEQ ID NO: 13, CDR2 as defined by the sequence GAS and CDR3 as defined in SEQ ID NO: 14; or
c) a variable heavy (VH) chain comprising CDR1 as defined in SEQ ID NO: 16, CDR2 as defined in SEQ ID NO: 17 and CDR3 as defined in SEQ ID NO: 18 and a variable light (VL) chain comprising CDR1 as defined in SEQ ID NO: 20, CDR2 as defined by the sequence DAS and CDR3 as defined in SEQ ID NO: 21.

5. The oligomeric anti-insulin antibody of claim 4, wherein the oligomeric anti-insulin antibody comprises
a) comprises a variable heavy (VH) chain sequence comprising the amino acid sequence of SEQ ID NO: 1 or a sequence having at least 90%, or at least 95% sequence identity to SEQ ID NO: 1 and a variable light (VL) chain sequence comprising the amino acid sequence of SEQ ID NO: 4 or a sequence having at least 90%, preferably or at least 95% sequence identity to SEQ ID NO: 4;
b) comprises a variable heavy (VH) chain sequence comprising the amino acid sequence of SEQ ID NO: 8 or a sequence having at least 90%, or at least 95% sequence identity to SEQ ID NO: 8 and a variable light (VL) chain sequence comprising the amino acid sequence of SEQ ID NO: 12 or a sequence having at least 90%, or at least 95% sequence identity to SEQ ID NO: 12; or
c) comprises a variable heavy (VH) chain sequence comprising the amino acid sequence of SEQ ID NO: 15 or a sequence having at least 90%, or at least 95% sequence identity to SEQ ID NO: 15 and a variable light (VL) chain sequence comprising the amino acid sequence of SEQ ID NO: 19 or a sequence having at least 90%, or at least 95% sequence identity to SEQ ID NO: 19.

6. A polynucleotide that encodes an oligomeric anti-insulin antibody of claim 1.

7. A host cell comprising the polynucleotide of claim 6.

8. A method for producing an oligomeric anti-insulin antibody comprising culturing the host cell of claim 7.

9. A pharmaceutical composition comprising:
(1) the oligomeric anti-insulin antibody of claim 1, a polynucleotide encoding said oligomeric anti-insulin antibody, and/or a host cell comprising the polynucleotide encoding said oligomeric anti-insulin antibody; and
(2) a pharmaceutically acceptable carrier.

10. The pharmaceutical composition of claim 9 comprising a further therapeutic agent.

11. (canceled)

12. (canceled)

13. A method of predicting an insulin-associated disease or disorder, the method comprising the steps of:
(i) determining the affinity of the binding of anti-insulin IgM antibodies to proinsulin and/or insulin from a sample,
wherein the sample has been obtained from a subject, wherein the subject is diagnosed with an insulin-associated disease or disorder or is at risk thereof;
(ii) comparing the level(s) determined in step (i) to a reference value; and
(iii) diagnosing and/or predicting an insulin-associated disease or disorder in said subject based on the comparison made in step (ii), wherein a lower affinity of the binding of anti-insulin IgM antibodies to proinsulin and/or insulin indicates a higher risk for an insulin-associated disease or disorder.

14. A method for determining whether a subject is susceptible to a treatment of insulin-associated disease or disorder, the method comprising the steps of:
(i) determining the affinity of the binding of anti-insulin IgM antibodies to proinsulin and/or insulin from a sample, wherein the sample has been obtained from a subject, wherein the subject is diagnosed with an insulin-associated disease or disorder or is at risk thereof;
(ii) comparing the level(s) determined in step (i) to a reference value; and
(iii) determining whether said subject is susceptible to the treatment of insulin-associated disease or disorder, wherein a lower affinity of the binding of anti-insulin IgM antibodies to proinsulin and/or insulin indicates a higher susceptibility to the treatment of insulin-associated disease or disorder.

15. (canceled)

16. (canceled)

17. A method for producing an oligomeric anti-insulin and/or anti-proinsulin antibody, preferably of the IgM isotype, comprising immunizing a mammal with a mixture of at least one monovalent insulin particle and at least one polyvalent insulin particle.

18. A method for treatment and/or prevention of a disease or disorder, the method comprising a step of administering a therapeutically effective amount of the oligomeric anti-insulin antibody of claim 1.

19. The method of claim 18, wherein the disease or disorder is an insulin-associated disease or disorder.

20. The method of claim 19, wherein the insulin-associated disease or disorder is selected from the group of pancreatic damage, type 1 diabetes, type 2 diabetes, exogenous insulin antibody syndrome, gestational diabetes, and dysglycemia.

21. The method of claim 20, wherein the dysglycemia is dysglycemia in a patient with an insulin-associated disease or disorder selected from the group of pancreatic damage, type 1 diabetes, type 2 diabetes, exogenous insulin antibody syndrome and gestational diabetes.

22. A method for treatment and/or prevention of an insulin-associated disease or disorder, the method comprising a step of administering a therapeutically effective amount of the polynucleotide of claim 6.

23. The method of claim 22, wherein the insulin-associated disease or disorder is selected from the group of pancreatic damage, type 1 diabetes, type 2 diabetes, exogenous insulin antibody syndrome, gestational diabetes, and dysglycemia.

24. The method of claim 23, wherein the dysglycemia is dysglycemia in a patient with an insulin-associated disease or disorder selected from the group of pancreatic damage, type 1 diabetes, type 2 diabetes, exogenous insulin antibody syndrome and gestational diabetes.
